SEQUENCE LISTING (116 sequences)

SEQ ID NO: 1 - DNA, 404 bp, Mus musculus
  Description of Sequenceclone No.1110005I17
gagagcagcccaaaccggacaccatgaggatcccaattcttcccgtcgtggctctcctctctcttctggcattgcatgcggtccagggagcagccctggggcatcccacgatatacccggaagatagcagctacaataattaccctaccgcaacagagggccttaacaatgagttcctgaactttaagaggctacagtctgcctttcagtcagaaaacttcctgaactggcacgtcatcactgatatgttcaaaaatgcatttcctttcattaactgggacttcttccctaagacctaggacctaggctgagcccagtagaagaggaggcaagcatggaatctgaagtccatcctacgacaaatcttccttgcctcagttcccccaataaagtgttttccaccc

SEQ ID NO: 2 - DNA, 613 bp, Mus musculus
  Description of Sequenceclone No.1700007F22
gagcgggtggctacttgaccactgctctgttcgctgcacaggagagctctcccagcagagcaggaagaagagctttagtgatgctgagactggtgctgttgctcctggtcacagactttgcagcctctcatgagactctcgactcttccgattctcaaatcatgaagaggtcacagttccgaacaccagactgtggtcattttgacttcccagcatgccctaggaacctcaaccctgtgtgcggaacggatatgaacacttacagcaatgaatgtaccctgtgcatgaaaatcagggaggacggtagccatattaatatcatcaaagacgagccatgctgatgggcagcttgcagaagagcagacggagaactcctctcctccggtgctccctgctcgaatcccaccccattttcttttccccccatttctttgcatgatacttgttcacagaacttctctgggtagaggctggaagacagtgatgtaaagcggtttataaacttaaaactcttgttctgggcttccggcctacaattcagtttagttcaacttatgacccaggcgacttttgcatcgctttatttcattggaataaaatgagcattgaattc

SEQ ID NO: 3 - DNA, 422 bp, Mus musculus
  Description of Sequenceclone No.1700011J22
gggctgctcctggagatctgagaggtttccgccaggaagtctcggcctccccagtacgccacgaacgttgctttggactcgaagcttcgaagcttcgccttgggccttctcccacgatgaagctcctgctgctgactttggctgcgctcctgctcgtgtcccagctcactccaggtgatgctcagaaatgctggaatctccacggcaagtgccgccaccgatgctccaggaaggagagcgtctacgtctactgcacaaacgggaagatgtgttgcgtgaagcccaaataccagccgaagccaaagccgtggatgttctaactgcccagaagcctgaagcccagataatacagatggcaaagctgtcctaggttgaccccatggattcttgagttcagtcaataaacgtgcttgcccaacccc

SEQ ID NO: 4 - DNA, 372 bp, Mus musculus
  Description of Sequenceclone No.1700056N09
gagatcctgtatcttaagtgacctaacaggtgtgaaagatgttcgacttgaggacgaaggttatgatcggcatcgctagcactctgctgattgctgcaatcatgctgataacgcttgtgttctgtctttaccagaaaatatccaaggccctgaaactcgcaaaggagcctgaatgctgtattgatccatgcaaggaccccaatgagaagatcatccgggccaagcccattattgctgagacttgtcgtaacctcccgtgttgtgacgactgtagcatctataaggatgttggctccctgccaccttgctattgtgtcacaaatgagggactctgacatggcaatggtggacacaaaaatcttcacaaacagt

SEQ ID NO: 5 - DNA, 750 bp, Mus musculus
  Description of Sequenceclone No.2310014H11
gagcaacttatctgcaccaatacccaagacaacaggaatggctgtctcagttcttcgcctgatggttgtcctgggactgagtgccctaatcctgacatgccgggcagatgacaacccaaatgaaaatgacaacccagacagcaagcccgatgactctagtaaaaatccagagccaggtttccccaaattcttaagcatcctgggttcagagatcattgaaaatgcagtggacttcatccttcgctccatgtccagaggatcaagttttatggaacttgaaggcgatcctggacagcaaccttcaaaagtgacctcctaagggcacacccgtttggtaaccatgcagagcaaggacagacagatcctgaacatccagttctgtttcacaagttgtgcagatccaaagacctcaaggcagctcccagcaagtcactgggaattgagtcccagtttcagccaactagaattcattcgagctctttgactttgcattcctctttctgcaaagtcagaaaataagtatactcttagagttttagaattacacacctgacttcatagagatttttgttgttcctaggaattttccatgctgtatttcaatttctaatgagggaaaaatgggattatatatggcatatgcatatgtgcaaaggctaggtcaaggcttcttcatgtatacaatacatacatattaacagactctaaaagataaaataacatatttaaaataaaaagaaaatgaaatgg

SEQ ID NO: 6 - DNA, 513 bp, Mus musculus
  Description of Sequenceclone No.2310031C01
aggggtgagcggagccctgcactggggctcgtagtctccctcagcctctctcagccatgggcgccgtctggtcagccctgctggtcggcgggggtctagctggagcgctcatcctgtggctgctgcggggagactctggggccccggggaaagacggggttgcggagccgccgcagaagggcgcacctcctggggaggctgcggccccgggagacggtccgggtggtggtggcagtggcggcctgagccctgaaccttccgatcgggagctggtctccaaagcaggagccaagtgacttaactttggatacctgccaacatggtctggactgtgggagactttgcaacttgccacttcattctctctcatggtgtgtgctgagactccccaagtgtgcttttgatcgttggctctacatgtgtgctcttagagcagtgttgggaatgttcaaataggtacttgctttttttaaaacagagccttgaactgtacatagaaataaagacaagagc

SEQ ID NO: 7 - DNA, 385 bp, Mus musculus
  Description of Sequenceclone No.4930563B01
gaaggaactctgctgccgtcccctgcaggccacttaccacctccaccctgggtggtgccaccatgaggctggctctcctgcttcttgccatacttgtggcaacggaactggtcgtgtctggcaaaaatcctatccttcaatgcatgggtaacagaggattctgtaggtcttcctgcaaaaagagtgaacaggcctacttctactgcagaactttccagatgtgctgcctccagtcctacgtgaggatcagcctcacaggagtagatgacaacactaactggtcttacgagaagcactggccaagaataccgtgacagcctgcacaccgtgtacagacttccagggaagccgtccctgctgtctgcccccttattaaaatgtatgc

SEQ ID NO: 8 - DNA, 629 bp, Mus musculus
  Description of Sequenceclone No.9130004I05
gctctccctgactcctttcaggcatccaggatcatggcctataaattgcttcaagcagctgtgtgctccaccttgctaatagaagtattgggagcaccatttttgatggaggacccagcaaaccagtttttacgcctgaaaagacacgtggtacacttgccagatttctgggacccagatcatcacccagatgggacaggaacatcattggctgacgaggtctgggaagcatggacttctttgaaagcaagtgcacgccgcaattttgacacggacacattggcctttgatatatctactgcccagtaaatggacttgagtgttacaaaggaaagaacaaacatggtagaagttagcacagtagtgtggtaggcttggagaaacaggaagatcaaatgtggccttcgctacattatgtattatgcccagctcatggagacattgaaaggagcaatggtgaaaacccttgcagacacctgaatgaaactggaaattctctgtaaatgcccattcttgagaatggaaataaagactaaagtaaaagtatttccccaaagaggggcttttctgagaaataggatgcaaatttcaactaacgtaacatttagtgccattaaaacattctgctc

SEQ ID NO: 9 - DNA, 474 bp, Mus musculus
  Description of Sequenceclone No.9230110A19
tgggcagtgagtggcacaccacaggatcccctgctgtctcgggaggatctgaagacatgaagctgcttcaggttctccttgttttgctgtttgtggcacttgcagatggtgcacagcccaaaagatgttttagcaacgtagaaggctactgtaggaagaaatgcagattagtggagatatctgagatgggatgcctgcatgggaaatactgttgtgttaatgagctggagaacaaaaagcacaagaagcactcagtcgttgaggagacagtcaaactccaagacaagtcaaaagtacaagactatatgatcctgcccacggtcacatactacaccatcagtatctgaatgaaccacttgttcacgaaggccgttgtcccctgcagccccatggaatccagtgggctgcttctgtcctgtctctttccttctgtgaacttgagtctgcacacaataaagttcgacccttttgcct

SEQ ID NO: 10 - DNA, 618 bp, Mus musculus
  Description of Sequenceclone No.9230111O07
gagaggttccactgtcctggaactgaggagagagcgccactagccaagtggttatcagcgcctctggctgagaggtatcagcccagatgcccaccaacagctgaacagacaatgaaaatgtggtacaccgtggaattttatcagctctaaaaataagaaattgtaaaaatttcagcaacagctgggaaggcaaaaagaacatctgtagacactcgaggtcaacgtcatgaagccctcgtggttcccgtgtctggtgttcctctgcatgctgctcctgtctgccctgggagggaggaagaacaagtattatcctggagaactattactcgaggaatgctggggccagcccaaaaccaatgactgcgtcaagaagtgttctagaactttcaaatgtgtatacagaaaccacacatgctgctggacctactgtggtaacatctgtgcagaaaatgggaaattttttgaaagaaaatgaccctgacttcccgctggcccgacctcacaggagctagtgggtgcttaagagacttcagaacagggctgggtggttactttcagaaatgctgtttattgggggtacaaaggcaagcatacttgaaaaataaaacacgaaagact

SEQ ID NO: 11 - DNA, 1494 bp, Mus musculus
  Description of Sequenceclone No.A030004E11
gagttgctggtggacatccatgatgaggcccctcctggtgctgttgctgttggctgttgtgtgtgcttctctggccaatcctggtggcatcctggtcatgaagtcctgcgccccaacctgccccaacagcaccgtgtcttcagatggccgtgccctctctgtctcctgctgtcaggggagccagtgcaaccgcagtgctgcctcaggcctgaccggcagccttggggctatatgggccagcgcctctatcagcctactgtgggcactgcttcgagctgcctagtgagggcctgggtggcctttccgtatcatcggggatctgtctgcaacctcagcagcatcctaccccgacccagcccaggctgttactctctctctcatcataaccccaagacacattcaatcttcccttctctcacagaaaggaagttaactacacctgtcccaaaggctcctgccccagagcctagctctcctgcctcaggaaatcttccaagagggcacaaggacacacctccccaagaccctcacctctatgagaccttcagagtcctgggagtacctagctagctaccttcagtaggaagagccgatgtttctcgcaactccctccaccctcaaccttaggcttggcatttgtactaccctctcctctctctgaaaggatgctatatgttggccgtctgggtccctggacctcttctatgtgtgaagcaaccctggcatatggcatgattgtaagatttgggcactgtctaactgaacatcagcttttaaaagatcccctgacttcccatagtatgtgtatgtgtaggggtaaggcagatcatcttgggctttgtgtcaggaattagaatgggtatccattctccctgggcatggagaaacctgctttgtctccctggcagcaacatctcgccactccttccccaatgctgtcctaggagaagaattcatgctcaggtctgcttggtccgaggtattgggtcttgggcatttctggttcttggtcaacttcatcctcagagacatgcaggtggccatgagacagtgccaagtgaaattcttaatgcactaataagcccaggtgcgggagtggtgcctctgccttggactcaggcctcccatgtctgtagtgagtcgcattggccaaaatagaggaaaaagacttctatcacaccccaacctagaagggttccctgagagctcttctgagatatgatgtccctggtgctgctaactggtgaagtgtttgcatctgtcacagtgtgccctggtccccaaaagtccatacttttgtgttggcagcacaacaaaccctcagctttctgaacagtcaccagtactctgaggccactcaaagccacatctccttggggggagggaaggggctcttcttgaggggcagagccagagcccttcaagtctggagcgttttattccctacccctgctgtctttgcctgtccctgtaataaaacactctgacaaaaac

SEQ ID NO: 12 - DNA, 476 bp, Mus musculus
  Description of Sequenceclone No.A430045L05
ggcagtcagctgagaggagcctggcatcatggagaagctgtttgtcttggtctttgctctcgccctgctggctttctcctcagatgcctccccgattctgacagaaaagcaggcaaaacagctcctgaggtccaggcgacaggacaggcccaacaaacctggattcccggacgagcccatgcgggagtatatgcatcatctcctggccctggagcaccgtgctgaggagcagtttctggagcactggctgaatcctcactgcaagccgcactgtgacaggaacattgtccatcctgtgtgaaggacctggggccagctcgcttgaagctcctgacggccttcctgccagttcgcaagtgtaggcagaacgccagacctgccttagaagagggaagtgcatcacgtggggctgctgagtttcgggtcatctgcgttttcctttctttgctgaactaataaaggccagtgcttagtcg

SEQ ID NO: 13 - DNA, 3261 bp, Mus musculus
  Description of Sequenceclone No.A530065I17
ggcatggtccccgggaggcgtaaagagggtgtgtaagtatcggaagagctgtcgggatgcagctggcaagaggaacagtaggaggccgtggctgcgctctctttccactgctgagcatcctagtcgtccagggtgcgcgtatcgtcctctccttggagataagtgccgatgctcacgtccgaggctatgtgggagagaagatcaagttgaaatgcaccttcaagtcatcttcagatgtcactgacaagctgaccatagactggacataccgccctcccagcagcagccgcacagagtctgtgagtgtgggctttcccgaacgtcccctcgagtgcctggcagggtgatacacgcctgtagtctcagtattggtgagatgggaagattaggaaatcgaggccattcctcaacaacatagcaggtttgaaatcagcatgatctacatttaaaggaagaaagggggtaaagtcacctgctgtgcaagtttggttctatccctgagagccatggaggaagtcaacagttgtcttctgacttccacatacaccacatagcactagtgctcctacacgctcacacccccccaaaacacacacactggcacacactcacacacgtgcatgcacacacacacacttccatacacaatcacacacgtgcgttcacacactcacatacacactcacacacaggggtgtgcacacatgtacctgaaatatagttaaacctagaaaaaaagaaataagagaaacttttagacctttggagtgagcctatatcatgccttatatgatatttgtttaattacacagagccttttattctgacatgtaagacttttgtgcattaccttcaacggaaaaaaaaaagggtcagaaaccaagaggaaatgtggtttaattttgttaccttgttgtttccttctgtgtggtgttacagctttttttctttttagatctgatgtactaaaaagggcatggagatgtgggctggagaaattccccactcacatcacagacatcctttctctgagagctactccgatcctgctgcctcagtgggtgtgtctgtctgagaatccacattgcagatttatcttgacttttcctctgcccatgtaaacgtctgcagtgatttatatagctgcctttgaaggaagtggtggcaactagtactggtgacagatgtcactggtgacagatggccttgtgatattagaagcaatatagtttaatcacacacgtaaaacatatttatagttcaaattccattataggtgtttaattagagattacagtgctgtaaaaaccctttctctttatagttccatcagtcgccttggtttaggatagatgctgtttaagaatgcatttgaattggggctgaagaaatggctcagcaagtaagagcactggctgttcttccagagaacctgggttaaattctcggcacccacacaatggttcacaatatcctgtaactccagttccaaggagatcagacaccccgctctgacctccaagggtctgccaggcactatacacagacatacattgcaggcaaaatacccattcatataaaataaaataatttaaaaaattgttaaaaaagaatgtgtcatagccacgtataaattggatctttctttctttatctctttatctctttatttctttgtatatgcatgccacttgcaaggtgccatagctcactgggccatgtcactgtccctagactggattttagaaagcgttctttattctgttttccatctttggaagaagaattccctttcatcttagtgactgtgctttgagttgtgcactcggggctgttactgagtgcctggtgtggagcagggtgggtaccgtagtgaaggaaacaggtctgcagacaaggttccagttgctttagatttaaaatagttcactttattctgggctgtcaggaagctatggctgaccttatttggctctgcttccttgtccaaaagtagggcagagtgagcaataggagcttcttgggtcagaaaccttatatgcagacggcccttctccgagagcactgctctgagaggctgcaggctcatctgaattagaattaagggcatttaagactcttgtaactcaagggtgcagtggtgtggtttgccaggctttgtaagggatcgagaattgtggctcactaacttagcgtgtagtaatccatgagttacatccacctggaaggtcggtgaggatgttttaagcttattatcttgatgatcaatgagaggccttgttttttagacacaaggatttgctccgtgcctcagtttgtttttggatttagtgtatataatccaggctggggtcacattcacagtattcccccctgcctcagcctcctaactaagtacctgggagttgaggcactcagcaccatgttggtttgttaggcctcctctctcttcccacttggagcacttgtcacacatacattagttcttgctgtcttaccaaatgtacgctccatgagaacagagaccatggctgtgtctcggccactgtgttctgttcagtcccacctggaccatgggatgtgtgctaggtatttgtggactgaattcttgatgaaattaaccactgggtgattcagcatcttctctgaggaatagctctaaaaaattgggcaatttggtgtcctaagcaggagggaaccacgtaaacaaagaaagaggaaagcctcctgtcttgcataatggttgtgttgtttgtcatatgtgcctgtcacaccttcagggtaaatatgtgctttagggaccaaatgtccagtcaccaagtccaaagcagtgtcgtcttaatacagcagatattcactgcacgcttatcaggcctatgacgtgaggcttgtgttaactgtcaccgaaggtaacttgagagaggttggattgtgttacacctgcccagtgatttagaaaaaaaaatctcctttttgttacttgccaactgtgtgcctttgagcaacttgcttcatgttgtttgggtcctgtttctcttcctacatgttcagaagagttatagctgcttgctatgctctggtggtggtgtaagggccaagagagtaaacatgtgaaagccatgataaacagaccttggggtaaccttaggacgctgtctccctacttaacgttaaaagttaagcatgtgagcctgtctaatgcaccctgggaagtttagtgatgccttataccaacagatgtgtggaaaagtgtctctgatt

SEQ ID NO: 14 - DNA, 2422 bp, Mus musculus
  Description of Sequenceclone No.A830010B16
ggggcggcgcccagcatggagtggaagctgaatctgctgctctacctagcgctcttcttctttctgctcttcctcctgttcctcctgctcttcgtggtcatcaagcagctgaagaactcggtggccaatacggccgggaccctgcaacccgggcgcctgtccctgcaccgggagccgtggggtttcaataacgagcaagctgtgtgacccagctggcagggtttcagctccgccgggattcagatctatgtcatcgggacccagactggtacccttgaccggtgacacccaggaccgaaactcctatcacagcctaggagccctgcaaagccggggagtggaggagatcttgcaaaagattcttatgtgatctccgtggctccccaatagaaggaggaagcagggaagaaaacagaatgtgcctgtctgagtccatcttccttctaaagagtttcccggggaccaggactacgtcacgggccactgtcttttaacaatatggatgctgttgccttcaatcaaaacccaagctctctagattaggacaaagaatcctgggtggacaagatccagaagtctatgctctagtacaaagcacagttgtgcagaaagcagtgttcaagatggaactcctggttcccacacagccccaccgatgtggctgagcagagtgacgatgtctccctagttctcaagaaatatttgtcaggactggcttcctctctggtcagctgactcccctgtaccaggattttctaggcatgtagcctggaagccttacatttgtgtgtgttgggggatctggaggttgagggggtcttcataatctcctcacggaactttggtaacagatcaacataggttgttttcttttgctcctttttggtcccctccatcacgttccagttagttcaattttggtggcagctaagtttgtgtatttgatccctataaaggccatcccttccatgcacaaggaaagacactctgctcagaaaggctacctctcttccttccaccctcgatccaggaatatcccgggagattgcctctcgagctgcttctactcacagcctacgtactggccaagccgagggagaagaaacacttcccacacccagagctgtgcaaaagaactttctgtgatatgaaactgttctatatctgtggtgcccaatacggcaactactagtcacatgtgtctagtaaacactgaaatatggctaacggggcttagcttttatttaatttcacccgagacagccacttggggctagtggctattgtatgtacagttcacaccattctcgtgtgtgtgtgtgtgtgtgtgtgtgtgtgtgtgtgtgtgtattagagtcaaagaggggaggtggctgctagccatgaactccacatcccatggtcttcattgtttttcttgctttcagtttttctccttgatgaaaaagaaagtcagtatgttagctctaaattgagaatgctaccatctacctattgcatgtcttcttgtaccaaatgagataggtttcagtgataattgaccatggtaaaaatggaagtgttgattcactacatctccatattcccagtccttgcccctgaccaaggtcacattgtcctaggcactttattccattttagttctctttttaatattgtgaatcccaagcaattattgtatatgtttttcaaaatacctaatggtttatattttttaaggctgtaaaataaaaattgacaattacatgtaactcaggttttgtgtgtatgtgtgtctgtgcaatttgtagtaacttactacccaaactttattgtattgattaggatatgaataaatggaaagtgatattgatttttgtatcagatttcaaatttttttttccaagacggggtttctctgtgttctctgtgtagctctgactgtcctggaactcactttgtagaccaggctagcctcgaactcagacatccgcctgcctctgcctcctgagtgctgggattaaaggtgtgatccaccacgcccagctggtggattttcattttaatggacatatttatacgccaatgaatactttatttaaaaaaatattaaatgagtcttctgttatggtcaaagcttgaataacagtagttgtctccaggctcccagtttgtattgagttttagaaattattagaccgccgcagacatattcctttcacagacgcatagtctcccacctgtggttaatctatggatttaattgcaaagattttttaaaaggtgtattattaattttgaagtttttaatttatccatgttttgttcattgctttctgtgtcctctttaggtgacatttgcctactctgccctcaccctggctaccaaacgggattctgctctgccttcttccatactagaagtatagttttaacttttact

SEQ ID NO: 15 - DNA, 625 bp, Mus musculus
  Description of Sequenceclone No.B230114O10
ggcgcgcgtgcactccgcggtacagtctcgtgcgcgcatagtcgtgggaaggaacgaggggctccagcgtctccctgagtggctgagggagcgaggaaccgccgagccctccttcccctgctcggcgggcgcaggctgcagctagcagctctccaccatgcccgggggcgtaccctggtccgcctacttgaaaatgctttcctccagcctcctggccatgtgcgccggggcccaggtggtgcattggtactatcggcctgacctgacaatacctgaaattccaccaaagcctggagaactcaaaacggagcttttgggactgaaagagagacgccacgaacctcatgtttcacagcagtagaaacttcaaaataacagctgtgccaagaattctgtgaataatgtttcaaatatgtattttaaaatttattaagtaaaactactttttaaaacaccgaatttagggggctcaatggctcagaggttaagagcactgtctgctcttccagaggtcctgtgttcaattcgcagcaaccacttggtggttcataaccatctatgagttgtaccctgttctggcccgcaggcagaatgctgtatacataataaatcttaatattttttt

SEQ ID NO: 16 - DNA, 570 bp, Mus musculus
  Description of Sequenceclone No.B230352O20
ggcccagactgccccagtgagctggagcattgaagaagagtctcctgccaataacactgaaaagaaagaaaaaggagcaagagccatgatgaaatctgtggtacttgtcatccttgggctaactttgctgttagaaacacaagccatgccttcaagtcgcctctcctgctacagaaagttgctaaaggatcgcaattgtcacaaccttccggagggcagagccgacctgaagctgatagatgcaaatgtccagcatcatttctgggatgggaagggatgcgagatgatctgctactgcaacttcagcgaactgctctgctgcccaaaagatgtcttctttggaccaaagatctcctttgtgatcccctgcaacaaccactgaggatctgccttgcactctggagaacatggtcctgaaggccttcacgtcccctaatttcccacaaactctgtcagttcagcgccatttctgatatccatccagtatatccaatcttgcatagattctataaagtcttacttgctagagtatacttgggctaaagtggtaataaaagttgtttccatttg

SEQ ID NO: 17 - DNA, 447 bp, Mus musculus
  Description of Sequenceclone No.C230071E12
taatgtggcacaacgtggggctgaccctgctggtgttcgtggccacgctgctgatcgtcctgttgctgatggtgtgcggttggtattttgtatggcatctatttttatctaaattcaagtttcttcgggagcttgtgggagacacaggatcccaggaaggagataatgagcagccttcagggtctgaaacagaagaagacccttcggcttcaccacagaagatcagatctgctcgccagagaaggccacctgttgacgccggccactgagcagacaaagcagtgtcttagagtgtgggccaaggcagtcacgagcctctgtccttagtggcgacctagctttgaaagttactaagtgaccgaggaacatttgcaattggatttatatccagttttaaaaaaaaaagatttacacgtaagccatatagaaataaagggaatttaaacc

SEQ ID NO: 18 - DNA, 615 bp, Mus musculus
  Description of Sequenceclone No.C630041L24
ggtccttctgacgtctttaacactgagctgaggctccttcagtcaggagtgtcttccacaggtgaaagcaagcaaagccagagggcaaataactgcttccttccacaggtctgtcaccatgaaggtgatcttctcagttgcagtccttgttctggccagctcagtgtggacttcccttgcagttgatttcatccttcctatgaactttcacatgaccggggagcttctacaaaagacaaaggccttgtgcatcaagaatatacagttatgttggatacttagctacttcaaggtcagtgagcctatatgtggcagcaaccaagtgacctacgagggcgagtgccatctctgctccggaattctgaacactcctcggatgagtctgaacactgataaaagttactgagttctcagagaggcacgtggagcacgactgactcttcaaagatacccagtttgggagacagggtctggagttcaatggtagtgtgcactagggtgtacgaagccttagggagtgttaaaagagaacagcaccttgagaaaaacatcttgatattagaagatacttggatcaaattcattgatttctttcttcaataaatgattctcagc

SEQ ID NO: 19 - DNA, 509 bp, Mus musculus
  Description of Sequenceclone No.D630020P16
ggagagttttagctttctttcgcatacggaggacagttctgctgggtccttctgagggcggccctcccatgaagctgacataaccacacctggcccccacgctcacctgcacagttttcttgggagatgaagctgcttggcctctctctactcgcagtgaccattctgctttgctgtaacatggctcgacctgaaataaagaagaagaacgttttttccaaacctggctattgcccagagtatcgggttccctgcccctttgtccttatacctaaatgcaggcgtgataaaggctgcaaggacgccctgaagtgttgcttcttctactgccagatgcgctgtgtggatccatgggagagcccagaatagacaaaccggagaaaacacatgtgattcagtttcagtcgaggatagccggcctctgtttcctcctccgagaggcccatcctgaaactaaagattaagtgcttgttaatatgagtacaaaataaaggaataaagtgttgtgt

SEQ ID NO: 20 - protein, 91 aa, Mus musculus
  Description of Sequenceclone No.1110005I17
MRIPILPVVALLSLLALHAVQGAALGHPTIYPEDSSYNNYPTATEGLNNEFLNFKRLQSAFQSENFLNWHVITDMFKNAFPFINWDFFPKT

SEQ ID NO: 21 - protein, 86 aa, Mus musculus
  Description of Sequenceclone No.1700007F22
MLRLVLLLLVTDFAASHETLDSSDSQIMKRSQFRTPDCGHFDFPACPRNLNPVCGTDMNTYSNECTLCMKIREDGSHINIIKDEPC

SEQ ID NO: 22 - protein, 67 aa, Mus musculus
  Description of Sequenceclone No.1700011J22
MKLLLLTLAALLLVSQLTPGDAQKCWNLHGKCRHRCSRKESVYVYCTNGKMCCVKPKYQPKPKPWMF

SEQ ID NO: 23 - protein, 98 aa, Mus musculus
  Description of Sequenceclone No.1700056N09
MFDLRTKVMIGIASTLLIAAIMLITLVFCLYQKISKALKLAKEPECCIDPCKDPNEKIIRAKPIIAETCRNLPCCDDCSIYKDVGSLPPCYCVTNEGL

SEQ ID NO: 24 - protein, 93 aa, Mus musculus
  Description of Sequenceclone No.2310014H11
MAVSVLRLMVVLGLSALILTCRADDNPNENDNPDSKPDDSSKNPEPGFPKFLSILGSEIIENAVDFILRSMSRGSSFMELEGDPGQQPSKVTS

SEQ ID NO: 25 - protein, 79 aa, Mus musculus
  Description of Sequenceclone No.2310031C01
MGAVWSALLVGGGLAGALILWLLRGDSGAPGKDGVAEPPQKGAPPGEAAAPGDGPGGGGSGGLSPEPSDRELVSKAGAK

SEQ ID NO: 26 - protein, 83 aa, Mus musculus
  Description of Sequenceclone No.4930563B01
MRLALLLLAILVATELVVSGKNPILQCMGNRGFCRSSCKKSEQAYFYCRTFQMCCLQSYVRISLTGVDDNTNWSYEKHWPRIP

SEQ ID NO: 27 - protein, 91 aa, Mus musculus
  Description of Sequenceclone No.9130004I05
MAYKLLQAAVCSTLLIEVLGAPFLMEDPANQFLRLKRHVVHLPDFWDPDHHPDGTGTSLADEVWEAWTSLKASARRNFDTDTLAFDISTAQ

SEQ ID NO: 28 - protein, 96 aa, Mus musculus
  Description of Sequenceclone No.9230110A19
MKLLQVLLVLLFVALADGAQPKRCFSNVEGYCRKKCRLVEISEMGCLHGKYCCVNELENKKHKKHSVVEETVKLQDKSKVQDYMILPTVTYYTISI

SEQ ID NO: 29 - protein, 82 aa, Mus musculus
  Description of Sequenceclone No.9230111O07
MKPSWFPCLVFLCMLLLSALGGRKNKYYPGELLLEECWGQPKTNDCVKKCSRTFKCVYRNHTCCWTYCGNICAENGKFFERK

SEQ ID NO: 30 - protein, 87 aa, Mus musculus
  Description of Sequenceclone No.A030004E11
MMRPLLVLLLLAVVCASLANPGGILVMKSCAPTCPNSTVSSDGRALSVSCCQGSQCNRSAASGLTGSLGAIWASASISLLWALLRAA

SEQ ID NO: 31 - protein, 90 aa, Mus musculus
  Description of Sequenceclone No.A430045L05
MEKLFVLVFALALLAFSSDASPILTEKQAKQLLRSRRQDRPNKPGFPDEPMREYMHHLLALEHRAEEQFLEHWLNPHCKPHCDRNIVHPV

SEQ ID NO: 32 - protein, 96 aa, Mus musculus
  Description of Sequenceclone No.A530065I17
MQLARGTVGGRGCALFPLLSILVVQGARIVLSLEISADAHVRGYVGEKIKLKCTFKSSSDVTDKLTIDWTYRPPSSSRTESVSVGFPERPLECLAG

SEQ ID NO: 33 - protein, 63 aa, Mus musculus
  Description of Sequenceclone No.A830010B16
MEWKLNLLLYLALFFFLLFLLFLLLFVVIKQLKNSVANTAGTLQPGRLSLHREPWGFNNEQAV

SEQ ID NO: 34 - protein, 67 aa, Mus musculus
  Description of Sequenceclone No.B230114O10
MPGGVPWSAYLKMLSSSLLAMCAGAQVVHWYYRPDLTIPEIPPKPGELKTELLGLKERRHEPHVSQQ

SEQ ID NO: 35 - protein, 98 aa, Mus musculus
  Description of Sequenceclone No.B230352O20
MMKSVVLVILGLTLLLETQAMPSSRLSCYRKLLKDRNCHNLPEGRADLKLIDANVQHHFWDGKGCEMICYCNFSELLCCPKDVFFGPKISFVIPCNNH

SEQ ID NO: 36 - protein, 88 aa, Mus musculus
  Description of Sequenceclone No.C230071E12
MWHNVGLTLLVFVATLLIVLLLMVCGWYFVWHLFLSKFKFLRELVGDTGSQEGDNEQPSGSETEEDPSASPQKIRSARQRRPPVDAGH

SEQ ID NO: 37 - protein, 95 aa, Mus musculus
  Description of Sequenceclone No.C630041L24
MKVIFSVAVLVLASSVWTSLAVDFILPMNFHMTGELLQKTKALCIKNIQLCWILSYFKVSEPICGSNQVTYEGECHLCSGILNTPRMSLNTDKSY

SEQ ID NO: 38 - protein, 80 aa, Mus musculus
  Description of Sequenceclone No.D630020P16
MKLLGLSLLAVTILLCCNMARPEIKKKNVFSKPGYCPEYRVPCPFVLIPKCRRDKGCKDALKCCFFYCQMRCVDPWESPE

SEQ ID NO: 39 - DNA, 16 bp, Artificial Sequence
  Description of Artificial SequenceSynthetic DNA
gccaattgccgccacc

SEQ ID NO: 40 - DNA, 17 bp, Artificial Sequence
  Description of Artificial SequenceSynthetic DNA
catccttcggtggcggc

SEQ ID NO: 41 - DNA, 18 bp, Artificial Sequence
  Description of Artificial SequenceSynthetic DNA
gccgccaccgaaggaatg

SEQ ID NO: 42 - DNA, 17 bp, Artificial Sequence
  Description of Artificial SequenceSynthetic DNA
catccttcggtggcggc

SEQ ID NO: 43 - DNA, 27 bp, Artificial Sequence
  Description of Artificial SequenceSynthetic DNA
agcggataacaatttcacacaggaaac

SEQ ID NO: 44 - DNA, 20 bp, Artificial Sequence
  Description of Artificial SequenceSynthetic DNA
aactcctggttcccacacag

SEQ ID NO: 45 - DNA, 20 bp, Artificial Sequence
  Description of Artificial SequenceSynthetic DNA
atcccccaacacacacaaat

SEQ ID NO: 46 - DNA, 20 bp, Artificial Sequence
  Description of Artificial SequenceSynthetic DNA
gagacacaggatcccaggaa

SEQ ID NO: 47 - DNA, 20 bp, Artificial Sequence
  Description of Artificial SequenceSynthetic DNA
ttcaaagctaggtcgccact

SEQ ID NO: 48 - DNA, 20 bp, Artificial Sequence
  Description of Artificial SequenceSynthetic DNA
gacactcgaggtcaacgtca

SEQ ID NO: 49 - DNA, 20 bp, Artificial Sequence
  Description of Artificial SequenceSynthetic DNA
cagcagcatgtgtggtttct

SEQ ID NO: 50 - DNA, 20 bp, Artificial Sequence
  Description of Artificial SequenceSynthetic DNA
ttcgacttgaggacgaaggt

SEQ ID NO: 51 - DNA, 20 bp, Artificial Sequence
  Description of Artificial SequenceSynthetic DNA
acacgggaggttacgacaag

SEQ ID NO: 52 - DNA, 20 bp, Artificial Sequence
  Description of Artificial SequenceSynthetic DNA
gctgcttcaggttctccttg

SEQ ID NO: 53 - DNA, 20 bp, Artificial Sequence
  Description of Artificial SequenceSynthetic DNA
cgactgagtgcttcttgtgc

SEQ ID NO: 54 - DNA, 20 bp, Artificial Sequence
  Description of Artificial SequenceSynthetic DNA
gcaaaacagctcctgaggtc

SEQ ID NO: 55 - DNA, 20 bp, Artificial Sequence
  Description of Artificial SequenceSynthetic DNA
aggtccttcacacaggatgg

SEQ ID NO: 56 - DNA, 20 bp, Artificial Sequence
  Description of Artificial SequenceSynthetic DNA
tacttgtggcaacggaactg

SEQ ID NO: 57 - DNA, 20 bp, Artificial Sequence
  Description of Artificial SequenceSynthetic DNA
gcaggctgtcacggtattct

SEQ ID NO: 58 - DNA, 20 bp, Artificial Sequence
  Description of Artificial SequenceSynthetic DNA
ctggtgctgctaactggtga

SEQ ID NO: 59 - DNA, 20 bp, Artificial Sequence
  Description of Artificial SequenceSynthetic DNA
agacagcaggggtagggaat

SEQ ID NO: 60 - DNA, 20 bp, Artificial Sequence
  Description of Artificial SequenceSynthetic DNA
gcggaacggatatgaacact

SEQ ID NO: 61 - DNA, 20 bp, Artificial Sequence
  Description of Artificial SequenceSynthetic DNA
aagaaaatggggtgggattc

SEQ ID NO: 62 - DNA, 20 bp, Artificial Sequence
  Description of Artificial SequenceSynthetic DNA
caagtcgcctctcctgctac

SEQ ID NO: 63 - DNA, 20 bp, Artificial Sequence
  Description of Artificial SequenceSynthetic DNA
ggcagatcctcagtggttgt

SEQ ID NO: 64 - DNA, 20 bp, Artificial Sequence
  Description of Artificial SequenceSynthetic DNA
cctggctattgcccagagta

SEQ ID NO: 65 - DNA, 20 bp, Artificial Sequence
  Description of Artificial SequenceSynthetic DNA
cggctatcctcgactgaaac

SEQ ID NO: 66 - DNA, 20 bp, Artificial Sequence
  Description of Artificial SequenceSynthetic DNA
tgagcctatatgtggcagca

SEQ ID NO: 67 - DNA, 20 bp, Artificial Sequence
  Description of Artificial SequenceSynthetic DNA
cctgtctcccaaactgggta

SEQ ID NO: 68 - DNA, 20 bp, Artificial Sequence
  Description of Artificial SequenceSynthetic DNA
tctccctcagcctctctcag

SEQ ID NO: 69 - DNA, 20 bp, Artificial Sequence
  Description of Artificial SequenceSynthetic DNA
ccacagtccagaccatgttg

SEQ ID NO: 70 - DNA, 20 bp, Artificial Sequence
  Description of Artificial SequenceSynthetic DNA
ttaccctaccgcaacagagg

SEQ ID NO: 71 - DNA, 20 bp, Artificial Sequence
  Description of Artificial SequenceSynthetic DNA
gaggcaaggaagatttgtcg

SEQ ID NO: 72 - DNA, 20 bp, Artificial Sequence
  Description of Artificial SequenceSynthetic DNA
tgcaccaatacccaagacaa

SEQ ID NO: 73 - DNA, 20 bp, Artificial Sequence
  Description of Artificial SequenceSynthetic DNA
tctctgaacccaggatgctt

SEQ ID NO: 74 - DNA, 20 bp, Artificial Sequence
  Description of Artificial SequenceSynthetic DNA
gtcctaagcaggagggaacc

SEQ ID NO: 75 - DNA, 20 bp, Artificial Sequence
  Description of Artificial SequenceSynthetic DNA
cctgataagcgtgcagtgaa

SEQ ID NO: 76 - DNA, 20 bp, Artificial Sequence
  Description of Artificial SequenceSynthetic DNA
tcactccaggtgatgctcag

SEQ ID NO: 77 - DNA, 20 bp, Artificial Sequence
  Description of Artificial SequenceSynthetic DNA
cctaggacagctttgccatc

SEQ ID NO: 78 - DNA, 20 bp, Artificial Sequence
  Description of Artificial SequenceSynthetic DNA
ctgtgtgctccaccttgcta

SEQ ID NO: 79 - DNA, 20 bp, Artificial Sequence
  Description of Artificial SequenceSynthetic DNA
agccaatgatgttcctgtcc

SEQ ID NO: 80 - DNA, 20 bp, Artificial Sequence
  Description of Artificial SequenceSynthetic DNA
ttggtactatcggcctgacc

SEQ ID NO: 81 - DNA, 20 bp, Artificial Sequence
  Description of Artificial SequenceSynthetic DNA
ccccctaaattcggtgtttt

SEQ ID NO: 82 - DNA, 36 bp, Artificial Sequence
  Description of Artificial SequenceSynthetic DNA
cattccttcggtggcggcgtggccggcgtgaacagg

SEQ ID NO: 83 - DNA, 43 bp, Artificial Sequence
  Description of Artificial SequenceSynthetic DNA
cattccttcggtggcggcttttctttcaaaaaatttcccattt

SEQ ID NO: 84 - DNA, 38 bp, Artificial Sequence
  Description of Artificial SequenceSynthetic DNA
cattccttcggtggcggcgagtccctcatttgtgacac

SEQ ID NO: 85 - DNA, 38 bp, Artificial Sequence
  Description of Artificial SequenceSynthetic DNA
cattccttcggtggcggccacaggatggacaatgttcc

SEQ ID NO: 86 - DNA, 38 bp, Artificial Sequence
  Description of Artificial SequenceSynthetic DNA
cattccttcggtggcggcctaggcagctcgaagcagtg

SEQ ID NO: 87 - DNA, 38 bp, Artificial Sequence
  Description of Artificial SequenceSynthetic DNA
cattccttcggtggcggcgctaggtactcccaggactc

SEQ ID NO: 88 - DNA, 37 bp, Artificial Sequence
  Description of Artificial SequenceSynthetic DNA
cattccttcggtggcggcgtggttgttgcaggggatc

SEQ ID NO: 89 - DNA, 39 bp, Artificial Sequence
  Description of Artificial SequenceSynthetic DNA
cattccttcggtggcggcgtaacttttatcagtgttcag

SEQ ID NO: 90 - DNA, 37 bp, Artificial Sequence
  Description of Artificial SequenceSynthetic DNA
cattccttcggtggcggccttggctcctgctttggag

SEQ ID NO: 91 - DNA, 38 bp, Artificial Sequence
  Description of Artificial SequenceSynthetic DNA
cattccttcggtggcggcggtcttagggaagaagtccc

SEQ ID NO: 92 - DNA, 39 bp, Artificial Sequence
  Description of Artificial SequenceSynthetic DNA
cattccttcggtggcggcggaggtcacttttgaaggttg

SEQ ID NO: 93 - DNA, 36 bp, Artificial Sequence
  Description of Artificial SequenceSynthetic DNA
cattccttcggtggcggcccctgccaggcactcgag

SEQ ID NO: 94 - DNA, 37 bp, Artificial Sequence
  Description of Artificial SequenceSynthetic DNA
cattccttcggtggcggcgaacatccacggctttggc

SEQ ID NO: 95 - DNA, 40 bp, Artificial Sequence
  Description of Artificial SequenceSynthetic DNA
cattccttcggtggcggcctgggcagtagatatatcaaag

SEQ ID NO: 96 - DNA, 39 bp, Artificial Sequence
  Description of Artificial SequenceSynthetic DNA
cattccttcggtggcggcctgctgtgaaacatgaggttc

SEQ ID NO: 97 - DNA, 34 bp, Artificial Sequence
  Description of Artificial SequenceSynthetic DNA
gccgccaccgaaggaatggggctcagtgatgggg

SEQ ID NO: 98 - DNA, 35 bp, Artificial Sequence
  Description of Artificial SequenceSynthetic DNA
gccaattgccgccaccatgtggcacaacgtggggc

SEQ ID NO: 99 - DNA, 35 bp, Artificial Sequence
  Description of Artificial SequenceSynthetic DNA
gccaattgccgccaccatgaagccctcgtggttcc

SEQ ID NO: 100 - DNA, 37 bp, Artificial Sequence
  Description of Artificial SequenceSynthetic DNA
gccaattgccgccaccatgttcgacttgaggacgaag

SEQ ID NO: 101 - DNA, 37 bp, Artificial Sequence
  Description of Artificial SequenceSynthetic DNA
gccaattgccgccaccatggagaagctgtttgtcttg

SEQ ID NO: 102 - DNA, 35 bp, Artificial Sequence
  Description of Artificial SequenceSynthetic DNA
gccaattgccgccaccatgatgaggcccctcctgg

SEQ ID NO: 103 - DNA, 35 bp, Artificial Sequence
  Description of Artificial SequenceSynthetic DNA
gccaattgccgccaccatgggccagcgcctctatc

SEQ ID NO: 104 - DNA, 38 bp, Artificial Sequence
  Description of Artificial SequenceSynthetic DNA
gccaattgccgccaccatgatgaaatctgtggtacttg

SEQ ID NO: 105 - DNA, 38 bp, Artificial Sequence
  Description of Artificial SequenceSynthetic DNA
gccaattgccgccaccatgaaggtgatcttctcagttg

SEQ ID NO: 106 - DNA, 35 bp, Artificial Sequence
  Description of Artificial SequenceSynthetic DNA
gccaattgccgccaccatgggcgccgtctggtcag

SEQ ID NO: 107 - DNA, 37 bp, Artificial Sequence
  Description of Artificial SequenceSynthetic DNA
gccaattgccgccaccatgaggatcccaattcttccc

SEQ ID NO: 108 - DNA, 36 bp, Artificial Sequence
  Description of Artificial SequenceSynthetic DNA
gccaattgccgccaccatggctgtctcagttcttcg

SEQ ID NO: 109 - DNA, 36 bp, Artificial Sequence
  Description of Artificial SequenceSynthetic DNA
gccaattgccgccaccatgcagctggcaagaggaac

SEQ ID NO: 110 - DNA, 36 bp, Artificial Sequence
  Description of Artificial SequenceSynthetic DNA
gccaattgccgccaccatgaagctcctgctgctgac

SEQ ID NO: 111 - DNA, 38 bp, Artificial Sequence
  Description of Artificial SequenceSynthetic DNA
gccaattgccgccaccatggcctataaattgcttcaag

SEQ ID NO: 112 - DNA, 33 bp, Artificial Sequence
  Description of Artificial SequenceSynthetic DNA
gccaattgccgccaccatgcccgggggcgtacc

SEQ ID NO: 113 - DNA, 47 bp, Artificial Sequence
  Description of Artificial SequenceSynthetic DNA
gagcgcgcgtaatgcgagtcactatagggccaattgccgccaccatg

SEQ ID NO: 114 - DNA, 30 bp, Artificial Sequence
  Description of Artificial SequenceSynthetic DNA
gagcgcgcgtaatgcgagtcactatagggc

SEQ ID NO: 115 - DNA, 21 bp, unknown
  Sample nucleotide sequence to show percent identity calculations (page 17 of the specification)
gcgcgaaatactcactcgagg

SEQ ID NO: 116 - DNA, 22 bp, unknown
  Sample nucleotide sequence to show percent identity calculations (page 17 of the specification)
tatagccctaccactagagtcc

(19) United States
(12) Patent Application Publication
(10) Pub. No.: US 2004/0053306 A1
(43) Pub. Date: Mar. 18, 2004

(54) Polynucleotides encoding short polypeptides, polypeptides encoded thereby, and methods of use thereof

(76) Inventors: Yoshihide Hayashizaki, Ibaraki (JP); Mamoru Kamiya, Tokyo (JP)

Correspondence Address: BIRCH STEWART KOLASCH & BIRCH, PO BOX 747, FALLS CHURCH, VA, 22040-0747, US

(21) Appl. No.: 10/462,691

(22) Filed: Jun. 17, 2003

Related U.S. Application Data
(60) Provisional application No. 60/389,145, filed on Jun. 17, 2002.

(51) Int. Cl. C12Q 1/68; C07H 21/04; C07K 14/47

(52) U.S. Cl. 435/6; 435/69.1; 435/320.1; 435/325; 530/350; 536/23.2

(57) ABSTRACT

Novel polynucleotides and polypeptides related thereto, as well as nucleic acid compositions encoding the same, are provided. The subject polypeptide and nucleic acid compositions find use in a variety of applications, including diagnostic applications, and therapeutic agent screening applications, as well as in treatment of a variety of disease conditions. Also provided are methods of modulating a biological activity of a subject polypeptide and methods of treating disease conditions associated therewith, particularly by administering modulators of the subject polypeptides.

### FIELD OF THE INVENTION

The present invention relates to novel polynucleotides obtained from a full-length mouse cDNA libraries. The polynucleotides encode short polypeptides which have various functions.

### BACKGROUND ART

Full length cDNA libraries have been developed for many organisms. Full length cDNA has completely information for producing a protein unlike expressed sequence tag. In the full length cDNA libraries many novel genes have been found. However, it has been difficult to explore a gene of which nucleotide length is short and encoding a short protein because of noise data. Thus, even under the circumstance where many cDNA libraries have been developed, many genes of which nucleotide length are short have not been found.

### SUMMARY OF THE INVENTION

The present inventors has concentrated to explore short length genes using full length cDNA libraries and have obtained many short length genes which are novel and various functions.

Thus, the present inventions provide novel polynucleotides and polypeptides related thereto, as well as nucleic acid compositions encoding the same. The subject polypeptide and nucleic acid compositions can be used in a variety of applications, including diagnostic applications, and therapeutic agent screening applications, as well as in treatment of a variety of disease conditions. Also provided are methods of modulating a biological activity of a subject polypeptide and methods of treating disease conditions associated therewith, particularly by administering modulators of the subject polypeptides.

The aspect of the present invention is an isolated polynucleotide that encodes a subject polypeptide. In some embodiments, the polypeptide has at least about 70%, at least about 75%, at least about 80%, at least about 85%, at least about 90%, at least about 95%, or at least about 98% amino acid sequence identity with the amino acid encoded by a polynucleotide sequence shown in the Sequence listing. In some embodiments, the polypeptide has an amino acid sequence encoded by a polynucleotide sequence shown in the Sequence listing. In many embodiments, the polypeptide has at least one activity associated with the naturally occurring encoded polypeptide.

The another aspect of the present invention is an isolated polynucleotide that hybridizes under stringent hybridization conditions to a coding region of a nucleotide sequence shown in the Sequence listing, or a complement thereof.

The another aspect of the present invention is an isolated polynucleotide that shares at least about 70%, at least about 75%, at least about 80%, at least about 85%, at least about 90%, at least about 95%, or at least about 98% nucleotide sequence identity with a nucleotide sequence of the coding region of a sequence shown in the Sequence listing, or a complement thereof. In some embodiments, a subject polynucleotide has the nucleotide sequence shown in the Sequence listing, or a coding region thereof.

The further aspect of the present invention is a recombinant vector that includes a subject polynucleotide.

The further aspect of the present invention is host cells, e.g., isolated in vitro host cells, and in vivo host cells, that comprise a polynucleotide of the invention, or a recombinant vector of the invention. The further aspect of the present invention is a method of producing a polypeptide of the invention, the method involving culturing a subject host cell under conditions such that the subject polypeptide is produced by the host cells; and recovering the subject polypeptide from the culture, e.g., from within the host cells, or from the culture medium. The further aspect of the present invention is a method of producing a polypeptide of the invention, the method involving in vitro cell-free transcription and/or translation.

The further aspect of the present invention is a pair of isolated nucleic acid molecules, each from about 10 to 200 nucleotides in length. The first nucleic acid molecule of the pair comprises a sequence of at least 10 contiguous nucleotides having 100% sequence identity to a nucleic acid sequence shown in the Sequence listing. The second nucleic acid molecule of the pair comprises a sequence of at least 10 contiguous nucleotides having 100% sequence identity to the reverse complement of a nucleic acid sequence shown in the Sequence listing. The sequence of said second nucleic acid molecule is located 3′ of the nucleic acid sequence of the first nucleic acid molecule shown in the Sequence listing. The pair of isolated nucleic acid molecules are useful in a polymerase chain reaction to amplify a nucleic acid that has sequence identity to the sequences shown in the Sequence listing, particularly when cDNA is used as a template.

The further aspect of the present invention is an isolated polypeptide, e.g., an isolated polypeptide encoded by a subject polynucleotide. In some embodiments, the polypeptide is a fusion protein. In some embodiments, the polypeptide has one or more amino acid substitutions and/or insertions and/or deletions, compared with a sequence shown in sequence listing. In some embodiments, the polypeptide has an amino acid sequence shown in sequence listing.

The further aspect of the present invention is an antibody that specifically binds a subject polypeptide. Such antibodies are useful in diagnostic assays, e.g., to detect the presence of a subject polypeptide.

The further aspect of the present invention is a method of identifying an agent that modulates the level of a subject polypeptide (or an mRNA encoding a subject polypeptide) in a cell. The method generally involves contacting a cell (e.g., a eukaryotic host cell) that produces the subject polypeptide with a test agent; and determining the effect, if any, of the test agent on the level of the polypeptide in the cell.

The further aspect of the present invention is a method of identifying an agent that modulates the activity of a subject polypeptide. The methods generally involve contacting a subject polypeptide with a test agent; and determining the effect, if any, of the test agent on the activity of the polypeptide.

The further aspect of the present is biologically active agents screened and identified using a method of the invention. The further aspect of the present invention is a pharmaceutical composition comprising a subject agent; and a pharmaceutically acceptable excipient.

In another aspect, the invention provides a library of polynucleotides, wherein at least one of the polynucleotides comprises the sequence information of a polynucleotide of the invention. In specific embodiments, the library is provided on a nucleic acid array. In some embodiments, the library is provided in a computer-readable format.

The present inventions are as follows;

1. The isolated polynucleotide having a nucleotide sequence of a clone selected from the group consisting of 1110005I17 (SEQ ID NO: 1), 1700007F22 (SEQ ID NO: 2), 1700011J22 (SEQ ID NO: 3), 1700056N09 (SEQ ID NO: 4), 2310014H11 (SEQ ID NO: 5), 2310031C01 (SEQ ID NO: 6), 4930563B01 (SEQ ID NO: 7), 9130004I05 (SEQ ID NO: 8), 9230110A19 (SEQ ID NO: 9), 9230111O07 (SEQ ID NO: 10), A030004E11 (SEQ ID NO: 11), A430045L05 (SEQ ID NO: 12), A530065I17 (SEQ ID NO: 13), A830010B16 (SEQ ID NO:14), B230114O10 (SEQ ID NO: 15), B230352O20 (SEQ ID NO: 16), C230071E12 (SEQ ID NO: 17), C630041L24 (SEQ ID NO: 18) and D630020P16 (SEQ ID NO: 19),

2. The isolated polynucleotide according to claim 1 having a nucleotide sequence of a clone selected from the group consisting of 1700007F22 (SEQ ID NO: 2), A030004E11 (SEQ ID NO: 11), A530065I17 (SEQ ID NO: 13), B230352O20 (SEQ ID NO: 16), C630041L24 (SEQ ID NO: 18) and D630020P16 (SEQ ID NO: 19),

3. The isolated polypeptide having an amino acid sequence selected from the group consisting of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37 and SEQ ID NO:38, wherein the polypeotides having the amino acid sequence of SEQ ID NOs: 20 to 38 are corresponding to the polynucleotides having the nucleotide sequence of SEQ ID NOs: 1 to 19, respectively, and

4. The isolated polypeptide according to claim 4 having an amino acid sequence selected from the group consisting of SEQ ID NO:21, SEQ ID NO:30, SEQ ID NO:32, SEQ ID NO:35, SEQ ID NO:36 and SEQ ID NO:38.

### BRIEF DESCRIPTION OF THE DRAWINGS

FIG. 1 is a diagram of agarose gel electrophoresis of RT-PCR products from RNA derived from a small intestine of mouse using primers of various clones in Tables 3-1 to 3-3.

FIG. 2 is a diagram of agarose gel electrophoresis of RT-PCR products from RNA derived from a small intestine of mouse using primers of various clones in Tables 3-1 to 3-3.

FIG. 3 is a diagram of agarose gel electrophoresis of RT-PCR products from RNA derived from a stomach of mouse using primers of various clones in Tables 3-1 to 3-3.

FIG. 4 is a diagram of agarose gel electrophoresis of RT-PCR products from RNA derived from a small intestine of mouse using primers of various clones in Tables 3-1 to 3-3.

FIG. 5 is a diagram of agarose gel electrophoresis of RT-PCR products from RNA derived from a kidney of mouse using primers of various clones in Tables 3-1 to 3-3.

FIG. 6 is a diagram of agarose gel electrophoresis of RT-PCR products from RNA derived from a kidney of mouse using primers of various clones in Tables 3-1 to 3-3.

FIG. 7 is a diagram of agarose gel electrophoresis of RT-PCR products from RNA derived from a tangue of mouse using primers of various clones in Tables 3-1 to 3-3.

FIG. 8 is a diagram of agarose gel electrophoresis of RT-PCR products from RNA derived from a tangue of mouse using primers of various clones in Tables 3-1 to 3-3.

FIG. 9 is a diagram of agarose gel electrophoresis of RT-PCR products from RNA derived from a testis of mouse using primers of various clones in Tables 3-1 to 3-3.

FIG. 10 is a diagram of agarose gel electrophoresis of RT-PCR products from RNA derived from a testis of mouse using primers of various clones in Tables 3-1 to 3-3.

FIG. 11 is a diagram of agarose gel electrophoresis of RT-PCR products from RNA derived from an epididymis of mouse using primers of various clones in Tables 3-1 to 3-3.

FIG. 12 is a diagram of agarose gel electrophoresis of RT-PCR products from RNA derived from an epididymis of mouse using primers of various clones in Tables 3-1 to 3-3.

FIG. 13 is a diagram of agarose gel electrophoresis of RT-PCR products from RNA derived from an aorate and vein of mouse using primers of various clones in Tables 3-1 to 3-3.

FIG. 14 is a diagram of agarose gel electrophoresis of RT-PCR products from RNA derived from an aorate of mouse using primers of various clones in Tables 3-1 to 3-3.

FIG. 15 is a diagram of agarose gel electrophoresis of RT-PCR products from RNA derived from a thymus of mouse using primers of various clones in Tables 3-1 to 3-3.

FIG. 16 is a diagram of agarose gel electrophoresis of RT-PCR products from RNA derived from a thymus of mouse using primers of various clones in Tables 3-1 to 3-3.

FIG. 17 is a diagram of agarose gel electrophoresis of RT-PCR products from RNA derived from a heart of mouse using primers of various clones in Tables 3-1 to 3-3.

FIG. 18 is a diagram of agarose gel electrophoresis of RT-PCR products from RNA derived from a heart of mouse using primers of various clones in Tables 3-1 to 3-3.

FIG. 19 is a diagram of agarose gel electrophoresis of RT-PCR products from RNA derived from a liver of mouse using primers of various clones in Tables 3-1 to 3-3.

FIG. 20 is a diagram of agarose gel electrophoresis of RT-PCR products from RNA derived from a liver of mouse using primers of various clones in Tables 3-1 to 3-3.

FIG. 21 is a diagram of agarose gel electrophoresis of RT-PCR products from RNA derived from a skin of mouse using primers of various clones in Tables 3-1 to 3-3.

FIG. 22 is a diagram of agarose gel electrophoresis of RT-PCR products from RNA derived from a skin of mouse using primers of various clones in Tables 3-1 to 3-3.

FIG. 23 is a diagram of agarose gel electrophoresis of RT-PCR products from RNA derived from a pancreas of mouse using primers of various clones in Tables 3-1 to 3-3.

FIG. 24 is a diagram of agarose gel electrophoresis of RT-PCR products from RNA derived from a pancreas of mouse using primers of various clones in Tables 3-1 to 3-3.

FIG. 25 is a diagram of agarose gel electrophoresis of RT-PCR products from RNA derived from a cecum of mouse using primers of various clones in Tables 3-1 to 3-3.

FIG. 26 is a diagram of agarose gel electrophoresis of RT-PCR products from RNA derived from a cecum of mouse using primers of various clones in Tables 3-1 to 3-3.

FIG. 27 is a diagram of agarose gel electrophoresis of RT-PCR products from RNA derived from a hypothalamus of mouse using primers of various clones in Tables 3-1 to 3-3.

FIG. 28 is a diagram of agarose gel electrophoresis of RT-PCR products from RNA derived from a hypothalamus of mouse using primers of various clones in Tables 3-1 to 3-3.

FIG. 29 is a diagram of agarose gel electrophoresis of RT-PCR products from RNA derived from an olfactory-bulb of mouse using primers of various clones in Tables 3-1 to 3-3.

FIG. 30 is a diagram of agarose gel electrophoresis of RT-PCR products from RNA derived from an olfactory-bulb of mouse using primers of various clones in Tables 3-1 to 3-3.

FIG. 31 is a diagram of agarose gel electrophoresis of RT-PCR products from RNA derived from a cortex of mouse using primers of various clones in Tables 3-1 to 3-3.

FIG. 32 is a diagram of agarose gel electrophoresis of RT-PCR products from RNA derived from a cortex of mouse using primers of various clones in Tables 3-1 to 3-3.

FIG. 33 is a diagram of agarose gel electrophoresis of RT-PCR products from RNA derived from a hippocampus of mouse using primers of various clones in Tables 3-1 to 3-3.

FIG. 34 is a diagram of agarose gel electrophoresis of RT-PCR products from RNA derived from a hippocampus of mouse using primers of various clones in Tables 3-1 to 3-3.

FIG. 35 is a diagram of agarose gel electrophoresis of RT-PCR products from RNA derived from a c.qradrogemi of mouse using primers of various clones in Tables 3-1 to 3-3.

FIG. 36 is a diagram of agarose gel electrophoresis of RT-PCR products from RNA derived from a c.qradrogemi of mouse using primers of various clones in Tables 3-1 to 3-3.

FIG. 37 is a diagram of agarose gel electrophoresis of RT-PCR products from RNA derived from a pit.1 of mouse using primers of various clones in Tables 3-1 to 3-3.

FIG. 38 is a diagram of agarose gel electrophoresis of RT-PCR products from RNA derived from a pit.1 of mouse using primers of various clones in Tables 3-1 to 3-3.

FIG. 39 is a diagram of agarose gel electrophoresis of RT-PCR products from RNA derived from a diencepharose of mouse using primers of various clones in Tables 3-1 to 3-3.

FIG. 40 is a diagram of agarose gel electrophoresis of RT-PCR products from RNA derived from a diencepharose of mouse using primers of various clones in Tables 3-1 to 3-3.

FIG. 41 is a diagram of agarose gel electrophoresis of RT-PCR products from RNA derived from a c.striatum of mouse using primers of various clones in Tables 3-1 to 3-3.

FIG. 42 is a diagram of agarose gel electrophoresis of RT-PCR products from RNA derived from a c.striatum of mouse using primers of various clones in Tables 3-1 to 3-3.

FIG. 43 is a diagram of agarose gel electrophoresis of RT-PCR products from RNA derived from an m.oblongatu of mouse using primers of various clones in Tables 3-1 to 3-3.

FIG. 44 is a diagram of agarose gel electrophoresis of RT-PCR products from RNA derived from an m.oblongatu of mouse using primers of various clones in Tables 3-1 to 3-3.

FIG. 45 is a diagram of agarose gel electrophoresis of RT-PCR products from RNA derived from a cerebellum of mouse using primers of various clones in Tables 3-1 to 3-3.

FIG. 46 is a diagram of agarose gel electrophoresis of RT-PCR products from RNA derived from a cerebellum of mouse using primers of various clones in Tables 3-1 to 3-3.

FIG. 47 is a diagram of agarose gel electrophoresis of RT-PCR products from RNA derived from a lung of mouse using primers of various clones in Tables 3-1 to 3-3.

FIG. 48 is a diagram of agarose gel electrophoresis of RT-PCR products from RNA derived from a lung of mouse using primers of various clones in Tables 3-1 to 3-3.

FIG. 49 is a diagram of in vitro transcription/translation pattern for clone Nos. C230071E12, 9230111O07 and 1700056N09.

FIG. 50 is a diagram of in vitro transcription/translation pattern for clone Nos. A430045L05, A030004E11 and B230352O20.

FIG. 51 is a diagram of in vitro transcription/translation pattern for clone Nos. C630041L24, 2310031C01, 1110005I17 and 2310014H11.

FIG. 52 is a diagram of in vitro transcription/translation pattern for clone Nos. A530065I17, 1700011J22, 9130004I05 and B230114O10.

### DETAILED DESCRIPTION OF THE INVENTION

The present invention provides novel isolated polypeptides, and compositions comprising same. The present invention provides novel isolated polynucleotides encoding the subject polypeptides, as well as recombinant vectors and host cells comprising same. The present invention provides methods of producing a subject polypeptide. The present invention provides antibodies that specifically bind to a subject polypeptide. The present invention further provides screening methods for identifying agents that modulate a level or an activity of a subject polypeptide. The present invention provides agents that modulate a level or an activity of a subject polypeptide, as well as compositions, including pharmaceutical compositions, comprising a subject agent.

In the present specification, the terms are defined as follows.

The terms “polynucleotide,” “nucleic acid,” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, doublestranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers.

Polynucleotides include splice variants of an mRNA. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The áanomer of deoxyribose may be used, where the base is inverted with respect to the natural â-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “substantially isolated” or “isolated” polynucleotide is one that is substantially free of the sequences with which it is associated in nature. By substantially free is meant at least 50%, preferably at least 70%, more preferably at least 80%, and even more preferably at least 90% free of the materials with which it is associated in nature. As used herein, an “isolated” polynucleotide also refers to recombinant polynucleotides, which, by virtue of origin or manipulation: (1) are not associated with all or a portion of a polynucleotide with which it is associated in nature, (2) are linked to a polynucleotide other than that to which it is linked in nature, or (3) does not occur in nature.

Hybridization reactions can be performed under conditions of different “stringency”. Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where 1×SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water.

An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

For microarray-based hybridization, standard “high stringency conditions” are generally hybridization in 50% formamide, 5×SSC, 0.2 μg/μl poly(dA), 0.2 μg/μl human cot1 DNA, and 0.5% SDS, in a humid oven at 42° C. overnight, followed by successive washes of the microarray in 1×SSC, 0.2% SDS at 55° C. for 5 minutes, followed by washing at 0.1×SSC, 0.2% SDS at 55° C. for 20 minutes. For microarraybased hybridization, “moderate stringency conditions,” suitable for cross-hybridization to mRNA encoding structurally and functionally related proteins, are the same as those for high stringency conditions but with reduction in temperature for hybridization and washing to room temperature (e.g., about 22° C. to 25° C.).

As used herein, the term “alternative splicing” and related terms refers to all types of RNA processing that lead to expression of plural protein isoforms from a single gene. Accordingly, the term “splice variant” refers to mRNAs transcribed from a given gene that, however processed, collectively encode plural protein isoforms. For example, splice variants can include exon insertions, exon extensions, exon truncations, exon deletions, alternative in the 5′ untranslated region and alternatives in the 3′ untranslated region.

Stringent hybridization conditions are hybridization conditions that are at least as stringent as the above representative conditions. Other stringent hybridization conditions are known in the art and may also be employed to identify nucleic acids of this particular embodiment of the invention.

“Tm” is the temperature in degrees Celsius at which 50% of a polynucleotide duplex made of complementary strands hydrogen bonded in anti-parallel direction by Watson-Crick base pairing dissociates into single strands under conditions of the experiment. Tm may be predicted according to a standard formula, such as:

Tm=81.5+16.6log[X+]+0.41(% G/C)−0.61(% F)−600/L

where [X+] is the cation concentration (usually sodium ion, Na+) in mol/L; (% G/C) is the number of G and C residues as a percentage of total residues in the duplex; (% F) is the percent formamide in solution (wt/vol); and L is the number of nucleotides in each strand of the duplex.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at http://ww.ncbi.nlm.nih.gov/BLAST/.

Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:

Mismatch Penalty: 1.00;

Gap Penalty: 1.00;

Gap Size Penalty: 0.33; and

Joining Penalty: 30.0.

One parameter for determining percent sequence identity is the “percentage of the alignment region length” where the strongest alignment is found.

The percentage of the alignment region length is calculated by counting the number of residues of the individual sequence found in the region of strongest alignment. This number is divided by the total residue length of the target or query polynucleotide sequence to find a percentage. An example is shown below:

| Target sequence: | G C G C G A A A T A C T C A C T C G A G G |
|---|---|
| | \| \| \| \| \| \| \| \| \| \| \| |
| Query sequence: | T A T A G C C C T A C . C A C T A G A G T C C |
| | 1 5 1 0 1 5 |

The region of alignment begins at residue 9 and ends at residue 19. The total length of the target sequence is 20 residues. The percent of the alignment region length is 11 divided by 20 or 55%, for example.

Percent sequence identity is calculated by counting the number of residue matches between the target and query polynucleotide sequence and dividing total number of matches by the number of residues of the target or query sequence found in the region of strongest alignment. For the example above, the percent identity would be 10 matches divided by 11 residues, or approximately, 90.9%.

The percent of the alignment region length is typically at least about 55% of total length of the sequence, more typically at least about 58%, and even more typically at least about 60% of the total residue length of the sequence. Usually, percent length of the alignment region can be as great as about 62%, more usually as great as about 64% and even more usually as great as about 66%.

Stringent conditions for both DNA/DNA and DNA/RNA hybridization are as described by Sambrook et al. Molecular Cloning, A Laboratory Manual, 2nd Ed., Cold Spring Harbor Laboratory Press, Cold Spring Harbor, N.Y., 1989, herein incorporated by reference. For example, see page 7.52 of Sambrook et al.

The term “degenerate variant” of a reference nucleic acid sequence refers to all nucleic acid sequences that can be directly translated, using the standard genetic code, to provide an amino acid sequence identical to that translated from the reference nucleic acid sequence.

As used herein, the term “microarray” and the equivalent term “nucleic acid microarray” refer to a substrate-bound collection of plural nucleic acid, hybridization to each of the plurality of bound nucleic acids being separately detectable. The substrate can be porous or solid, planar or non-planar, unitary or distributed. As such, the term “microarray” includes all of the devices referred to as microarrays in Schena, ed. DNA Microarrays: A Practical Approach Oxford Univ. Press (1999); Nature Genetics 21:1-60 (1999) and Schena (ed.) Microarray Biochip: Tools and Technology Eaton Publishing Co./BioTechniques Books Division (2000); and Brenner et al. (2000) Proc. Natl. Acad. Sci. USA 97:1665-1670.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells tranfected or infected in vivo or in vitro with a recombinant vector or a polynucleotide of the invention. A host cell which comprises a recombinant vector of the invention is a “recombinant host cell.”

The term “binds specifically,” in the context of antibody binding, refers to high avidity and/or high affinity binding of an antibody to a specific polypeptide i.e., epitope of a subject polypeptide. Antibody binding to an epitope on a specific subject polypeptide is preferably stronger than binding of the same antibody to any other epitope, particularly those which may be present in molecules in association with, or in the same sample, as the specific polypeptide of interest, e.g., binds more strongly to a specific epitope than to a different epitope so that by adjusting binding conditions the antibody binds almost exclusively to the specific epitope and not to any other epitope on the same polypeptide, and not to any other polypeptide which does not comprise the epitope. Antibodies which bind specifically to a subject polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, antibodies of the invention which bind to a specific polypeptide with a binding affinity of 10⁻⁷ M or more, preferably 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines, simians, humans, felines, canines, equines, bovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent o those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

(1) Nucleic Acids

The present inventions provides novel nucleic acids encoding novel short polypeptides having various functions, which are subject polypeptides shown in the figure, or fragments thereof. By nucleic acid is meant a nucleic acids comprising a sequence of DNA having an open reading frame that encodes one the subject polypeptide and is capable, under appropriate conditions, of being expressed as one of the subject polypeptide described above. Thus, the term encompasses genomic DNA, cDNA, mRNA, splice variants, antisense RNA, RNAi, DNA comprising one or more single-nucleotide polymorphisms, and vectors comprising the subject nucleic acid sequences. Also encompassed in this term are nucleic acids that are homologous or substantially similar or identical to the nucleic acids encoding the subject proteins. Thus, the subject invention provides genes encoding a subject protein, and homologs thereof.

Mouse full-length cDNAs are shown in the Sequence listing.

A subject nucleic acid includes single nucleotide polymorphisms. Single nucleotide polymorphisms (SNPs) occur frequently in eukaryotic genomes. Nature (2001) 409:860-921. The nucleotide sequence determined from one individual of a species may differ from other allelic forms present within the population. The present invention encompasses such SNPs.

In some embodiments, a polynucleotide of the invention comprises a nucleotide sequence of at least about 30, at least about 50, at least about 75, at least about 100, at least about 150, at least about 200, at least about 300, at least about 400, at least about 500, at least about 600, at least about 700, at least about 800, at least about 900, or at least about 1000 contiguous nucleotides of any one of the sequences shown in the Sequence listing, or the coding region thereof, or a complement thereof.

In some embodiments, a polynucleotide of the invention has at least about 70%, at least about 75%, at least about 80%, at least about 85%, at least about 90%, at least about 95%, at least about 97%, at least about 98%, or at least about 99%, nucleotide sequence identity with a nucleotide sequence of the coding region of any one of the sequences shown in the Sequence listing, or a complement thereof. These sequence variants include naturally-occurring variants (e.g., SNPs, homologs from other species, and the like), and variants resulting from random or directed mutagenesis.

In some embodiments, a polynucleotide of the invention hybridizes under stringent hybridization conditions to a polynucleotide having the coding region of any one of the sequences shown in the Sequence listing, or the complement thereof.

In other embodiments, a polynucleotide of the invention comprises a nucleotide sequence that encodes a polypeptide comprising an amino acid sequence of at least about 10, at least about 20, at least about 25, at least about 30, at least about 40, at least about 50, at least about 60, at least about 70, at least about 75, at least about 80, at least about 90, or at least about 100 contiguous amino acids of one of the sequences shown in the Sequence listing (e.g., a polypeptide encoded by any one of the nucleotide sequences shown in the Sequence listing), up to the entire amino acid sequence as shown in the Sequence listing (or as encoded by any one of the nucleotide sequences shown in the Sequence listing).

The subject polynucleotides include those that encode variants of the polypeptides encoded by the sequences shown in the Sequence listing. Thus, in some embodiments, a subject polynucleotide encodes variant polypeptides that include insertions, deletions, or substitutions compared with the polypeptides encoded by the nucleotide sequences shown in the Sequence listing. Conservative amino acid substitutions include serine/threonine, valine/leucine/isoleucine, asparagine/histidine/glutamine, glutamic acid/aspartic acid, etc. See, e.g., Gonnet et al. (1992) Science 256:1443-1445.

The source of homologous genes may be any species, e.g., primate species, particularly human; rodents, such as rats and mice, canines, felines, bovines, ovines, equines, yeast, nematodes, etc. Between mammalian species, e.g., human and mouse, homologs have substantial sequence similarity, e.g. at least 60% sequence identity, usually at least 75%, more usually at least 80% between nucleotide sequences. In many embodiments of interest, homology will be at least 75, usually at least 80 and more usually at least 85%, where in certain embodiments of interest homology will be as high as 90%, 95%, 97%, 98%, or 99%. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings). The sequences provided herein are essential for recognizing related and homologous proteins in database searches.

Nucleic acids encoding the proteins and polypeptides of the subject invention may be cDNA or genomic DNA or a fragment thereof. The term gene shall be intended to mean the open reading frame encoding specific proteins and polypeptides of the subject invention, and introns, as well as adjacent 5′ and 3′ non-coding nucleotide sequences involved in the regulation of expression, up to about 20 kb beyond the coding region, but possibly further in either direction. The gene may be introduced into an appropriate vector for extrachromosomal maintenance or for integration into a host genome.

The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a protein according to the subject invention.

A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The nucleic acids of the subject invention may encode all or a part of the subject proteins. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt.

Subject nucleic acids that encode at least 8 contiguous amino acids (i.e., fragments of 24 nucleotides or more) are useful in directing the expression or the synthesis of peptides that have utility as immunogens. See, e.g., Lerner (1982) Nature 299:592-596; Shinnick et al. (1983) Ann. Rev. Microbiol. 37:425-446; and Sutcliffe et al. (1983) Science 219:660-666.

The present inventions also encompass nucleic acid compositions in which one or more of the nucleic acid of the present inventions are contained.

Nucleic acid molecules of the invention may comprise nucleic acid molecules other than the subject nucleic acid molecules (“heterologous nucleic acid molecules”) of any length. For example, the subject nucleic acid molecules may be flanked on the 5′ and/or 3′ ends by heterologous nucleic acid molecules of from about 1 nt to about 10 nt, from about 10 nt to about 20 nt, from about 20 nt to about 50 nt, from about 50 nt to about 100 nt, from about 100 nt to about 250 nt, from about 250 nt to about 500 nt, or from about 500 nt to about 1000 nt, or more in length. For example, when used as a probe to detect nucleic acid molecules capable of hybridizing with the subject nucleic acids, the subject nucleic acid molecules may be flanked by heterologous sequences of any length.

When used as probes, a subject nucleic acid may include nucleotide analogs that incorporate labels that are directly detectable, such as radiolabels or fluorophores, or nucleotide analogs that incorporate labels that can be visualized in a subsequent reaction, such as biotin or various haptens.

Common radiolabeled analogs include those labeled with 32P or 35S, such as α-32P-dATP, -dTTP, -dCTP, and dGTP; and α-35S-GTP, α35S-dATP, and the like.

Commercially available fluorescent nucleotide analogs readily incorporated into a subject nucleic acid include deoxyribonucleotides and/or ribonucleotide analogs labeled with Cy3, Cy5, Texas Red, Alexa Fluor dyes, rhodamine, cascade blue, BODIPY, and the like.

Haptens that are commonly conjugated to nucleotides for subsequent labeling include biotin, digoxigenin, and dinitrophenyl.

The subject nucleic acid compositions include antisense RNA, ribozymes, and RNAi, which are described in more detail below.

The nucleic acids of the invention can be used for antisense inhibition of transcription or translation, as described below. See, e.g., Phillips (ed.) Antisense Technology, Part B Methods in Enzymology Vol. 314, Academic Press, Inc. (1999); Phillips (ed.) Antisense Technology, Part A Methods in Enzymology Vol. 313, Academic Press, Inc. (1999); Hartmann et al. (eds.) Manual of Antisense Methodology (Perspectives in Antisense Science) Kluwer Law International (1999); Stein et al. (eds.) Applied Antisense Oligonucleotide Technology Wiley-Liss (1998); Agrawal et al. (eds) Antisense Research and Applications Springer-Verlag New York, Inc. (1998).

Nucleic acids of the invention can also be bound to a substrate. The substrate can be porous or solid, planar or non-planar, unitary or distributed; and the bond between the nucleic acid and the substrate can be covalent or non-covalent.

Substrates include, but are not limited to, a membrane, such as nitrocellulose, nylon, positively-charged derivatized nylon; a solid substrate such as glass, amorphous silicon, crystalline silicon, plastics (including e.g., polymethylacrylic, polyethylene, polypropylene, polyacrylate, polymethylmethacrylate, polyvinylchloride, polytetrafluoroethylene, polystyrene, polycarbonate, polyacetal, polysulfone, cellulose acetate, or mixtures thereof).

Subject nucleic acids can be attached covalently attached to a surface of the support or applied to a derivatized surface in a chaotropic agent that facilitates denaturation and adherence, e.g., by noncovalent interactions, or some combination thereof.

The nucleic acids can be bound to a substrate to which a plurality of other nucleic acids are concurrently bound, hybridization to each of the plurality of the bound nucleic acids being separately detectable.

The isolated nucleic acids of the invention can be used as probes to detect an characterize gross alteration in a genomic locus, such as deletions, insertions, translocations, and duplications, e.g., using fluorescence in situ hybridization (FISH) to chromosome spreads. See, e.g., Andreeff et al. (eds) Introduction to Fluorescence In Situ Hybridization: Principles and Clinical Applications, John Wiley & Sons (1999).

The subject nucleic acids are also useful to detect smaller genomic alterations, such as deletions, insertions, translocations, and substitutions (e.g., SNPs).

The subject nucleic acid molecules may also be provided as part of a vector (e.g., a polynucleotide construct), a wide variety of which are known in the art and need not be elaborated upon herein. Vectors include, but are not limited to, plasmids; cosmids; viral vectors; human, yeast, bacterial, and P1-derived artificial chromosomes (HAC's, YAC's, BAC's, PAC's, etc.); mini-chromosomes; and the like. Vectors are amply described in numerous publications well known to those in the art, including, e.g., Short Protocols in Molecular Biology, (1999) F. Ausubel, et al., eds., Wiley & Sons; Jones et al. (eds.) Vectors: Cloning Applications: Essential Techniques John Wiley & Son Ltd (1998); Jones et al. (eds.) Vectors: Expression Systems: Essential Techniques John Wiley & Son Ltd (1998). Vectors may provide for expression of the subject nucleic acids; may provide for propagating the subject nucleic acids, or both.

Where a subject nucleic acid is part of a vector, the vector is referred to as a “recombinant vector” or a “construct.” Subject constructs are useful for propagating a subject nucleic acid in a host cell (“cloning vectors”); for shuttling a subject nucleic acid between host cells derived from disparate organisms (“shuttle vectors”); for inserting a subject nucleic acid into a host cell's chromosome (“insertion vectors”); for expressing sense or antisense RNA transcripts of the invention in vitro (e.g., in a cell-free system or within an in vitro cultured host cell) (“expression vectors”); and for producing a subject polypeptide encoded by a subject nucleic acid (“expression vectors”).

Vectors typically include at least one origin of replication, at least one site for insertion of heterologous nucleic acid (e.g., in the form of a polylinker with multiple, tightly clustered, single cutting restriction endonuclease recognition sites), and at least one selectable marker, although some integrative vectors will lack an origin that is functional in the host to be chromosomally modified, and some vectors will lack selectable markers.

The subject genes are isolated and obtained in substantial purity, generally as other than an intact chromosome. Usually, the DNA will be obtained substantially free of other nucleic acid sequences that do not include a sequence or fragment thereof of the subject genes, generally being at least about 50%, usually at least about 90% pure and are typically “recombinant”, i.e. flanked by one or more nucleotides with which it is not normally associated on a naturally occurring chromosome.

The present inventions also provides isolated primer pairs.

In some embodiments, the invention provides isolated nucleic acids that, when used as primers in a polymerase chain reaction, amplify a subject polynucleotide, or a polynucleotide containing a subject polynucleotide. The amplified polynucleotide is from about 20 to about 50, from about 50 to about 75, from about 75 to about 100, from about 100 to about 125, from about 125 to about 150, from about 150 to about 175, from about 175 to about 200, from about 200 to about 250, from about 250 to about 300, from about 300 to about 350, from about 350 to about 400, from about 400 to about 500, from about 500 to about 600, from about 600 to about 700, from about 700 to about 800, from about 800 to about 900, from about 900 to about 1000, from about 1000 to about 2000, from about 2000 to about 3000, from about 3000 to about 4000, from about 4000 to about 5000, or from about 5000 to about 6000 nucleotides or more in length. The isolated nucleic acids that, when used as primers in a polymerase chain reaction, amplify a polynucleotide, are from about 10 to about 20, from about 20 to about 30, from about 30 to about 40, from about 40 to about 50, from about 50 to about 100, or from about 100 to about 200 nucleotides in length. Generally, the nucleic acids are used in pairs in a polymerase chain reaction, where they are referred to as “forward” and “reverse” primers.

Thus, in some embodiments, the invention provides a pair of isolated nucleic acid molecules, each from about 10 to 200 nucleotides in length, the first nucleic acid molecule of the pair comprising a sequence of at least 10 contiguous nucleotides having 100% sequence identity to a nucleic acid sequence as shown in the Sequence listing and the second nucleic acid molecule of the pair comprising a sequence of at least 10 contiguous nucleotides having 100% sequence identity to the reverse complement of the nucleic acid sequence shown in the Sequence listing, wherein the sequence of the second nucleic acid molecule is located 3′ of the nucleic acid sequence of the first nucleic acid molecule shown in the Sequence listing. The primer nucleic acids are prepared using any known method, e.g., automated synthesis, and the like.

In some embodiments, the first and/or the second nucleic acid molecules comprises a detectable label. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. 32P, 35S, 3H; etc. The label may be a two stage system, where the amplified DNA is conjugated to biotin, haptens, etc. having a high affnity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The invention further provides a kit comprising a pair of nucleic acids as described above. The nucleic acids are present in a suitable storage medium, e.g., buffered solution, typically in a suitable container. The kit includes the pair of nucleic acids, and may further include a buffer; reagents for polymerase chain reaction (e.g., deoxynucleotide triphosphates (dATP, dTTP, dCTP, and dGTP), a thermostable DNA polymerase, a buffer suitable for polymerase chain reaction, a solution containing Mg²⁺ ions (e.g., MgCl₂), and other components well known to those skilled in the art for carrying out a polymerase chain reaction). The kit may further include instructions for use of the kit, which instructions may be provided in a variety of forms, e.g., as printed information, on a compact disc, and the like. The kit may further include reagents necessary for extraction of DNA from a biological sample (e.g., biopsy sample, blood, and the like) from an individual. The kits are useful in diagnostic applications, as described in more detail below. For example, the kit is useful to determine whether a given DNA sample isolated from an individual, comprises an expressed subject nucleic acid, a polymorphism, etc.

The polynucleotides of the present invention are exemplified in Tables 3-1 to 3-3. Their nucleotide sequences are represented by SEQ ID NOs: 1 to 19.

Furthermore, the polynucleotides of the present inventions includes polynucleotides having nucleotide encoding signal peptide. It is deduced that the peptides encoded by these polynucleotides have an important function for a living body.

(2) Computer-Related Embodiments of the polypeptides

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in one cell compared to another (e.g., a first cell type compared to a second cell type; a normal cell compared to a diseased cell; a cell not exposed to a signal or stimulus compared to a cell exposed to that signal or stimulus; and the like).

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between, e.g., a first cell type compared to a second cell type (e.g., expression in a brain cell compared to expression in a kidney cell); a normal cell compared to a diseased cell (e.g., a non-cancerous cell compared to a cancerous cell); a cell not exposed to an internal or external signal or stimulus compared to a cell exposed to that signal or stimulus (e.g., a cell contacted with a ligand compared to a control cell not contacted with the ligand); and the like. Other combinations and comparisons of cells will be readily apparent to the ordinarily skilled artisan.

Biochemical embodiments of the library include a collection of nucleic acids that have the sequences of the genes in the library, where the nucleic acids can correspond to the entire gene in the library or to a fragment thereof, as described in greater detail below.

External and internal signals include, but are not limited to, infection of a cell by a microorganism, including, but not limited to, a bacterium (e.g., Mycobacterium spp., Shigella, Chlamydia, and the like), a protozoan (e.g., Trypanosoma spp., Plasmodium spp., Toxoplasma spp., and the like), a fungus, a yeast (e.g., Candida spp.), or a virus (including viruses that infect mammalian cells, such as human immunodeficiency virus, foot and mouth disease virus, Epstein-Barr virus, and the like; viruses that infect plant cells; etc.); change in pH of the medium in which a cell is maintained or a change in internal pH; excessive heat relative to the normal range for the cell or the multicellular organism; excessive cold relative to the normal range for the cell or the multicellular organism; an effector molecule such as a hormone, a cytokine, a chemokine, a neurotransmitter; an ingested or applied drug; a ligand for a cell-surface receptor; a ligand for a receptor that exists internally in a cell, e.g., a nuclear receptor; hypoxia; light; dark; sleep patterns; electrical charge; ion concentration of the medium in which a cell is maintained or an internal ion concentration, exemplary ions including sodium ions, potassium ions, chloride ions, calcium ions, and the like; presence or absence of a nutrient; metal ions; a transcription factor; mitogens, including, but not limited to, lipopolysaccharide (LPS), pokeweed mitogen; antigens; a tumor suppressor; cell-cell contact; and the like.

The polynucleotide libraries of the subject invention generally comprise sequence information of a plurality of polynucleotide sequences, where at least one of the polynucleotides has a sequence of any of the sequences shown in the sequence listing. By plurality is meant at least 2, usually at least 3 and can include up to all of the sequences shown in the sequence listing. The length and number of polynucleotides in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides shown in the sequence listing, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information is publicly available. For example, the gapped BLAST (Altschul et al. Nucleic Acids Res. (1997) 25:3389-3402) and BLAZE (Brutlag et al. Comp. Chem. (1993) 17:203) search algorithms on a Sybase system, or the TeraBLAST (TimeLogic, Crystal Bay, Nev.) program optionally running on a specialized computer platform available from TimeLogic, can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif, or expression levels of a polynucleotide in a sample, with the stored sequence information.

Search means can be used to identify fragments or regions of the genome that match a particular target sequence or target motif. A variety of known algorithms are publicly known and commercially available, e.g. MacPattern (EMBL), BLASTN and BLASTX (NCBI), TeraBLAST (TimeLogic, Crystal Bay, Nev.). A “target sequence” can be any polynucleotide or amino acid sequence of six or more contiguous nucleotides or two or more amino acids, preferably from about 10 to 100 amino acids or from about 30 to 300 nt A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but are not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention.

One format for an output means ranks the relative expression levels of different polynucleotides. Such presentation provides a skilled artisan with a ranking of relative expression levels to determine a gene expression profile.

As discussed above, the “library” of the invention also encompasses biochemical libraries of the polynucleotides shown in the sequence listing, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of the polynucleotide sequences shown in the sequence listing is represented on the array. By array is meant an article of manufacture that has at least a substrate with at least two distinct nucleic acid targets on one of its surfaces, where the number of distinct nucleic acids can be considerably higher, typically being at least 10, usually at least 20, and often at least 25 distinct nucleic acid molecules. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the polypeptides of the library will represent at least a portion of the polypeptides encoded by a gene corresponding to one or more of the sequences shown in the sequence listing.

(3) Polypeptides

Novel polypeptides, as well as polypeptide compositions related thereto, are provided. The invention provides a subject polypeptide present in other than its natural environment. Novel polypeptides of the invention encompass proteins encoded by the nucleic acids having nucleotide sequences shown in the sequence listing. In some embodiments, a subject polypeptide is a human polypeptide. In other embodiments, a subject polypeptide is a mouse polypeptide.

In particular embodiments, a subject polypeptide has an amino acid sequence substantially identical to the sequence of that of any polypeptide encoded by a nucleotide sequence shown in the sequence listing.

In many embodiments, a novel polypeptide of the invention comprises an amino acid sequence of less than 100 amino acids in length. In general, a polypeptide of the invention has a molecular weight of from about 0.5 to about 10 kDa based on the amino acid sequence. The actual molecular weight of a subject polypeptide produced by a cell may be higher due to glycosylation and/or other modifications. Furthermore, where a subject protein is a fusion protein comprising a subject secreted factor and a fusion partner, the subject protein may have a molecular weight greater than 10 kDa.

In many embodiments, a subject protein is secreted from a cell, e.g., a eukaryotic cell. In some of these embodiments, a subject protein is secreted into the extracellular space. In other embodiments, a subject protein is secreted, and becomes associated with the cell membrane. In other embodiments, a subject protein is secreted, and becomes associated with the extracellular matrix.

In many embodiments, a subject secreted protein has one or more of the following activities: (1) functions as a cellular differentiation factor; (2) affects an immune response; (3) functions as a cellular growth factor; (4) functions as a hormone; (5) modulates appetite; (6) affects an endocrine function; (7) functions as a cytokine; (8) functions as a chemokine; (9) functions as a cytotoxic factor; (10) functions to modulate angiogenesis; (11) functions as a vasodilator; (12) functions as a vasoconstrictor; (13) functions to modulate blood pressure; (14) functions to induce cell motility (e.g., chemoattractants).

In some embodiments, a subject secreted protein binds to a receptor. In some of these embodiments, the receptor is on the cell that secretes the subject protein. In other embodiments, the receptor is on a cell other than the cell that secretes the subject protein.

In other embodiments, a subject secreted protein binds to a macromolecule. In other embodiments, a subject secreted protein binds to a second factor and modulates the activity of the second factor. In other embodiments, a subject secreted protein binds to a second factor and inhibits binding of the second factor to a receptor.

The invention also provides fragments of the subject polypeptide. In some embodiments, fragments exhibit one or more activities associated with a corresponding naturally occurring polypeptide. Fragments find utility in generating antibodies to the full-length polypeptide; and in methods of screening for candidate agents that bind to and/or modulate polypeptide activity. The term “polypeptide composition” as used herein refers to both the full-length human or mouse protein as well as portions or fragments thereof. Also included in this term are variations of the naturally occurring human protein, where such variations are homologous or substantially similar to the naturally occurring protein, as described in greater detail below, as well as corresponding homologs from non-human species, such as other mammalian species. In the following description of the subject invention, the terms “polypeptide” are used to refer not only to the mouse and human forms of these novel polypeptide, but also to homologs thereof expressed in non-human species.

In some embodiments, a subject polypeptide is present as a multimer. Multimers include homodimers, homotrimers, homotetramers, and multimers that include more than four monomeric units. Multimers also include hetermultimers, e.g., heterodimers, heterotrimers, heterotetramers, etc. where the subject polypeptide is present in a complex with proteins other than the subject polypeptide (where a protein other than a subject protein is a “heterologous protein”). Where the multimer is a heteromultimer, the subject polypeptide may be present in a 1:1 ratio, a 1:2 ratio, a 2:1 ratio, or other ratio, with a heterologous protein.

In addition to the above specifically listed proteins, polypeptides from other species are also provided, including mammals, such as: rodents, e.g. mice, rats; domestic animals, e.g. horse, cow, dog, cat; and humans, as well as non-mammalian species, e.g. avian, and the like. By homolog is meant a protein having at least about 35%, at least about 40%, at least about 60%, at least about 70%, at least about 75%, at least about 80%, at least about 90%, or at least about 95%, or higher, amino acid sequence identity to the one of the above specifically listed polypeptide, as measured by using the “GAP” program (part of the Wisconsin Sequence Analysis Package available through the Genetics Computer Group, Inc. (Madison Wis.)), where the parameters are: Gap weight: 12; length weight: 4. In many embodiments of interest, homology will be at least 75, usually at least 80 and more usually at least 85%, where in certain embodiments of interest homology will be as high as 90%.

Also provided are polypeptides that are substantially identical to the above listed proteins, where by substantially identical is meant that the protein has an amino acid sequence identity to the sequence one of the above listed proteins of at least about 75%, at least about 80% at least about 85%, at least about 90%, at least about 95%, or at least about 98%.

The proteins of the subject invention (e.g. polypeptides encoded by the nucleotide sequences shown in the sequence listing) are present in a non-naturally occurring environment, e.g. are separated from their naturally occurring environment. In certain embodiments, the subject proteins are present in a composition that is enriched for subject protein as compared to its naturally occurring environment. For example, purified polypeptide are provided, where by purified is meant that the polypeptide is present in a composition that is substantially free of non-polypeptide proteins, where by substantially free is meant that less than 90%, usually less than 60% and more usually less than 50% of the composition is made up of non-polypeptide proteins. For example, a subject polypeptide is present in a composition wherein at least about 50%, at least about 60%, at least about 70%, at least about 80%, at least about 90%, at least about 95%, or at least about 99% of the total macromolecules (polypeptides, polynucleotides, lipids, polysaccharides, and the like) in the composition is a subject polypeptide.

The proteins of the subject invention may also be present as an isolate, by which is meant that the protein is substantially free of other proteins and other naturally occurring biologic molecules, such as oligosaccharides, polynucleotides and fragments thereof, and the like, where substantially free in this instance means that less than 70%, usually less than 60% and more usually less than 50% of the composition containing the isolated protein is some other naturally occurring biological molecule. In certain embodiments, the proteins are present in substantially pure form, where by substantially pure form is meant at least 95%, usually at least 97% and more usually at least 99% pure.

In addition to the naturally occurring proteins, polypeptides which vary from the naturally occurring proteins are also provided. By a subject polypeptide is meant an amino acid sequence encoded by an open reading frame (ORF) as shown in the Sequence listing, described in greater detail below, including the full length protein and fragments thereof, particularly biologically active fragments and/or fragments corresponding to functional domains, e.g., enzyme active site, a domain for interaction with other protein(s), a domain for binding DNA, a regulatory domain, etc.; and including fusions of the subject polypeptides to other proteins or parts thereof. Fusion proteins may comprise a subject polypeptide, or fragment thereof, and a polypeptide other than a subject polypeptide (“the fusion partner”) fused in-frame at the N-terminus and/or C-terminus of the subject polypeptide, or internally to the subject polypeptide.

Suitable fusion partners include, but are not limited to, polypeptides that can bind antibody specific to the fusion partner (e.g., epitope tags, e.g., hemagglutinin, FLAG, cmyc, and the like); polypeptides that provide a detectable signal (e.g., a fluorescent protein, e.g., a green fluorescent protein, a fluorescent protein from an Anthozoan species; α-galactosidase; luciferase; and the like); polypeptides that provide a catalytic function or induce a cellular response; polypeptides that provide for secretion of the fusion protein from a eukaryotic cell; polypeptides that provide for secretion of the fusion protein from a prokaryotic cell; polypeptides that provide for binding to metal ions (e.g., Hisn, where n=3-10, e.g., 6His); and the like.

For example, where the fusion partner provides an immunologically recognizable epitope (an “epitope tag”), an antibody specific for an epitope of the fusion partner can be used to detect and quantitate the level of polypeptide. In some embodiments, the fusion partner provides for a detectable signal, and in these embodiments, the detection method is chosen based on the type of signal generated by the fusion partner. For example, where the fusion partner is a fluorescent protein, fluorescence is measured.

Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like. Where the fusion partner is an enzyme that yields a detectable product, the product can be detected using an appropriate means, e.g., â-galactosidase can, depending on the substrate, yield colored product, which is detected spectrophotometrically, or a fluorescent product; luciferase can yield a luminescent product detectable with a luminometer; etc.

In some embodiments, a polypeptide of the invention comprises at least about 10, at least about 20, at least about 25, at least about 30, at least about 40, at least about 50, at least about 60, at least about 70, at least about 75, at least about 80, or at least about 90 contiguous amino acids of one of the sequences shown in the sequence listing (e.g., a polypeptide encoded by a nucleotide sequence shown in the sequence listing), up to the entire amino acid sequence of a sequence shown in the sequence listing.

Fragments of the subject polypeptides, as well as polypeptides comprising such fragments, are also provided. Fragments of polypeptide of interest will typically be at least about 10 amino acids (aa) in length, usually at least about 50 aa in length, and may be as long as 80 aa in length or longer, where the fragment will have a stretch of amino acids that is identical to the subject protein of at least about 10 aa, and usually at least about 15 aa, and in many embodiments at least about 50 aa in length.

Specific fragments of interest include those with enzymatic activity, fragments that bind to other proteins, fragments that bind to DNA, and the like.

The invention provides polypeptides comprising such fragments, including, e.g., fusion polypeptides comprising a subject polypeptide fragment fused in frame (directly or indirectly) to a heterologous protein. Suitable heterologous proteins include, but are not limited to, a protein that serves as a detectable marker (e.g., a fluorescent protein, α-galactosidase, luciferase); an immunologically detectable protein (e.g., an epitope tag); and a structural protein.

Polypeptide fragments, such as those described above, are useful in screening assays, to identify agents that modulate an activity of a subject polypeptide. Screening assays are described in more detail below.

The subject proteins and polypeptides may be obtained from naturally occurring sources or synthetically produced. Where obtained from naturally occurring sources, the source chosen will generally depend on the species from which the protein is to be derived. The subject proteins may also be derived from synthetic means, e.g. by expressing a recombinant gene encoding protein of interest in a suitable host, as described in greater detail below. Any convenient protein purification procedures may be employed, where suitable protein purification methodologies are described in Guide to Protein Purification, (Deuthser ed.) (Academic Press, 1990). For example, a lysate may be prepared from the original source and purified using HPLC, exclusion chromatography, gel electrophoresis, affinity chromatography, and the like.

The present inventions also encompass polypeptide compositions in which one or more of the polypeotides of the present inventions are contained.

The sequences of the polypeptides of the present invention are represented by SEQ ID NOs: 20 to 38.

(4) Preparation of the Subject Polypeptides; Host Cells

In addition to the plurality of uses described in greater detail in following sections, the subject nucleic acid compositions find use in the preparation of all or a portion of the polypeptides of the subject invention, as described above. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Thus, the instant invention provides methods of producing a subject polypeptide.

The methods generally involve introducing a subject construct into a host cell in vitro; and culturing the host cell in vitro under conditions that are suitable for expression of the construct and production of the encoded subject polypeptide; and harvesting the subject polypeptide, e.g., from the culture medium, from within the host cell (e.g., by disrupting the host cell), or both.

The instant invention also provides methods of producing a subject polypeptide using cell-free in vitro transcription/translation methods, which are well known in the art.

The instant invention further provides host cells, e.g., recombinant host cells, that comprise a subject nucleic acid, and host cells that comprise a subject recombinant vector. Subject host cells can be in in vitro culture, or may be part of a multicellular organism. Host cells are described in more detail below. The instant invention further provides transgenic, non-human animals, as described in more detail below.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, or any of the above-described fragment, and up to the complete open reading frame of the gene. After introduction of the DNA, the cells containing the construct may be selected by means of a selectable marker, the cells expanded and then used for expression.

Proteins and polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the gene in eukaryotic cells, where the encoded protein will benefit from native folding and posttranslational modifications. Small peptides can also be synthesized in the laboratory.

Polypeptides that are subsets of the complete sequences of the subject proteins may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Macda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence inserted into the genome of the cell at location sufficient to at least enhance expressed of the gene in the cell. The regulatory sequence may be designed to integrate into the genome via homologous recombination, as disclosed in U.S. Pat. Nos. 5,641,670 and 5,733,761, the disclosures of which are herein incorporated by reference, or may be designed to integrate into the genome via non-homologous recombination, as described in WO 99/15650, the disclosure of which is herein incorporated by reference. As such, also encompassed in the subject invention is the production of the subject proteins without manipulation of the encoding nucleic acid itself, but instead through integration of a regulatory sequence into the genome of cell that already includes a gene encoding the desired protein, as described in the above incorporated patent documents.

The subject proteins and polypeptides may be obtained from naturally occurring sources or synthetically produced. For example, the proteins may be derived from biological sources which express the proteins. The subject proteins may also be derived from synthetic means, e.g. by expressing a recombinant gene encoding protein of interest in a suitable host, as described in greater detail infra. Any convenient protein purification procedures may be employed, where suitable protein purification methodologies are described in Guide to Protein Purification, (Deuthser ed.) (Academic Press, 1990). For example, a lysate may prepared from the original source, (e.g. a cell expressing endogenous subject polypeptide, or a cell comprising the expression vector expressing the subject polypeptide(s)), and purified using HPLC, exclusion chromatography, gel electrophoresis, affinity chromatography, and the like.

(5) Antibodies Specific for a Polypeptide of the Invention

The invention provides antibodies that are specific for a subject polypeptide.

Suitable antibodies are obtained by immunizing a host animal with peptides comprising all or a portion of the target protein. Suitable host animals include mouse, rat sheep, goat, hamster, rabbit, etc. The origin of the protein immunogen may be mouse, human, rat, monkey etc. The host animal will generally be a different species than the immunogen, e.g. human protein used to immunize mice, etc.

The immunogen may comprise the complete protein, or fragments and derivatives thereof. Preferred immunogens comprise all or a part of one of the subject proteins, where these residues contain the post-translation modifications, such as glycosylation, found on the native target protein. Immunogens comprising the extracellular domain are produced in a variety of ways known in the art, e.g. expression of cloned genes using conventional recombinant methods, isolation from tumor cell culture supernatants, etc.

For preparation of polyclonal antibodies, the first step is immunization of the host animal with the target protein, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity.

Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using protein according to the subject invention bound to an insoluble support, protein A Sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Also provided are “artificial” antibodies, e.g., antibodies and antibody fragments produced and selected in vitro. In some embodiments, such antibodies are displayed on the surface of a bacteriophage or other viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

For in vivo use, particularly for injection into humans, it is desirable to decrease the antigenicity of the antibody. An immune response of a recipient against the blocking agent will potentially decrease the period of time that the therapy is effective. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521).

mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202).

Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

In yet other embodiments, the antibodies may be fully human antibodies. For example, xenogeneic antibodies which are identical to human antibodies may be employed. By xenogenic human antibodies is meant antibodies that are the same has human antibodies, i.e. they are fully human antibodies, with exception that they are produced using a non-human host which has been genetically engineered to express human antibodies. See e.g. WO 98/50433; WO 98,24893 and WO 99/53049, the disclosures of which are herein incorporated by reference.

Antibody fragments, such as Fv, F(ab′)2 and Fab may be prepared by cleavage of the intact protein, e.g. by protease or chemical cleavage. Alternatively, a truncated gene is designed. For example, a chimeric gene encoding a portion of the F(ab′)2 fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

(6) Compositions

The present invention further provides compositions, including pharmaceutical compositions, comprising the polypeptides, polynucleotides, antibodies, recombinant vectors, or host cells of the invention. These compositions may include a buffer, which is selected according to the desired use of the polypeptide, antibody, polynucleotide, recombinant vector, or host cell, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, A. Gennaro (1995) “Remington: The Science and Practice of Pharmacy”, 19th edition, Lippincott, Williams, & Wilkins.

(7) Uses of the Subject Polypeptides and Nucleic Acids

The subject polypeptides and nucleic acids find use in a variety of different applications, including research, diagnostic, and therapeutic agent screening/discovery/preparation applications, as well as therapeutic compositions.

General Applications

The subject nucleic acid compositions find use in a variety of different applications. Applications of interest include: the identification of homologs of the subject polypeptide; as a source of novel promoter elements; the identification of expression regulatory factors; as probes and primers in hybridization applications, e.g. polymerase chain reaction (PCR); the identification of expression patterns in biological specimens; the preparation of cell or animal models for function of the subject polypeptide; the preparation of in vitro models for function of the subject polypeptide; etc.

Homologs are identified by any of a number of methods. A fragment of the provided cDNA may be used as a hybridization probe against a cDNA library from the target organism of interest, where low stringency conditions are used. The probe may be a large fragment, or one or more short degenerate primers. Nucleic acids having sequence similarity are detected by hybridization under low stringency conditions, for example, at 50° C. and 6×SSC (0.9 M sodium chloride/0.09 M sodium citrate) and remain bound when subjected to washing at 55° C. in 1×SSC (0.15 M sodium chloride/0.015 M sodium citrate). Sequence identity may be determined by hybridization under stringent conditions, for example, at 50° C. or higher and 0.1×SSC (15 mM sodium chloride/1.5 mM sodium citrate). Nucleic acids having a region of substantial identity to the provided nucleic acid sequences, e.g. allelic variants, genetically altered versions of the gene, etc., bind to the provided sequences under stringent hybridization conditions. By using probes, particularly labeled probes of DNA sequences, one can isolate homologous or related genes.

The sequence of the 5′ flanking region may be utilized for promoter elements, including enhancer binding sites, that provide for developmental regulation in tissues where the subject genes are expressed. The tissue specific expression is useful for determining the pattern of expression, and for providing promoters that mimic the native pattern of expression. Naturally occurring polymorphisms in the promoter region are useful for determining natural variations in expression, particularly those that may be associated with disease.

Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, see Blackwell et al. (1995), Mol. Med. 1:194-205; Mortlock et al. (1996), Genome Res. 6:327-33; and Joulin and Richard-Foy (1995), Eur. J. Biochem. 232:620-626.

The regulatory sequences may be used to identify cis acting sequences required for transcriptional or translational regulation of expression, especially in different tissues or stages of development, and to identify cis acting sequences and trans-acting factors that regulate or mediate expression. Such transcription or translational control regions may be operably linked to a gene in order to promote expression of wild type or proteins of interest in cultured cells, or in embryonic, fetal or adult tissues, and for gene therapy.

Small DNA fragments are useful as primers for PCR, hybridization screening probes, etc. Larger DNA fragments, i.e. greater than 100 nt are useful for production of the encoded polypeptide, as described in the previous section. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages.

Amplification primers hybridize to complementary strands of DNA, and will prime towards each other.

The DNA may also be used to identify expression of the gene in a biological specimen. The manner in which one probes cells for the presence of particular nucleotide sequences, as genomic DNA or RNA, is well established in the literature.

Briefly, DNA or mRNA is isolated from a cell sample. The mRNA may be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of gene expression in the sample.

The sequence of a gene according to the subject invention, including flanking promoter regions and coding regions, may be mutated in various ways known in the art to generate targeted changes in promoter strength, sequence of the encoded protein, etc. The DNA sequence or protein product of such a mutation will usually be substantially similar to the sequences provided herein, i.e. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon. Other modifications of interest include epitope tagging, e.g. with the FLAG system, HA, etc. For studies of subcellular localization, fusion proteins with green fluorescent proteins (GFP) or other fluorescent proteins (e.g., those derived from Anthozoa species, derivatives of such proteins) may be used.

Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet. 199:537-9; and Prentki et al. (1984), Gene 29:303-13. Methods for site specific mutagenesis can be found in Sambrook et al., Molecular Cloning: A Laboratory Manual, CSH Press 1989, pp. 15.3-15.108; Weiner et al. (1993), Gene 126:35-41; Sayers et al. (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4. Such mutated genes may be used to study structure-function relationships of the subject proteins, or to alter properties of the protein that affect its function or regulation.

The subject nucleic acids can be used to generate transgenic, non-human animals and/or site-specific gene modifications in cell lines. Thus, in some embodiments, the invention provides a non-human transgenic animal comprising, as a transgene integrated into the genome of the animal, a nucleic acid molecule comprising a sequence encoding a subject polypeptide in operable linkage with a promoter, such that the subject polypeptide-encoding nucleic acid molecule is expressed in a cell of the animal.

Transgenic animals may be made through homologous recombination, where the endogenous locus is altered. Alternatively, a nucleic acid construct is randomly integrated into the genome. Vectors for stable integration include plasmids, retroviruses and other animal viruses, YACs, and the like.

Numerous publications are available that teach how to make transgenic animals, including, e.g., Transgenesis Techniques: Principles and Protocols D. Murphy and D. A. Carter, ed. (June 1993) Humana Press; Transgenic Animal Technology: A Laboratory Handbook C. A. Pinkert, ed. (January 1994) Academic Press; Transgenic Animals F. Grosveld and G Kollias, eds. (July 1992) Academic Press; and Embryonal Stem Cells: Introducing Planned Changes into the Animal Germline M. L. Hooper (January 1993) Gordon & Breach Science Pub.

The modified cells or animals are useful in the study of gene function and regulation. For example, a series of small deletions and/or substitutions may be made in the host's native gene to determine the role of different exons in oncogenesis, signal transduction, etc. Of interest is the use of genes to construct transgenic animal models for cancer, where expression of the subject protein is specifically reduced or absent.

Specific constructs of interest include anti-sense constructs, which will block expression, expression of dominant negative mutations, and over-expression of genes. Where a sequence is introduced, the introduced sequence may be either a complete or partial sequence of a gene native to the host, or may be a complete or partial sequence that is exogenous to the host animal, e.g., a human sequence of the subject invention. A detectable marker, such as lac Z may be introduced into the locus, where upregulation of expression will result in an easily detected change in phenotype.

One may also provide for expression of the gene, e.g. a subject gene, or variants thereof in cells or tissues where it is not normally expressed, at levels not normally present in such cells or tissues, or at abnormal times of development. One may also generate host cells (including host cells in transgenic animals) that comprise a heterologous nucleic acid molecule which encodes a polypeptide which functions to modulate expression of an endogenous promoter or other transcriptional regulatory region.

DNA constructs for homologous recombination will comprise at least a portion of the human gene or of a gene native to the species of the host animal, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990), Meth. Enzymol. 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny.

If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc., e.g. to determine the effect of a candidate drug on polypeptide activity.

Diagnostic Applications

Also provided are methods of diagnosing disease states based on observed levels and/or activity of the subject polypeptide(s) and/or the level of a subject polynucleotide in a biological sample of interest. Samples, as used herein, include biological fluids such as blood, cerebrospinal fluid, tears, saliva, lymph, dialysis fluid, lavage fluid, semen and the like; cells; organ or tissue culture derived fluids; tissue biopsy samples; tumor biopsy samples; stool samples; and fluids extracted from physiological tissues. Also included in the term are derivatives and fractions of such fluids. The cells may be dissociated, in the case of solid tissues, or tissue sections may be analyzed. Alternatively a lysate of the cells may be prepared.

Detection methods of the invention may be qualitative or quantitative. Thus, as used herein, the terms “detection,” “determination,” and the like, refer to both qualitative and quantitative determinations, and include “measuring.”

Detection methods of the present invention include methods for detecting polypeptide polypeptide in a biological sample, methods for detecting polynucleotide mRNA in a biological sample, and methods for detecting polypeptide enzymatic activity in a biological sample.

Detection Kits

The detection methods can be provided as part of a kit. Thus, the invention further provides kits for detecting the presence and/or a level of a subject polypeptide or subject polynucleotide in a biological sample. Procedures using these kits can be performed by clinical laboratories, experimental laboratories, medical practitioners, or private individuals. The kits of the invention for detecting a subject polypeptide comprise a moiety that specifically binds a subject polypeptide, including, but not limited to, a polypeptide-specific antibody. The kits of the invention for detecting a polynucleotide polynucleotide comprise a moiety that specifically hybridizes to a subject polynucleotide.

In some embodiments, a kit of the invention for detecting a subject polynucleotide, such as an mRNA encoding a subject polypeptide, comprises a pair of nucleic acids that function as “forward” and “reverse” primers that specifically amplify a cDNA copy of a subject polypeptide-encoding mRNA. The “forward” and “reverse” primers are provided in the kit as a pair of isolated nucleic acid molecules, each from about 10 to 200 nucleotides in length, the first nucleic acid molecule of the pair comprising a sequence of at least 10 contiguous nucleotides having 100% sequence identity to a nucleic acid sequence shown in the Sequence listing, and the second nucleic acid molecule of the pair comprising a sequence of at least 10 contiguous nucleotides having 100% sequence identity to the reverse complement of a nucleic acid sequence shown in the Sequence listing, wherein the sequence of the second nucleic acid molecule is located 3′ of the nucleic acid sequence of the first nucleic acid molecule. The primer nucleic acids are prepared using any known method, e.g., automated synthesis, and the like.

The invention provides a kit comprising a pair of nucleic acids as described above. The nucleic acids are present in a suitable storage medium, e.g., buffered solution, typically in a suitable container. The kit includes the pair of nucleic acids, and may further include a buffer; reagents for polymerase chain reaction (e.g., deoxynucleotide triphosphates (dATP, dTTP, dCTP, and dGTP), a thermostable DNA polymerase, a buffer suitable for polymerase chain reaction, a solution containing Mg²⁺ ions (e.g., MgCl₂), and other components well known to those skilled in the art for carrying out a polymerase chain reaction). The kit may further include instructions for use of the kit, which instructions may be provided in a variety of forms, e.g., as printed information, on a compact disc, and the like. The kit may further include reagents necessary for extraction of DNA from a biological sample (e.g., biopsy sample, blood, and the like) from an individual, and reagents for generating a cDNA copy of an mRNA.

The kits are useful in diagnostic applications, as described in more detail below. The pair of isolated nucleic acid molecules serve as primers in an amplification reaction (e.g., a polymerase chain reaction).

In some embodiments, the first and/or the second nucleic acid molecules comprises a detectable label. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³² P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The kit may optionally provide additional components that are useful in the procedure, including, but not limited to, buffers, developing reagents, labels, reacting surfaces, means for detections, control samples, standards, instructions, and interpretive information.

Where the kit provides for detection of a subject polypeptide, the kit includes one or more antibodies specific for the subject polypeptide. In some embodiments, the antibody specific for the subject polypeptide is detectably labeled. In other embodiments, the antibody specific for the subject polypeptide is not labeled; instead, a second, detectably-labeled antibody is provided that binds to the antibody specific for a subject polypeptide (the “first” antibody). The kit may further include blocking reagents, buffers, and reagents for developing and/or detecting the detectable marker.

The kit may further include instructions for use, controls, and interpretive information.

Where the kit provides for detecting enzymatic activity of a subject polypeptide, the kit includes a substrate that provides for a detectable product when acted upon by a subject polypeptide. The kit may further include reagents necessary for detectable marker development and detection. The kit may further include instructions for use, controls, and interpretive information.

Methods of Detecting a Subject Polypeptide in a Biological Sample

The present invention further provides methods for detecting the presence and/or measuring a level of a polypeptide in a biological sample, using an antibody specific for a subject polypeptide. The methods generally comprise:

a) contacting the sample with an antibody specific for a subject polypeptide; and

b) detecting binding between the antibody and molecules of the sample.

Detection of specific binding of the antibody, when compared to a suitable control, is an indication that a subject polypeptide is present in the sample. Suitable controls include a sample known not to contain a subject polypeptide; and a sample contacted with an antibody not specific for the subject polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, â-galactosidase, and the like); fluorescent labels (e.g., fluorescein isothiocyanate, rhodamine, phycoerythrin, and the like); fluorescence emitting metals, e.g., 152Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like.

The antibody may be attached (coupled) to an insoluble support, such as a polystyrene plate or a bead. Indirect labels include second antibodies specific for the specific antibodies, wherein the second antibody is labeled as described above; and members of specific binding pairs, e.g., biotin-avidin, and the like. The biological sample may be brought into contact with an immobilized on a solid support or carrier, such as nitrocellulose, that is capable of immobilizing cells, cell particles, or soluble proteins. The support may then be washed with suitable buffers, followed by contacting with a detectably-labeled specific antibody. Detection methods are known in the art and will be chosen as appropriate to the signal emitted by the detectable label. Detection is generally accomplished in comparison to suitable controls, and to appropriate standards.

Methods of Detecting Enzymatic Activity of a Subject Polypeptide in a Biological Sample

The present invention further provides methods for detecting the presence and/or levels of enzymatic activity of a subject polypeptide in a biological sample. The methods generally involve:

a) contacting the sample with a substrate that yields a detectable product upon being acted upon by a subject polypeptide; and

b) detecting a product of the enzymatic reaction. Methods of detecting a biological activity of a subject polypeptide in a biological sample

The present invention further provides methods for detecting the presence and/or levels of a biological activity of a subject polypeptide in a biological sample. The methods generally involve:

a) contacting the sample with a substrate that is acted on by the subject protein; and

b) determining the effect, if any, of the subject protein on the substrate.

Suitable substrates include, but are not limited to, a cell that provides for a readout when contacted with the subject protein (e.g., cell proliferation, secretion of a factor, cell death, cell migration, and the like); a factor that binds to a subject protein; and the like.

Methods of Detecting a Polynucleotide mRNA in a Biological Sample

The present invention further provides methods for detecting the presence of a subject polynucleotide mRNA in a biological sample. The methods can be used, for example, to assess whether a test compound affects subject gene expression, directly or indirectly.

The methods generally comprise:

a) contacting the sample with a polynucleotide of the invention under conditions which allow hybridization; and

b) detecting hybridization, if any.

Detection of hybridization, when compared to a suitable control, is an indication of the presence in the sample of a subject polynucleotide. Appropriate controls include, for example, a sample which is known not to contain subject polynucleotide mRNA, and use of a labelled polynucleotide of the same “sense” as a subject polynucleotide mRNA.

Conditions which allow hybridization are known in the art, and have been described in more detail above. Detection can be accomplished by any known method, including, but not limited to, in situ hybridization, PCR, RT-PCR, and “Northern” or RNA blotting, or combinations of such techniques, using a suitably labeled subject polynucleotide. A variety of labels and labeling methods for polynucleotides are known in the art and can be used in the assay methods of the invention. Specific hybridization can be determined by comparison to appropriate controls.

In some embodiments, the methods involve generating a cDNA copy of an mRNA molecule in a biological sample, and amplifying the cDNA using a pair of isolated nucleic acid molecules that serve as forward and reverse primers in an amplification reaction (e.g., a polymerase chain reaction). Each of the nucleic acid molecules in the pair of nuclei acid molecules is from about 10 to 200 nucleotides in length, the first nucleic acid molecule of the pair comprising a sequence of at least 10 contiguous nucleotides having 100% sequence identity to a nucleic acid sequence shown in the Sequence listing, and the second nucleic acid molecule of the pair comprising a sequence of at least 10 contiguous nucleotides having 100% sequence identity to the reverse complement of a nucleic acid sequence set forth in the Sequence listing, wherein the sequence of the second nucleic acid molecule is located 3′ of the nucleic acid sequence of the first nucleic acid molecule. The primer nucleic acids are prepared using any known method, e.g., automated synthesis, and the like. The primer pairs are chosen such that they specifically amplify a cDNA copy of an mRNA encoding a subject polypeptide.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp.14.2B14.33; McPherson et al. PCR Basics: From Background to Bench (2000) Springer Verlag; Dieffenbach and Dveksler PCR Primer: A Laboratory Manual (1995) Cold Spring Harbor Laboratory Press.

A detectable label may be included in the amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵ S, ³H; etc.

The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

A number of methods are available for determining the expression level of a gene or protein in a particular sample. Diagnosis may be performed by a number of methods to determine the absence or presence or altered amounts of normal or abnormal subject polypeptide in a patient sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc.

Alternatively, one may focus on the expression of the subject genes. Biochemical studies may be performed to determine whether a sequence polymorphism in a coding region or control regions is associated with disease. Disease associated polymorphisms may include deletion or truncation of the gene, mutations that alter expression level, that affect the activity of the protein, etc.

Changes in the promoter or enhancer sequence that may affect expression levels of the subject genes can be compared to expression levels of the normal allele by various methods known in the art. Methods for determining promoter or enhancer strength include quantitation of the expressed natural protein; insertion of the variant control element into a vector with a reporter gene such as α-galactosidase, luciferase, chloramphenicol acetyltransferase, etc. that provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express the gene may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. (1985), Science 239:487, and a review of techniques may be found in Sambrook, et al. Molecular Cloning: A Laboratory Manual, CSH Press 1989, pp.14.2-14.33. Alternatively, various methods are known in the art that utilize oligonucleotide ligation as a means of detecting polymorphisms, for examples see Riley et al. (1990), Nucl. Acids Res. 18:2887-2890; and Delahunty et al. (1996), Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. The nucleic acid may be sequenced by dideoxy or other methods, and the sequence of bases compared to a wild-type sequence.

Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in the gene may be based on the functional or antigenic characteristics of the protein. Protein truncation assays are useful in detecting deletions that may affect the biological activity of the protein. Various immunoassays designed to detect polymorphisms in proteins may be used in screening. Where many diverse genetic mutations lead to a particular disease phenotype, functional protein assays have proven to be effective screening tools. The activity of the encoded protein may be determined by comparison with the wild-type protein.

Diagnostic methods of the subject invention in which the level of expression is of interest will typically involve comparison of the nucleic acid abundance of a sample of interest with that of a control value to determine any relative differences, where the difference may be measured qualitatively and/or quantitatively, which differences are then related to the presence or absence of an abnormal expression pattern. A variety of different methods for determining the nucleic acid abundance in a sample are known to those of skill in the art, where particular methods of interest include those described in: Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

Methods of Identifying Biological Molecules that Interact with a Subject Polypeptide

Formation of a binding complex between a subject polypeptide and an interacting polypeptide or other macromolecule (e.g., DNA, RNA, lipids, polysaccharides, and the like) can be detected using any known method. Suitable methods include: a yeast two-hybrid method; a mammalian cell two-hybrid method; a FRET assay; a BRET assay; a fluorescence quenching assay; a fluorescence anisotropy assay; an immunological assay; and an assay involving binding of a detectably labeled protein to an immobilized protein.

Fluorescence anisotropy assays are amply described in the literature. See, e.g., Jameson and Sawyer (1995) Methods Enzymol. 246:283-300. The yeast two-hybrid assay system has been described in the literature. See, e.g., Zhu and Kahn (1997) Proc. Natl. Acad. Sci. U.S.A. 94:13063-13068; Fields and Song (1989) Nature 340:245-246; and U.S. Pat. No. 5,283,173; Chien et al. (1991) Proc. Natl. Acad. Sci. U.S.A. 88:9578-9581.

Immunological assays, and assays involving binding of a detectably labeled protein to an immobilized protein can be arranged in a variety of ways.

Immunoprecipitation assays can be designed, wherein the subject protein/interacting polypeptide complex is detected by precipitating the complex with antibody specific for the subject protein and/or the interacting polypeptide.

In other embodiments, the assay is a binding assay which detects binding of a subject protein to an immobilized protein, or which detects binding of a protein to immobilized subject protein. In some embodiments, the subject polypeptide is labeled with a detectable label, and binding to an immobilized interacting polypeptide is detected. In other embodiments, the interacting polypeptide is labeled with a detectable label, and binding to an immobilized subject polypeptide is detected. In other embodiments, the subject polypeptide is immobilized, and binding of the interacting polypeptide to the subject polypeptide is detected using an antibody specific for the interacting polypeptide, where the antibody is either directly labeled or a secondary antibody that is labeled is used. In other embodiments, the interacting polypeptide is immobilized, and binding of the subject polypeptide to the interacting polypeptide is detected using an antibody specific for the subject polypeptide, where the antibody is either directly labeled or a secondary antibody that is labeled is used.

Formation of a binding complex between a subject polypeptide and an interacting polypeptide can also be detected using fluorescence resonance energy transfer (FRET), bioluminescence resonance energy transfer (BRET), anisotropy measurements, and fluorescence quenching measurements.

FRET involves the transfer of energy from a donor fluorophore in an excited state to a nearby acceptor fluorophore. For this transfer to take place, the donor and acceptor molecules must in close proximity (e.g., less than 10 nanometers apart, usually between 10 and 100 Å apart), and the emission spectra of the donor fluorophore must overlap the excitation spectra of the acceptor fluorophore. In these embodiments, a fluorescently labeled subject protein serves as a donor and/or acceptor in combination with a second fluorescent protein or dye, e.g., a fluorescent protein as described in Matz et al., Nature Biotechnology (October 1999) 17:969-973; a green fluorescent protein (GFP), including a “humanized” GFP; a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304, the disclosures of which are herein incorporated by reference; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); other fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye, etc., chemilumescent dyes, e.g., luciferases.

BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by analyzing transfer between a bioluminescent donor protein and a fluorescent acceptor protein. Interaction between the donor and acceptor proteins can be monitored by a change in the ratio of light emitted by the bioluminescent and fluorescent proteins. In this application, the subject protein serves as donor and/or acceptor protein.

Fluorescent subject protein can be produced by generating a construct comprising a subject protein and a fluorescent fusion partner, e.g., a fluorescent protein as described in Matz et al. ((1999) Nature Biotechnology 17:969-973), a green fluorescent protein from any species or a derivative thereof; e.g., a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); a GFP from Aequoria victoria or fluorescent mutant thereof, e.g., as described in U.S. Pat. Nos. 6,066,476; 6,020,192; 5,985,577; 5,976,796; 5,968,750; 5,968,738; 5,958,713; 5,919,445; 5,874,304. Generation of such a construct, and production of a subject protein/fluorescent protein fusion protein is well within the skill level of those of ordinary skill in the art.

Fluorescent subject protein can also be generated by producing the subject protein in an auxotrophic strain of bacteria which requires addition of one or more amino acids in the medium for growth. A subject protein-encoding construct that provides for expression in bacterial cells is introduced into the auxotrophic strain, and the bacteria are cultured in the presence of a fluorescent amino acid, which is incorporated into the subject protein produced by the bacterium. The subject protein is then purified from the bacterial culture using standard methods for protein purification.

Where the interacting protein is at least a second subject protein, the effect of the test agent on binding can be determined by determining the effect on multimerization of the subject protein. As used herein, the term “multimerization” refers to formation of dimers, trimers, tetramers, and higher multimers of the subject protein. Whether a subject protein forms a complex with one or more additional subject protein molecules can be determined using any known assay, including assays as described above for interacting proteins. Formation of multimers can also be detected using non-denaturing gel electrophoresis, where multimerized subject protein migrates more slowly than monomeric subject protein. Formation of multimers can also be detected using fluorescence quenching techniques.

Formation of multimers can also be detected by analytical ultracentrifugation, for example through glycerol or sucrose gradients, and subsequent visualization of a subject protein in gradient fractions by Western blotting or staining of SDS-polyacrylamide gels.

Multimers are expected to sediment at defined positions in such gradients. Formation of multimers can also be detected using analytical gel filtration, e.g. in HPLC or FPLC systems, e.g. on columns such as Superdex 200 (Pharmacia Amersham Inc.). Multimers run at defined positions on these columns, and fractions can be analyzed as above. The columns are highly reproducible, allowing one to relate the number and position of peaks directly to the multimerization status of the protein.

Screening Assays

The present invention provides screening methods for identifying agents which modulate a biological activity of a subject polypeptide, methods for identifying agents which modulate a level of a subject polypeptide in a cell; and methods for identifying agents which modulate a level of a subject polynucleotide mRNA in a cell. In some embodiments, the assay is a cell-free assay. In other embodiments, the assay is a cellbased assay.

As used herein, the term “modulate” encompasses “increase” and “decrease.” In some embodiments, of particular interest are agents which inhibit a biological activity of a subject polypeptide, and/or which reduce a level of a subject polypeptide in a cell, and/or which reduce a level of a subject mRNA in a cell and/or which reduce release of a subject polypeptide from a eukaryotic cell. In other embodiments, agents of interest are those that increase polypeptide activity.

The terms “candidate agent,” “agent”, “substance” and “compound” are used interchangeably herein. Candidate agents encompass numerous chemical classes, typically synthetic, semi-synthetic, or naturally occurring inorganic or organic molecules. Candidate agents may be small organic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups.

Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries.

Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal.

Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc.

For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening. Typically between 0.1 and 1 hour will be sufficient.

Methods for Identifying Agents that Modulate a Biological Activity of a Subject Polypeptide

The present invention provides methods of identifying agents that modulate a biological activity of a polypeptide of the invention. The term “modulate” encompasses an increase or a decrease in the measured activity when compared to a suitable control.

The method generally comprises:

a) contacting a test agent with a sample containing a subject polypeptide; and

b) assaying a biological activity of the subject polypeptide in the presence of the test agent. An increase or a decrease in the assayed biological activity in comparison to the activity in a suitable control (e.g., a sample comprising a subject polypeptide in the absence of the substance being tested) is an indication that the substance modulates a biological activity of the subject polypeptide.

In some embodiments, the assays are cell-free assays. In other embodiments, the assays are cell-based assays. Cell-based assays generally involve contacting a cell that produces a subject protein with a test agent; and determining the effect, if any, of the test agent on the activity of the subject protein.

In some embodiments, the biological activity is an effect on a cell in in vitro culture. In these embodiments, the methods involve contacting a cell in in vitro culture with a subject protein and a test agent, and determining the effect, if any, of the test protein on the effect of the subject protein on the cell. Effects include, but are not limited to, cell proliferation, secretion of a factor from the cell, cell death, cell migration, and the like.

In some embodiments, the biological activity is an effect on a cell in vivo. In these embodiments, the methods involve contacting a cell in vivo in a non-human animal with a subject protein and a test agent, and determining the effect, if any, of the agent on the activity of the subject protein on the animal. Effects include, but are not limited to, immune response, appetite, blood pressure, and the like.

An “agent that modulates a biological activity of a subject polypeptide”, as used herein, describes any molecule, e.g. synthetic or natural organic or inorganic compound, protein or pharmaceutical, with the capability of altering a biological activity of a subject polypeptide, as described herein. Generally a plurality of assay mixtures is run in parallel with different agent concentrations to obtain a differential response to the various concentrations. Typically, one of these concentrations serves as a negative control, i.e. at zero concentration or below the level of detection. The biological activity can be measured using any assay known in the art.

An agent which modulates a biological activity of a subject polypeptide increases or decreases the activity at least about 10%, at least about 15%, at least about 20%, at least about 25%, more preferably at least about 50%, more preferably at least about 100%, or 2-fold, more preferably at least about 5-fold, more preferably at least about 10-fold or more when compared to a suitable control.

Agents that increase or decrease a biological activity of a subject polypeptide to the desired extent may be selected for further study, and assessed for cellular availability, cytotoxicity, biocompatibility, etc.

In some embodiments, of particular interest are agents that decrease a biological activity of a subject polypeptide. Maximal inhibition of the activity is not always necessary, or even desired, in every instance to achieve a therapeutic effect. Agents which decrease a biological activity of a subject polypeptide may find use in treating disorders associated with the biological activity of the polypeptide.

Of particular interest in some embodiments are agents that increase a biological activity of a subject polypeptide. Agents which increase a biological activity of a subject polypeptide may find use in treating disorders associated with a deficiency in the biological activity.

Cell-Based Methods

Cell-based methods include methods of detecting an agent that modulates a level of a subject polynucleotide mRNA and/or subject polypeptide.

A candidate agent is assessed for any cytotoxic activity it may exhibit toward the cell used in the assay, using well-known assays, such as trypan blue dye exclusion, an MTT ([3-(4,5-dimethylthiazol-2-yl)-2,5-diphenyl-2H-tetrazolium bromide]) assay, and the like. Agents that do not exhibit cytotoxic activity are considered candidate agents.

The cells used in the assay are usually mammalian cells, including, but not limited to, rodent cells and human cells. The cells may be primary cell cultures or may be immortalized cell lines.

Methods of Detecting Agents that Modulate a Level of Subject mRNA and/or Subject Polypeptide

A wide variety of cell-based assays may be used for identifying agents which modulate levels of a subject mRNA in a eukaryotic cell, using, for example, a mammalian cell transformed with a construct comprising a subject cDNA such that the cDNA is overexpressed, or, alternatively, a construct comprising a promoter endogenously associated with a subject polynucleotide (or its genomic counterpart) operably linked to a reporter gene.

Accordingly, the present invention provides a method for identifying an agent, particularly a biologically active agent, that modulates a level of expression of a subject nucleic acid in a cell, the method comprising: combining a candidate agent to be tested with a cell comprising a nucleic acid which encodes a subject polypeptide; and determining the effect of said agent on expression of the subject polynucleotide.

“Modulation” of expression levels includes increasing the level and decreasing the level of subject mRNA and/or subject polypeptide encoded by the subject polynucleotide when compared to a control lacking the agent being tested. An increase or decrease of about 1.25-fold, usually at least about 1.5-fold, usually at least about 2-fold, usually at least about 5-fold, usually at least about 10-fold or more, in the level (i.e., an amount) of subject mRNA and/or polypeptide following contacting the cell with a candidate agent being tested, compared to a control to which no agent is added, is an indication that the agent modulates subject polynucleotide expression.

Subject mRNA and/or polypeptide whose levels are being measured can be encoded by an endogenous polynucleotide corresponding to a subject nucleic acid, or the polynucleotide can be one that is comprised within a recombinant vector and introduced into the cell, i.e., the subject mRNA and/or polypeptide can be encoded by an exogenous polynucleotide. For example, a recombinant vector may comprise an isolated polynucleotide transcriptional regulatory sequence, such as a promoter sequence, operably linked to a reporter gene (e.g., α-galactosidase, CAT, luciferase, or other gene that can be easily assayed for expression).

In these embodiments, the method for identifying an agent that modulates a level of expression of a subject polynucleotide in a cell, comprises: combining a candidate agent to be tested with a cell comprising a nucleic acid which comprises a subject gene transcriptional regulatory element operably linked to a reporter gene; and determining the effect of said agent on reporter gene expression. A recombinant vector may comprise an isolated transcriptional regulatory sequence which is associated in nature with a subject nucleic acid, such as a promoter sequence, operably linked to sequences coding for a subject polypeptide; or the transcriptional control sequences can be operably linked to coding sequences for a subject polypeptide fusion protein comprising a subject polypeptide fused to a polypeptide which facilitates detection. In these embodiments, the method comprises combining a candidate agent to be tested with a cell comprising a nucleic acid which comprises a subject polynucleotide gene transcriptional regulatory element operably linked to a subject polypeptide-coding sequence; and determining the effect of said agent on subject polynucleotide expression, which determination can be carried out by measuring an amount of subject mRNA, subject polypeptide, or subject fusion polypeptide produced by the cell.

Cell-based assays generally comprise the steps of contacting the cell with an agent to be tested, forming a test sample, and, after a suitable time, assessing the effect of the agent on expression of a subject polynucleotide. A control sample comprises the same cell without the candidate agent added. Expression levels are measured in both the test sample and the control sample. A comparison is made between subject polynucleotide expression level in the test sample and the control sample. Expression can be assessed using conventional assays. For example, when a mammalian cell line is transformed with a construct that results in expression of the subject polynucleotide, subject mRNA levels can be detected and measured, as described above, or subject polypeptide levels can be detected and measured, as described above. A suitable period of time for contacting the agent with the cell can be determined empirically, and is generally a time sufficient to allow entry of the agent into the cell and to allow the agent to have a measurable effect on subject mRNA and/or polypeptide levels. Generally, a suitable time is between 10 minutes and 24 hours, more typically about 1-8 hours.

Methods of measuring subject mRNA levels are known in the art, several of which have been described above, and any of these methods can be used in the methods of the present invention to identify an agent which modulates subject mRNA level in a cell, including, but not limited to, a PCR, such as a PCR employing detectably labeled oligonucleotide primers, and any of a variety of hybridization assays. Similarly, subject polypeptide levels can be measured using any standard method, several of which have been described herein, including, but not limited to, an immunoassay such as ELISA, for example an ELISA employing a detectably labeled antibody specific for a subject polypeptide.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used.

The screening methods may be designed a number of different ways, where a variety of assay configurations and protocols may be employed, as are known in the art.

For example, one of the components may be bound to a solid support, and the remaining components contacted with the support bound component. The above components of the method may be combined at substantially the same time or at different times.

Incubations are performed at any suitable temperature, typically between 4 and 40° C.

Incubation periods are selected for optimum activity, but may also be optimized to facilitate rapid high-throughput screening. Typically between 0.1 and 1 hours will be sufficient. Following the contact and incubation steps, the subject methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound complexes will then be detected.

A variety of different candidate agents may be screened by the above methods.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents

The invention further provides agents identified using a screening assay of the invention, and compositions comprising the agents, including pharmaceutical compositions. In addition, the subject proteins themselves are “agents,” and are useful in a variety of therapeutic methods. The subject compositions can be formulated using well-known reagents and methods. In some embodiments, compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Nucleic Acid and Polypeptide Therapeutic Compositions

The nucleic acid compositions and polypeptide compositions of the subject invention also find use as therapeutic agents in situations where one wishes to enhance an activity of a subject polypeptide in a host, particularly the activity of the subject polypeptides, or to provide the activity at a particular anatomical site.

In some embodiments, a subject polypeptide is provided in a pharmaceutical composition with a pharmaceutically acceptable excipient. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The subject genes, gene fragments, or the encoded proteins or protein fragments are useful in therapy to treat disorders associated with an activity of a subject polypeptide. Expression vectors may be used to introduce the gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

In yet other embodiments of the invention, the active agent is an agent that modulates, and generally decreases or down regulates, the expression of the gene encoding the target protein in the host. For example, antisense molecules can be used to down-regulate expression of the subject genes in cells. The anti-sense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model.

A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which modifications alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The â-anomer of deoxyribose may be used, where the base is inverted with respect to the natural á-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression.

Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In some embodiments, the active agent is an interfering RNA (RNAi). RNAi includes double-stranded RNA interference (dsRNAi). Use of RNAi to reduce a level of a particular mRNA and/or protein is based on the interfering properties of doublestranded RNA derived from the coding regions of gene. In one example of this method, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject (such as in their food or by soaking in the buffer containing the RNA). See, e.g., WO99/32619. In another embodiment, dsRNA derived from a subject gene is generated in vivo by simultaneous expression of both sense and antisense RNA from appropriately positioned promoters operably linked to subject coding sequences in both sense and antisense orientations.

In some embodiments, an active agent is a peptide. Suitable peptides include peptides of from about 3 amino acids to about 50, from about 5 to about 30, or from about 10 to about 25 amino acids in length. In some embodiments, a peptide exhibits one or more of the following activities: inhibits binding of a subject polypeptide to a an interacting protein; inhibits subject polypeptide binding to a second polypeptide molecule; inhibits a signal transduction activity of a subject polypeptide; inhibits an enzymatic activity of a subject polypeptide; inhibits a DNA binding activity of a subject polypeptide. In some embodiments, a peptide has a sequence of from about 3 amino acids to about 50, from about 5 to about 30, or from about 10 to about 25 amino acids of corresponding naturally-occurring protein.

Peptides can include naturally-occurring and non-naturally occurring amino acids. Peptides may comprise D-amino acids, a combination of D- and L-amino acids, and various “designer” amino acids (e.g., β-methyl amino acids, Ca-methyl amino acids, and Na-methyl amino acids, etc.) to convey special properties to peptides. Additionally, peptide may be a cyclic peptide. Peptides may include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methylphenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; â-carboline (D and L); HIC (histidine isoquinoline carboxylic acid); and HIC (histidine cyclic urea). Amino acid analogs and peptidomimetics may be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; β-helix inducing analogs; β-turn inducing analogs; Gly-Ala turn analog; amide bond isostere; tretrazol; and the like.

A peptide may be a depsipeptide, which may be a linear or a cyclic depsipeptide.

Kuisle et al. (1999) Tet. Letters 40:1203-1206. “Depsipeptides” are compounds containing a sequence of at least two alpha-amino acids and at least one alpha-hydroxy carboxylic acid, which are bound through at least one normal peptide link and ester links, derived from the hydroxy carboxylic acids, where “linear depsipeptides” may comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. “Cyclic depsipeptides” are peptides containing at least one ring formed only through peptide or ester links, derived from hydroxylcarboxylic acids.

Peptides may be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine.

Internal cyclization as opposed to polymerization can be enhanced by use of very dilute solutions. Methods for making cyclic peptides are well known in the art

The term “bicyclic” refers to a peptide in which there exists two ring closures.

The ring closures are formed by covalent linkages between amino acids in the peptide. A covalent linkage between two nonadjacent amino acids constitutes a ring closure, as does a second covalent linkage between a pair of adjacent amino acids which are already linked by a covalent peptide linkage. The covalent linkages forming the ring closures may be amide linkages, i.e., the linkage formed between a free amino on one amino acid and a free carboxyl of a second amino acid, or linkages formed between the side chains or “R” groups of amino acids in the peptides. Thus, bicyclic peptides may be “true” bicyclic peptides, i.e., peptides cyclized by the formation of a peptide bond between the N-terminus and the C-terminus of the peptide, or they may be “depsi-bicyclic” peptides, i.e., peptides in which the terminal amino acids are covalently linked through their side chain moieties.

A desamino or descarboxy residue can be incorporated at the terminii of the peptide, so that there is no terminal amino or carboxyl group, to decrease susceptibility to proteases or to restrict the conformation of the peptide. C-terminal functional groups include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S., Bioconjugate Chem., 6:150-165 (1995); Monfardini, C, et al., Bioconjugate Chem., 6:62-69 (1995); U.S. Pat. Nos. 4,640,835; 4,496,689; 4,301,144; 4,670,417; 4,791,192; 4,179,337 or WO 95/34326.

Another suitable agent for reducing an activity of a subject polypeptide is a peptide aptamer. Peptide aptamers are peptides or small polypeptides that act as dominant inhibitors of protein function. Peptide aptamers specifically bind to target proteins, blocking their function ability. Kolonin and Finley, PNAS (1998) 95:14266-14271. Due to the highly selective nature of peptide aptamers, they may be used not only to target a specific protein, but also to target specific functions of a given protein (e.g. a signaling function). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Peptide aptamers that bind with high affinity and specificity to a target protein may be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

Intracellularly expressed antibodies, or intrabodies, are single-chain antibody molecules designed to specifically bind and inactivate target molecules inside cells.

Intrabodies have been used in cell assays and in whole organisms. Chen et al., Hum. Gen. Ther. (1994) 5:595-601; Hassanzadeh et al., Febs Lett. (1998) 16(1, 2):75-80 and 81-86. Inducible expression vectors can be constructed with intrabodies that react specifically with subject protein. These vectors can be introduced into model organisms and studied in the same manner as described above for aptamers.

Therapeutic Methods

The instant invention provides various therapeutic methods. In some embodiments, methods of modulating, including increasing and inhibiting, enzymatic activity of the subject proteins are provided. In other embodiments, methods of modulating a signal transduction activity of a subject protein is provided. In other embodiments, methods of modulating interaction of a subject protein with another, interacting protein are provided.

The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

As used herein, the term “agent” refers to a substance that modulates a level or activity of active subject polypeptide or level of mRNA encoding a subject protein.

Where the agent is a substance that modulates a level of activity of a subject polypeptide, agents include antibodies specific for the subject polypeptide, peptide aptamers, small molecules, agents that bind a ligand-binding site in a subject polypeptide, and the like. Where the agent modulates a level of mRNA encoding a subject protein, agents include antisense, ribozymes, and RNAi. The term “agent” also refers to substances that modulate a condition or disorder associated with a subject polypeptide. Such agents include subject polypeptides themselves, subject polynucleotides, and the like.

In some embodiments, an agent is one identified by a screening assay of the invention. “Modulating a level of active subject polypeptide” includes increasing or decreasing activity of a subject polypeptide; increasing or decreasing a level of active polypeptide protein; and increasing or decreasing a level of mRNA encoding active subject polypeptide. In some embodiments, an agent is a subject polypeptide, where the subject polypeptide itself is administered to an individual. In some embodiments, an agent is an antibody specific for a subject polypeptide.

Formulations, Dosages, and Routes of Administration

As mentioned above, an effective amount of the active agent (e.g., small molecule, antibody specific for a subject polypeptide, or a subject polypeptide) is administered to the host, where “effective amount” means a dosage sufficient to produce a desired result. In some embodiments, the desired result is at least a reduction in enzymatic activity of a subject polypeptide as compared to a control. In other embodiments, the desired result is an increase in the level of enzymatically active subject polypeptide (in the individual, or in a localized anatomical site in the individual), as compared to a control.

Typically, the compositions of the instant invention will contain from less than 1% to about 95% of the active ingredient, preferably about 10% to about 50%.

Generally, between about 100 mg and 500 mg will be administered to a child and between about 500 mg and 5 grams will be administered to an adult. Administration is generally by injection and often by injection to a localized area. The frequency of administration will be determined by the care given based on patient responsiveness.

Other effective dosages can be readily determined by one of ordinary skill in the art through routine trials establishing dose response curves.

In order to calculate the amount of subject polypeptide, those skilled in the art could use readily available information with respect to the amount of polypeptide necessary to have a the desired effect. The amount of an agent necessary to increase a level of active subject polypeptide can be calculated from in vitro experimentation. The amount of agent will, of course, vary depending upon the particular agent used.

In the subject methods, the active agent(s) may be administered to the host using any convenient means capable of resulting in the desired inhibition of activity of the polypeptide. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds.

The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the polypeptide adequate to achieve the desired state in the subject being treated.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as inflammation and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The agents of the present invention can be used by themselves, with each other, or in combination with pharmaceutically acceptable excipient materials as described above.

Kits with unit doses of the active agent, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

In some embodiments, a subject protein is involved in control cell proliferation, and a subject agent reduces cell proliferation, e.g., unwanted cellular proliferation.

Such agents are useful in cancer treatments. Whether a particular agent and/or therapeutic regimen of the invention is effective in reducing unwanted cellular proliferation, e.g., in the context of treating cancer, can be determined using standard methods. For example, the number of cancer cells in a biological sample (e.g., blood, a biopsy sample, and the like), can be determined. The tumor mass can be determined using standard radiological methods.

Tumors which may be treated using the methods of the instant invention include carcinomas, e.g. colon, prostate, breast, melanoma, ductal, endometrial, stomach, pancreactic, mesothelioma, dysplastic oral mucosa, invasive oral cancer, non-small cell lung carcinoma, transitional and squamous cell urinary carcinoma, etc.; neurological malignancies, e.g. neuroblastoma, glioblastoma, astrocytoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

In other embodiments, e.g., where the subject polypeptide is involved in modulating inflammation, the invention provides agents for treating inflammation.

Disease states which are treatable using formulations of the invention include various types of arthritis, various chronic inflammatory conditions of the skin, insulin-dependent diabetes, and adult respiratory distress syndrome. After reading the present disclosure, those skilled in the art will recognize other disease states and/or symptoms which might be treated and/or mitigated by the administration of formulations of the present invention.

Where a subject polypeptide is involved in modulating cell death, an agent of the invention is useful for treating disorders relating to cell death (e.g., DNA damage, cell death, apoptosis). Cell death-related indications which can be treated using the methods of the invention for reducing cell death in a eukaryotic cell, include, but are not limited to, cell death associated with Alzheimer's disease, Parkinson's disease, rheumatoid arthritis, septic shock, sepsis, stroke, central nervous system inflammation, osteoporosis, ischemia, reperfusion injury, cell death associated with cardiovascular disease, polycystic kidney disease, cell death of endothelial cells in cardiovascular disease, degenerative liver disease, multiple sclerosis, amyotropic lateral sclerosis, cerebellar degeneration, ischemic injury, cerebral infarction, myocardial infarction, acquired immunodeficiency syndrome (AIDS), myelodysplastic syndromes, aplastic anemia, male pattern baldness, and head injury damage. Also included are conditions in which DNA damage to a cell is induced by, e.g., irradiation, radiomimetic drugs, and the like. Also included are any hypoxic or anoxic conditions, e.g., conditions relating to or resulting from ischemia, myocardial infarction, cerebral infarction, stroke, bypass heart surgery, organ transplantation, neuronal damage, and the like.

DNA damage can be detected using any known method, including, but not limited to, a Comet assay (commercially available from Trevigen, Inc.), which is based on alkaline lysis of labile DNA at sites of damage; and immunological assays using antibodies specific for aberrant DNA structures, e.g., 8-OHdG.

Cell death can be measured using any known method, and is generally measured using any of a variety of known methods for measuring cell viability. Such assays are generally based on entry into the cell of a detectable compound (or a compound that becomes detectable upon interacting with, or being acted on by, an intracellular component) that would normally be excluded from a normal, living cell by its intact cell membrane. Such compounds include substrates for intracellular enzymes, including, but not limited to, a fluorescent substrate for esterase; dyes that are excluded from living cell, including, but not limited to, trypan blue; and DNA-binding compounds, including, but not limited to, an ethidium compound such as ethidium bromide and ethidium homodimer, and propidium iodide.

Apoptosis can be assayed using any known method. Assays can be conducted on cell populations or an individual cell, and include morphological assays and biochemical assays. A non-limiting example of a method of determining the level of apoptosis in a cell population is TUNEL (TdT-mediated dUTP nick-end labeling) labeling of the 3′-OH free end of DNA fragments produced during apoptosis (Gavrieli et al. (1992) J. Cell Biol. 119:493). The TUNEL method consists of catalytically adding a nucleotide, which has been conjugated to a chromogen system or a to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus). Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both.

Other methods of testing for apoptosis are known in the art and can be used, including, e.g., the method disclosed in U.S. Pat. No. 6,048,703.

In some embodiments, where the subject protein affects an immune response, the subject protein is useful in methods of modulating an immune response, including, but not limited to, increasing a Th1 immune response, suppressing a Th2 immune response, increasing antibody production in response to an antigen, increasing an antigen-specific cytotoxic T lymphocyte response, reducing inflammation, inhibiting anaphylaxis, increasing antigen presentation by an antigen-presenting cell, and the like.

In some embodiments, where the subject protein modulates appetite, the subject protein is useful in methods of modulating appetite, e.g., decreasing appetite, and thus in methods of controlling weight.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The sequence data are presented in the sequence listing.

### Example 1

1) RT-PCR

<Reagents>

Ethachinmate (Nippon Gene)

DNase I (STRATAGENE)

ThermoScript RT-PCR System (Invitrogen)

HotStarTaq DNA Polymerase (QIAGEN)

Chloroform (special grade reagent) (Wako Pure Chemical Industries, Ltd.)

TE Saturated Phenol (for gene-engineering research) (Wako Pure Chemical Industries, Ltd.)

Ethanol (special grade reagent) (Wako Pure Chemical Industries, Ltd.)

Agarose-1 (Wako Pure Chemical Industries, Ltd.)

Gene Amp PCR System 9700 (Applied Biosystems)

OneSTEP Marker 4 (fX174/HaeIII digest) (Nippon Gene)

<Method>

RNAs (24 kinds) extracted from brains and major peripheral organs of C57BL/6J(B6) mice were prepared. They are listed in Table 1.

TABLE 1

Organs used for RT-PCP analysis
Organ
aorate and vein
thymus
cecum
epididymis
testis
tongue
kidney
skin
lung
heart
liver
stomach
pancreas
small intestine
cerebellum
hippocampus
cortex
olfactory bulb
hypothalamus
c. qradrigemi
pit 1
diencepharose
corpus striatum
m. oblongatu

Next, plasmids and primers for each clone to be used for this research were prepared from RIKEN Clone. The primers were prepared to have a Tm of 60° C., to have a product size of approximately 300 bp, and as much as possible, to have such a state in which an intron is sandwiched therein. Clone IDs and primer sequences are shown in Table 2.

TABLE 2

RT-PCR primers

| | | | | |
|---|---|---|---|---|
| No | Clone ID | Product Size | Primer (Left) | Primer (Right) |

| | | | | |
|---|---|---|---|---|
| 1 | A830010B16 | 174 | AACTCCTGGTTCCCACACAG | ATCCCCCAACACACACAAAT |
| 4 | C230071E12 | 207 | GAGACACAGGATCCCAGGAA | TTCAAAGCTAGGTCGCCACT |
| 6 | 9230111007 | 213 | GACACTCGAGGTCAACGTCA | CAGCAGCATGTGTGGTTTCT |
| 9 | 1700056N09 | 219 | TTCGACTTGAGGACGAAGGT | ACACGGGAGGTTACGACAAG |
| 11 | 9230110A19 | 197 | GCTGCTTCAGGTTCTCCTTG | CGACTGAGTGCTTCTTGTGC |
| 13 | A430045L05 | 196 | GCAAAACAGCTCCTGAGGTC | AGGTCCTTCACACAGGATGG |
| 19 | 4930563B01 | 232 | TACTTGTGGCAACGGAACTG | GCAGGCTGTCACGGTATTCT |
| 32 | A030004E11 | 231 | CTGGTGCTGCTAACTGGTGA | AGACAGCAGGGGTAGGGAAT |
| 44 | 1700007F22 | 177 | GCGGAACGGATATGAACACT | AAGAAAATGGGGTGGGATTC |
| 45 | B230352020 | 239 | CAAGTCGCCTCTCCTGCTAC | GGCAGATCCTCAGTGGTTGT |
| 50 | D630020P16 | 195 | CCTGGCTATTGCCCAGAGTA | CGGCTATCCTCGACTGAAAC |
| 52 | C630041L24 | 171 | TGAGCCTATATGTGGCAGCA | CCTGTCTCCCAAACTGGGTA |
| 64 | 2310031C01 | 301 | TCTCCCTCAGCCTCTCTCAG | CCACAGTCCAGACCATGTTG |
| 69 | 1110005I17 | 237 | TTACCCTACCGCAACAGAGG | GAGGCAAGGAAGATTTGTCG |
| 70 | 2310014H11 | 200 | TGCACCAATACCCAAGACAA | TCTCTGAACCCAGGATGCTT |
| 73 | A530065I17 | 193 | GTCCTAAGCAGGAGGGAACC | CCTGATAAGCGTGCAGTGAA |
| 74 | 1700011J22 | 206 | TCACTCCAGGTGATGCTCAG | CCTAGGACAGCTTTGCCATC |
| 89 | 9130004I05 | 155 | CTGTGTGCTCCACCTTGCTA | AGCCAATGATGTTCCTGTCC |
| 100 | B230114O10 | 225 | TTGGTACTATCGGCCTGACC | CCCCCTAAATTCGGTGTTTT |

Since it was confirmed by experiments that genome DNAs were mixed in with the extracted RNAs (5 μg), genome DNAs were removed with DNase I. 5 μg of RNAs were mixed in DEPC-treated water to have a final volume of 200 μl and 10U of DNase I was added to the mixture. Then, the obtained mixture was incubated at 37° C. for 15 minutes. After the mixture was treated with 200 μl of phenol/chloroform to deactivate and remove DNase I, it was centrifuged at 12,000 rpm for 10 minutes at room temperature. The supernatant thereof was collected and 200 μl of chloroform was added thereto. The resultant mixture was vortexed and centrifuged at 12,000 rpm for 10 minutes at room temperature. After a supernatant was collected, 3.3 μl of 3M sodium acetate and 2.0 μl of Ethachinmate were added to the supernatant and the resultant mixture was vortexed. 400 μl of ice-cooled ethanol (EtOH) was further added, and the resultant mixture was vortexed and centrifuged at 12,000 rpm for 5 minutes at 4° C. After EtOH was removed, 200 μl of 80% EtOH was added and the resultant mixture was centrifuged at 15,000 for 30 seconds at 4° C. Then, EtOH was removed and the resultant mixture was air-dried. Thereafter, the obtained product was dissolved in 30 μl of RNase-free water for reverse transcription reaction.

The enzyme used for reverse transcription reaction was ThermoScript RT-PCR System. 9 μl of the obtained RNA solution and 1 μl of primers (oligo(dT)₂₀ 50 μM) were put into a 0.5 ml Eppendorf tube and incubated at 60° C. for 5 minutes. Then, 4 μl of 5×cDNA Synthesis Buffer, 1 μl of 0.1 M DTT, 1 μl of RNase OUT, 1 μl of DEPC-treated water, 2 μl of 10 mM dNTP Mix, and 1 μl of Thermoscript RT were added to the incubated mixture and the resultant mixture was incubated at 55° C. for 60 minutes. Then, after the resultant mixture was further incubated at 85° C. for 5 minutes, 1 μl of RNase H was added and the resultant mixture was incubated at 37° C. for 5 minutes, thereby preparing cDNAs. At the same time, in order to confirm that no amplification occured from genome DNAs, templates (RNA) that do not cause reverse transcription reaction were also prepared. As templates, cDNAs derived from RNAs of each organ and RNAs were used to perform PCR on each RIKEN Clone. HotStarTaq DNA Polymerase was herein used. As described below, two reaction systems were prepared for the operation.

(1) PCR Using cDNA as Template

2.5 μl of 10×PCR Buffer, 5 μl of 5× Q-solution, 2.5 μl of d-NTP Mix (2 mM), 0.125 μl of HotStarTaq DNA Polymerase, 6.9 μl of dH₂₀, 1 μl of template DNA (synthesized cDNA), 2.5 μl forward primer (5 μM), and 2.5 μl of reverse primer (5 μM) were placed into a PCR tube (25 μl in total). The reaction conditions for PCR were: heating at 95° C. for 15 minutes; 35 cycles of 1 minute at 94° C., 1 minute at 55° C., and then 1 minute at 72° C.; and thereafter a reaction at 72° C. for 10 minutes.

(2) PCR Using RNA as Template

PCR was herein performed under the same conditions as described above (1). Instead of 1 μl of template DNA (synthesized cDNA), 1 μl of RNA that has been treated with DNase but just before cDNA synthesis was used.

4 μl of the PCR products was subjected to 2.5% agarose gel electrophoresis, stained with ethidium bromide (EtBr), and photographed under UV-irradiation.

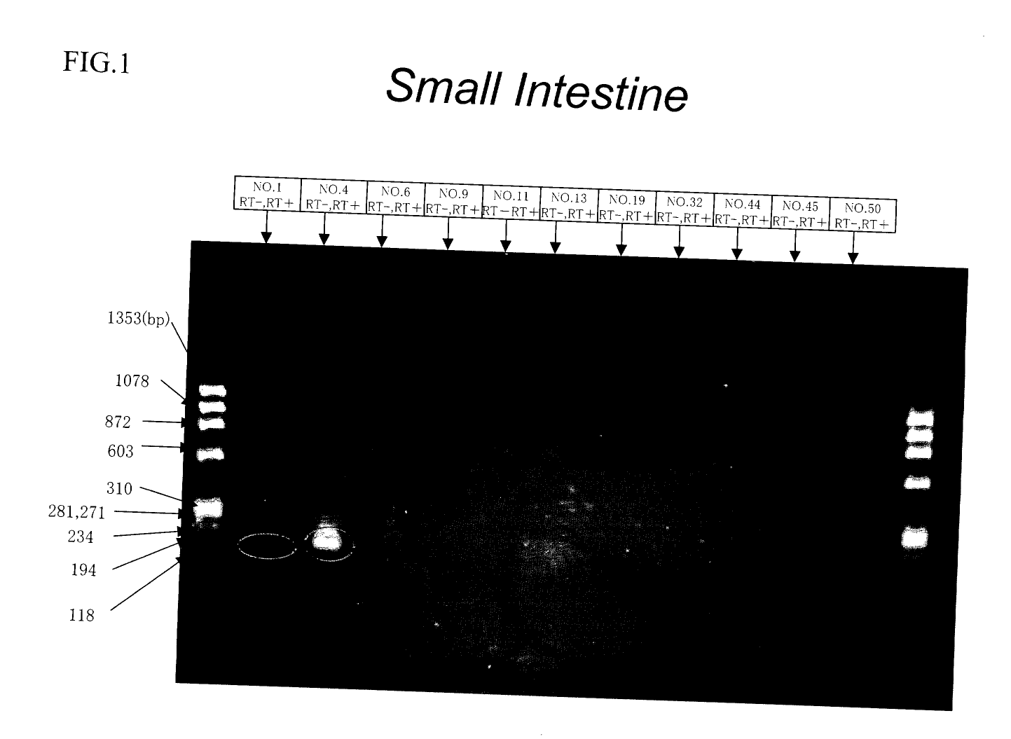

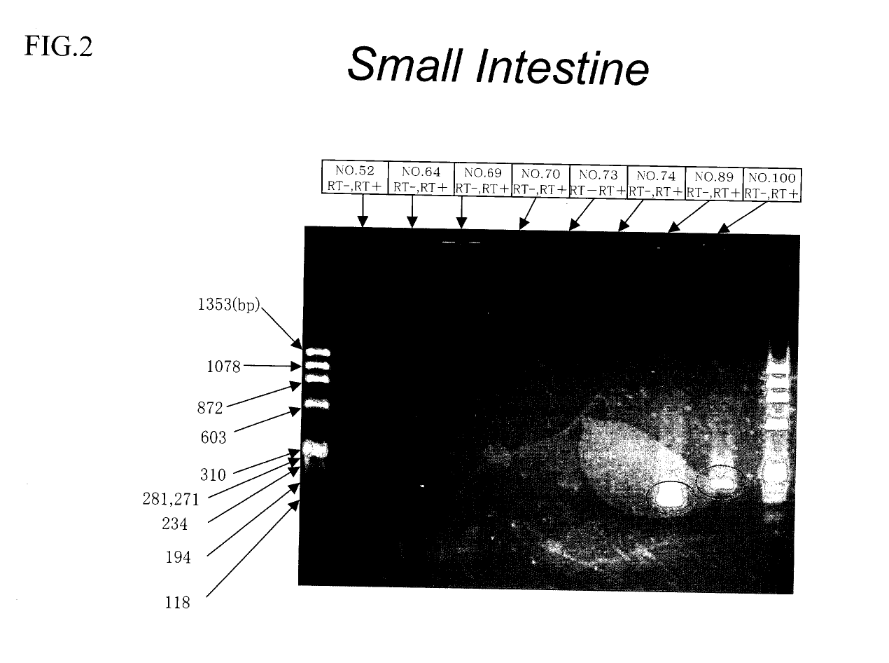

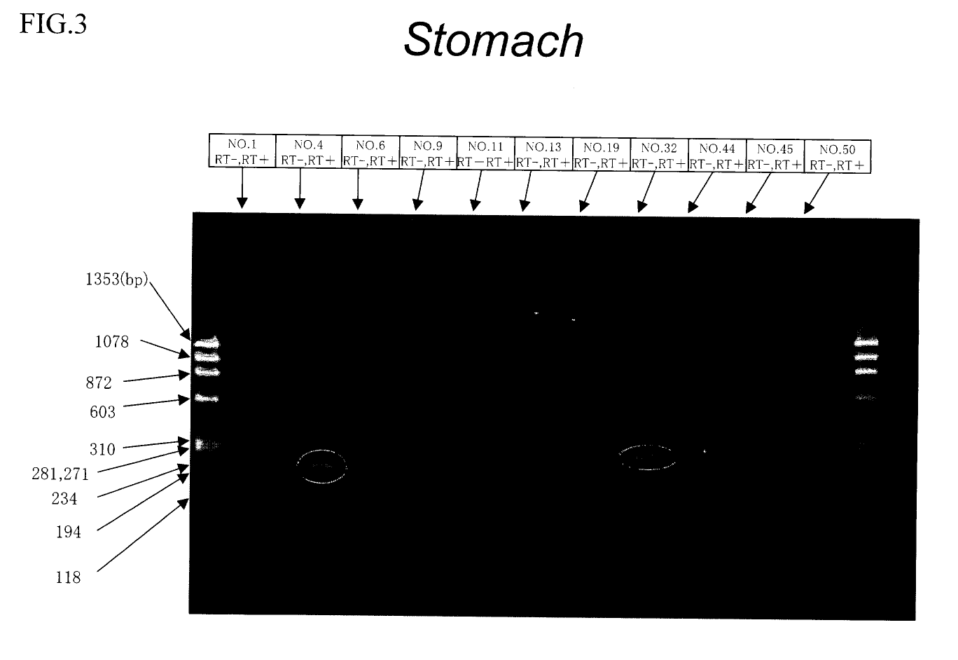

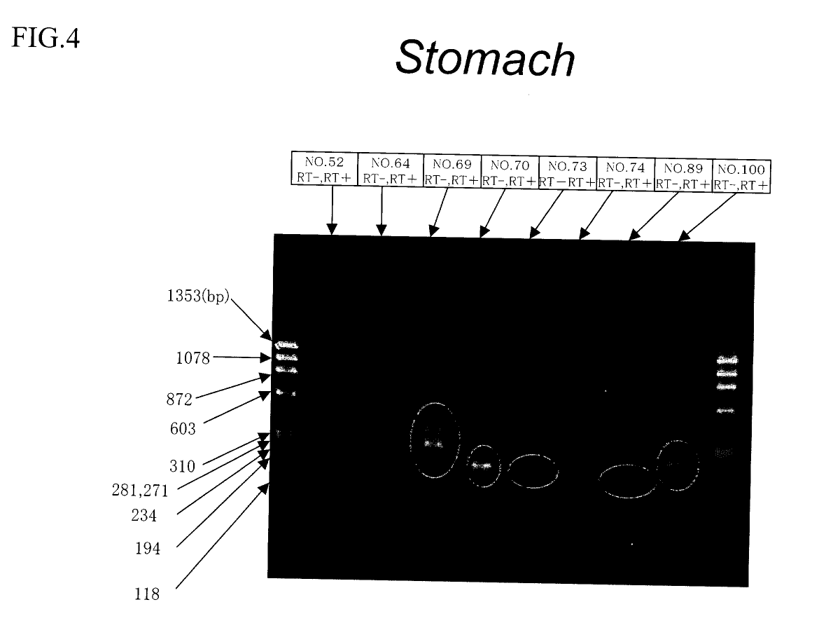

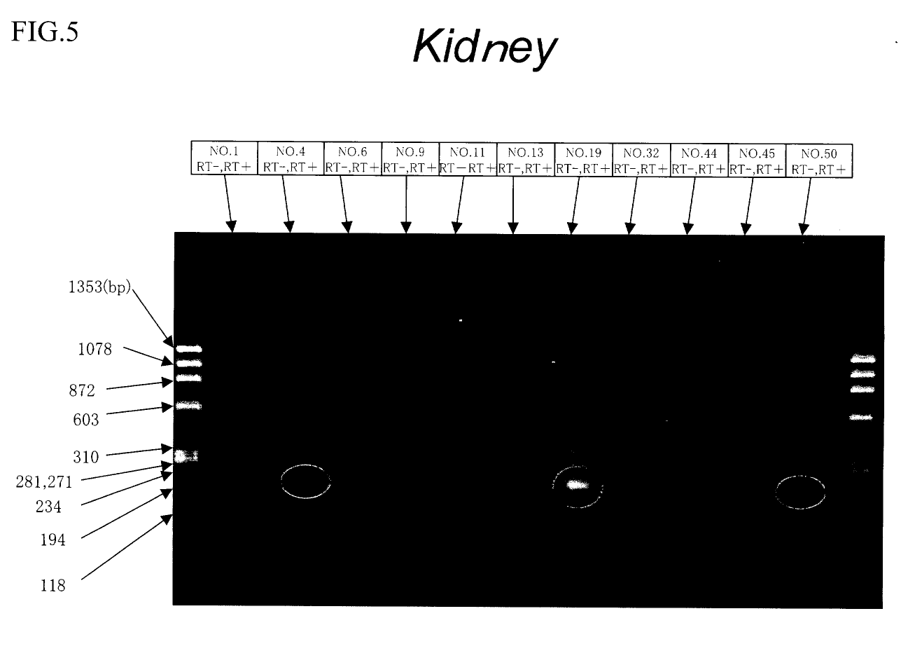

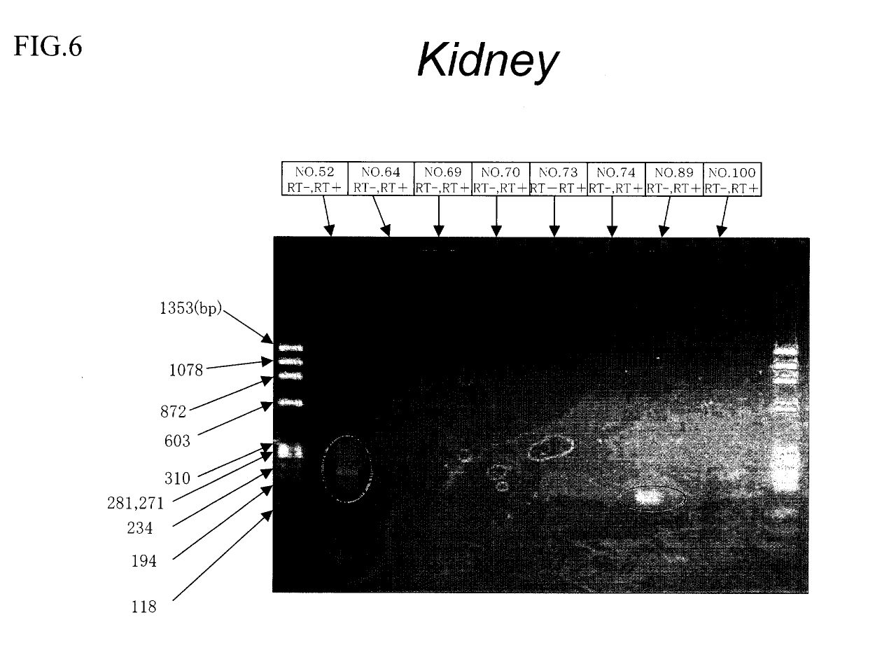

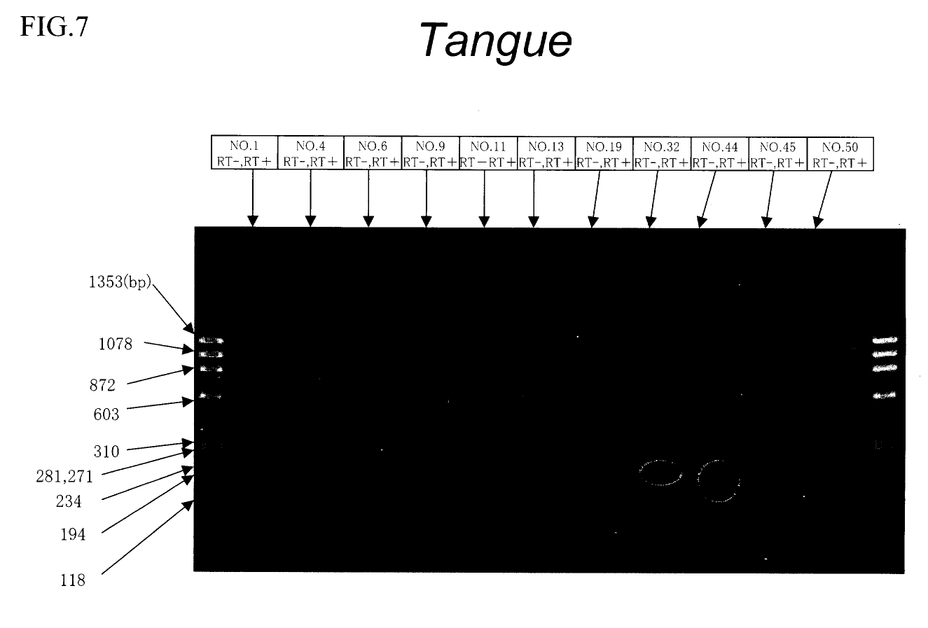

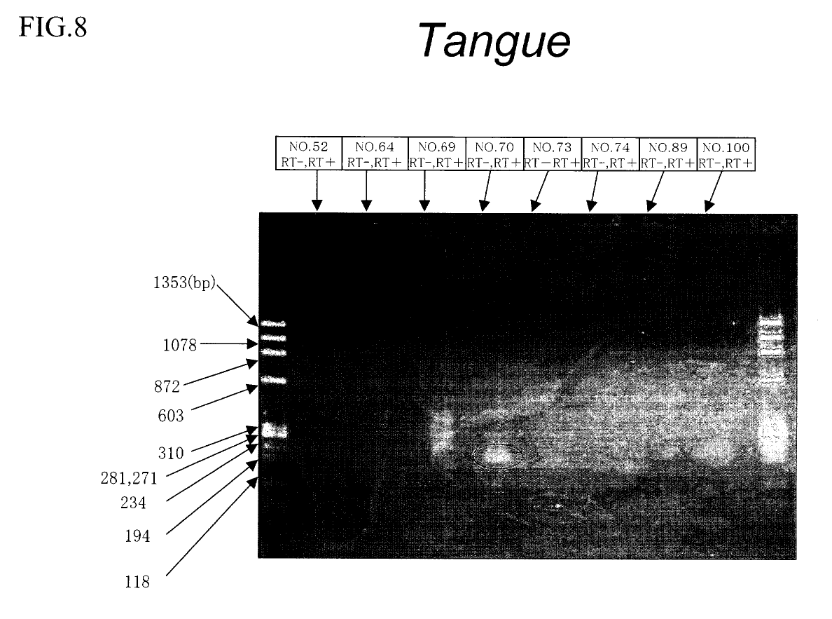

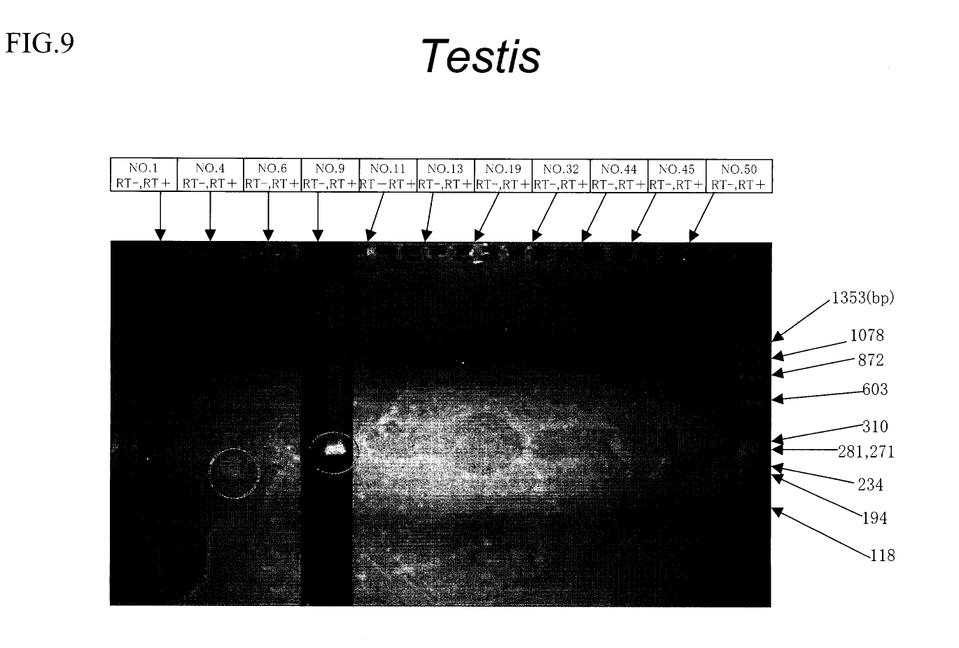

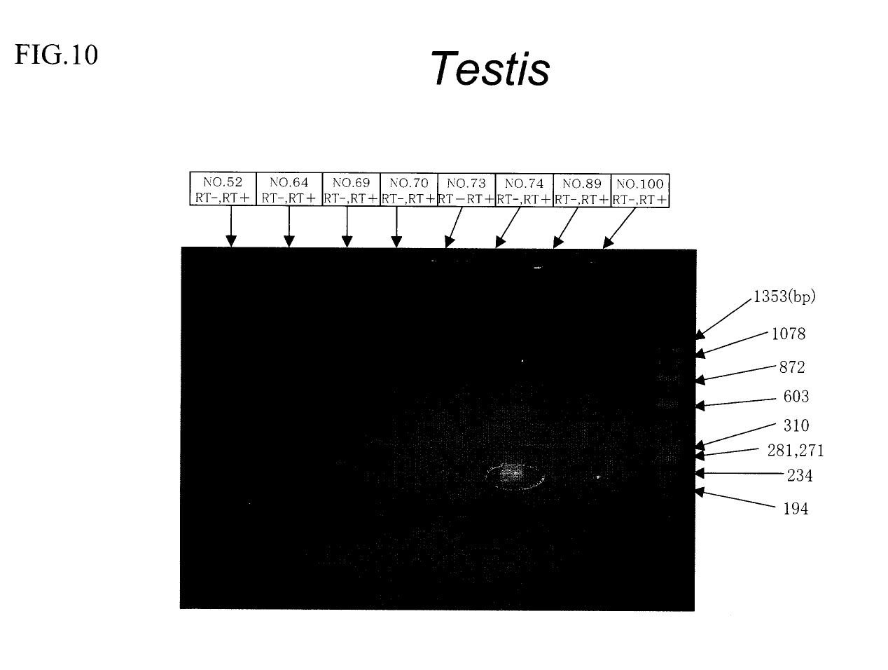

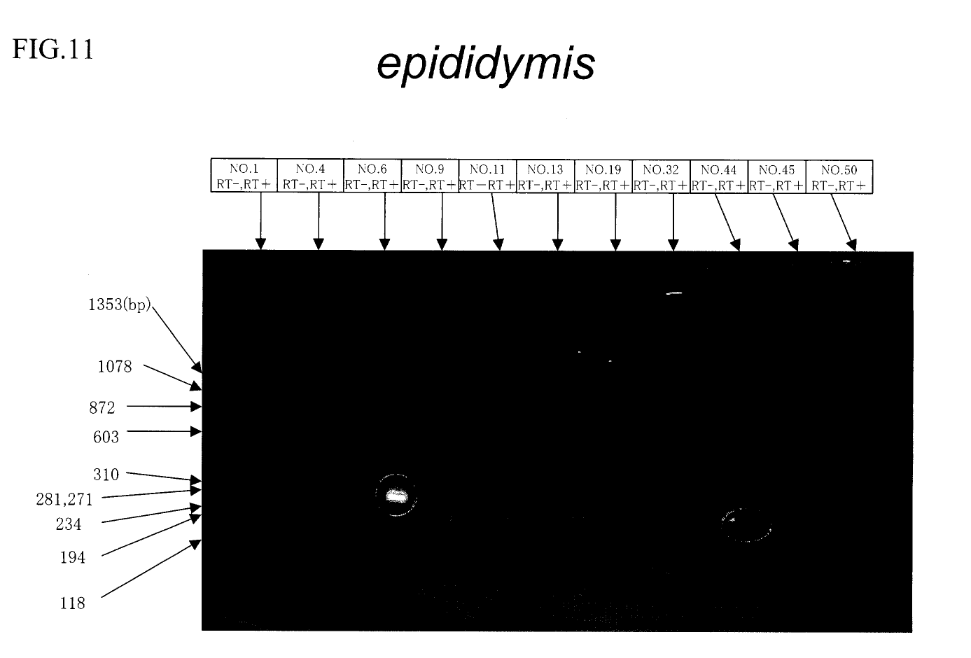

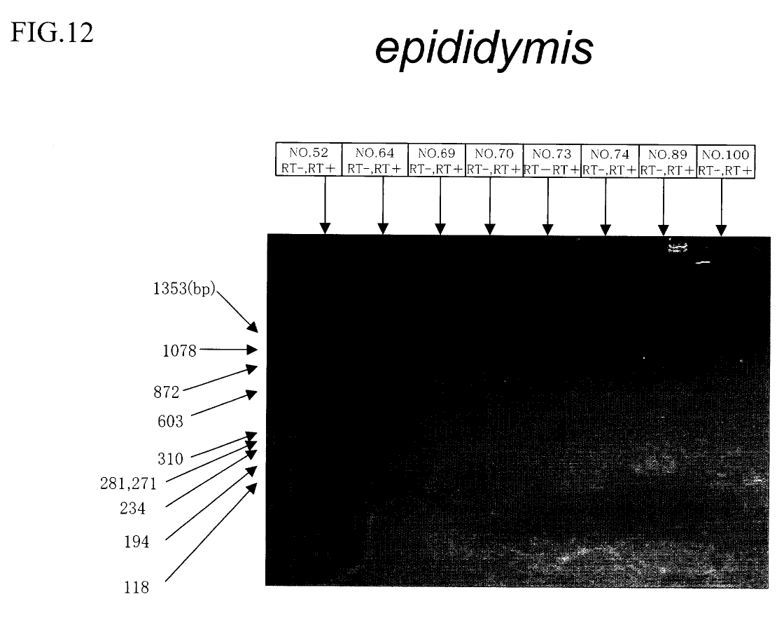

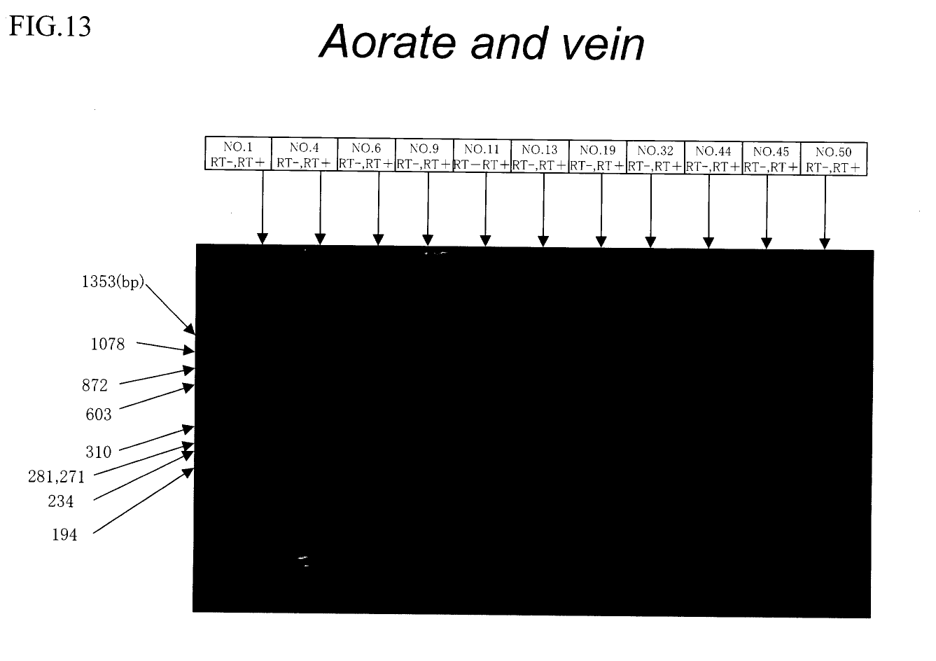

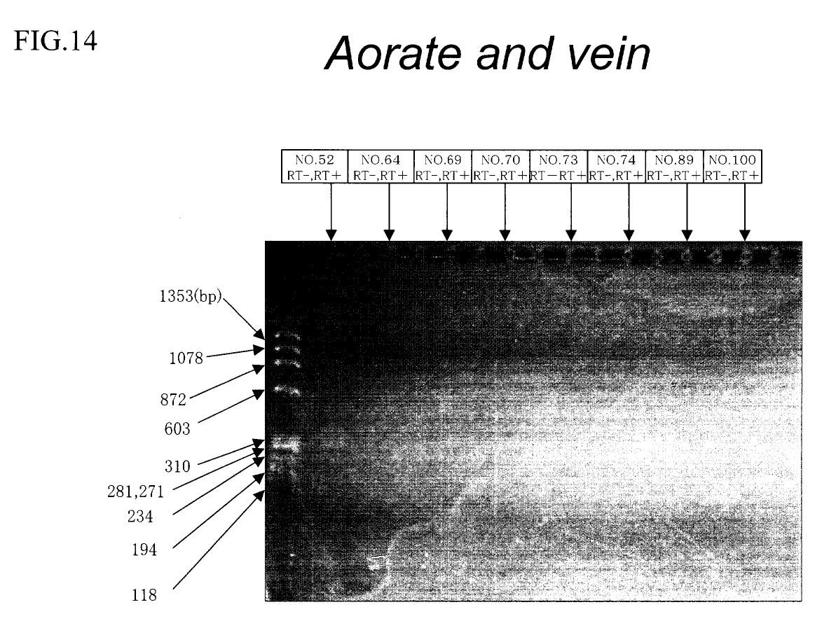

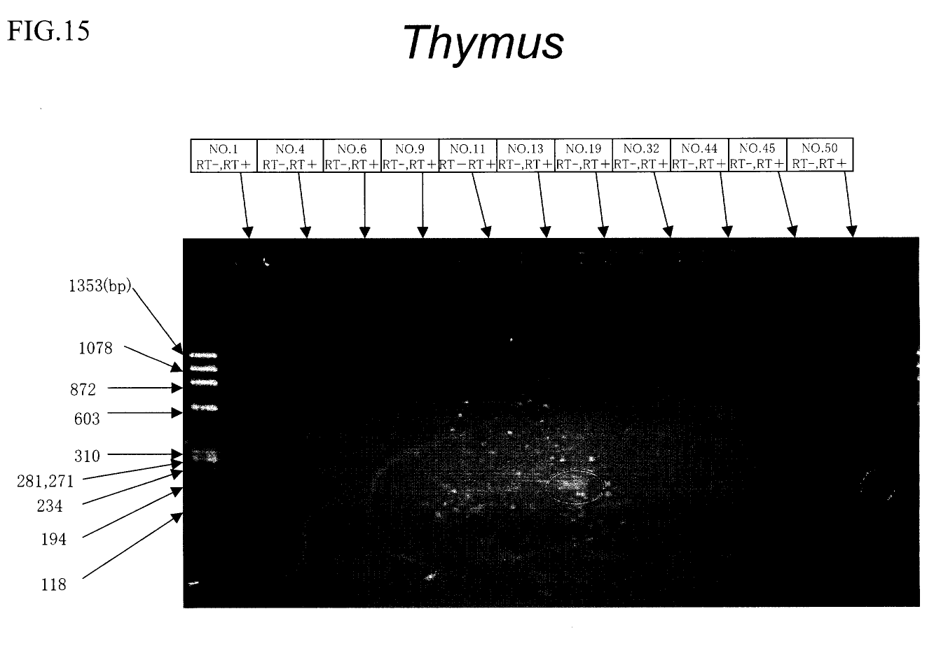

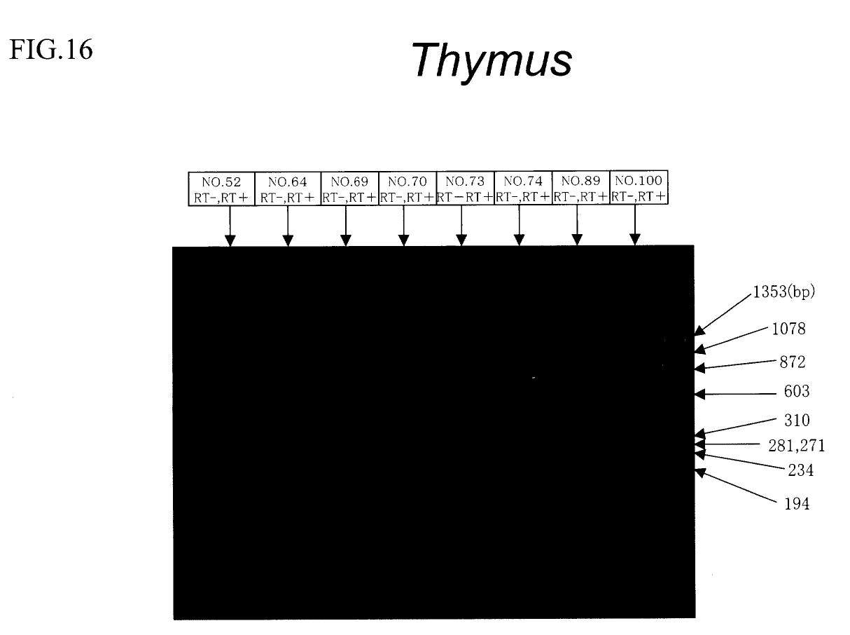

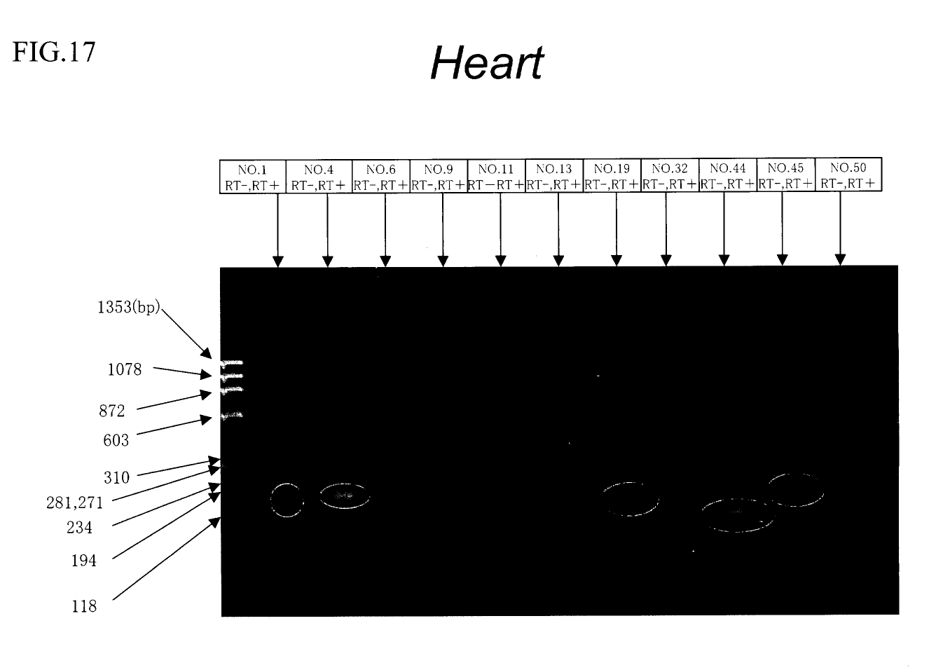

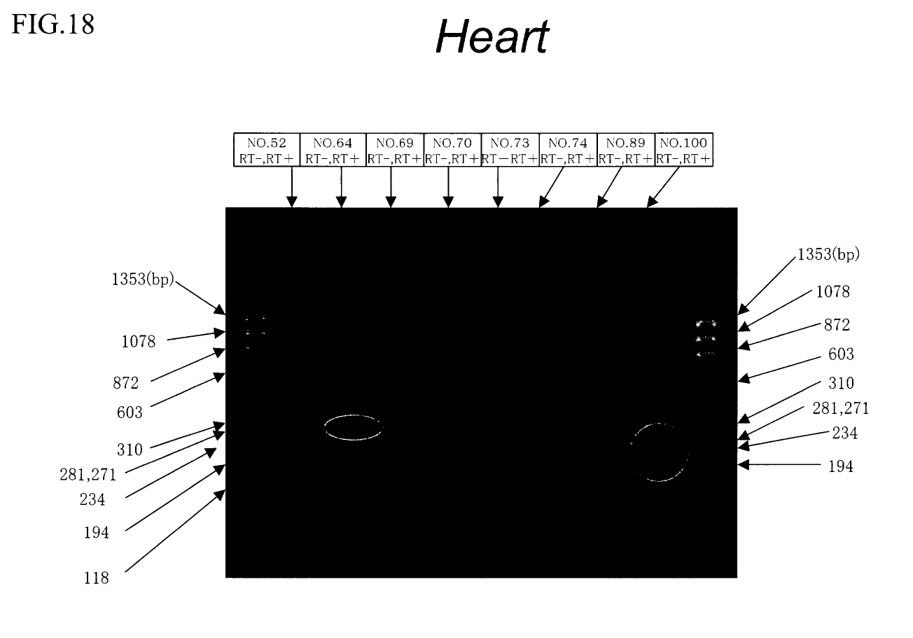

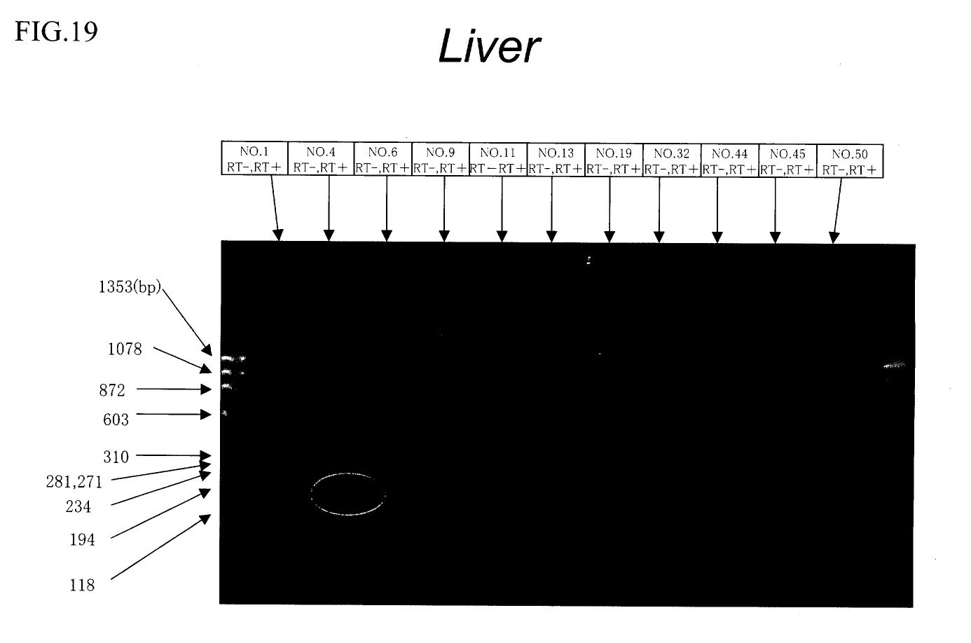

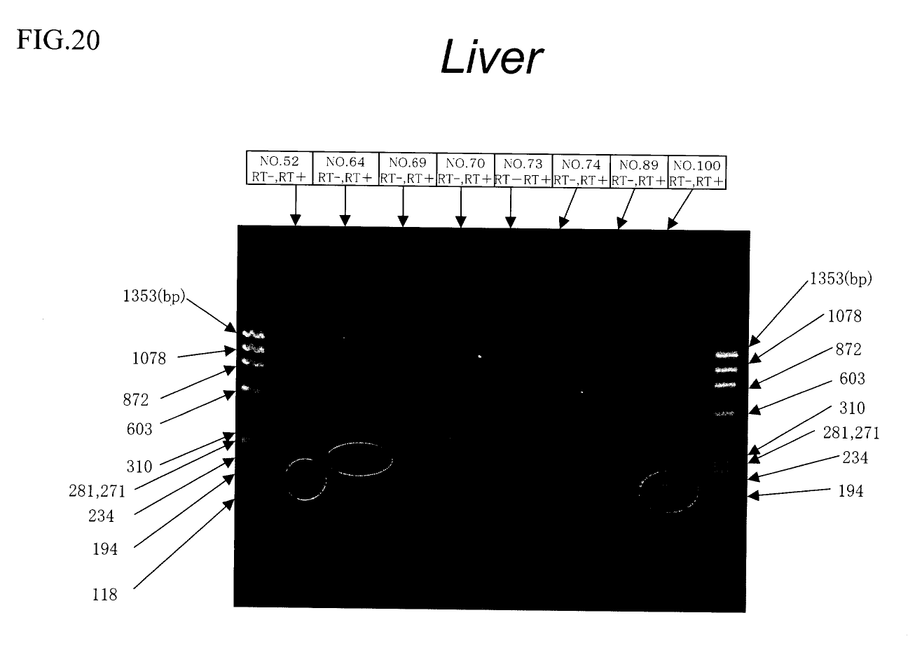

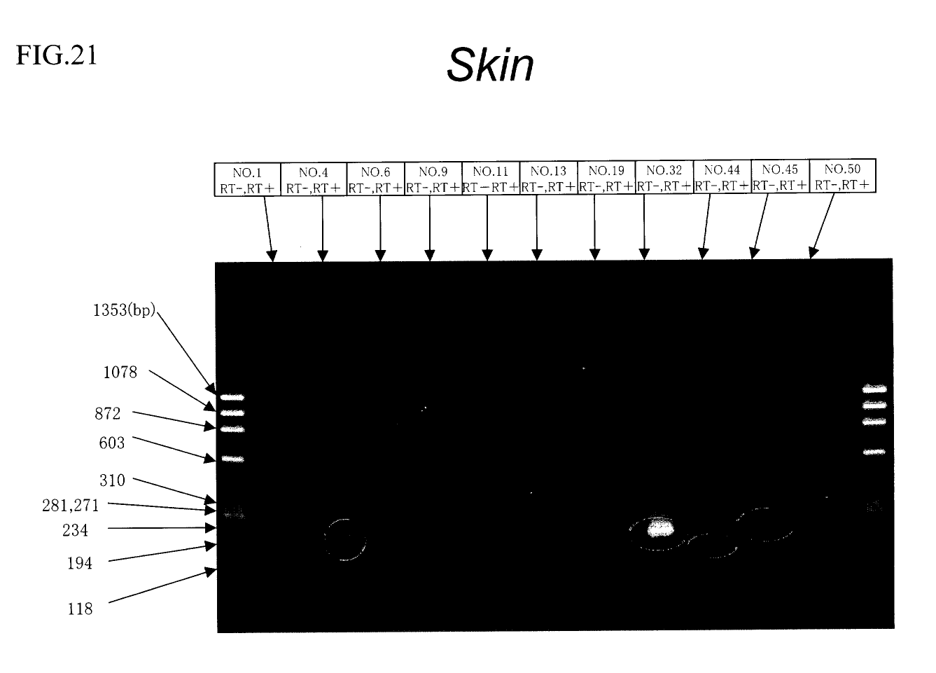

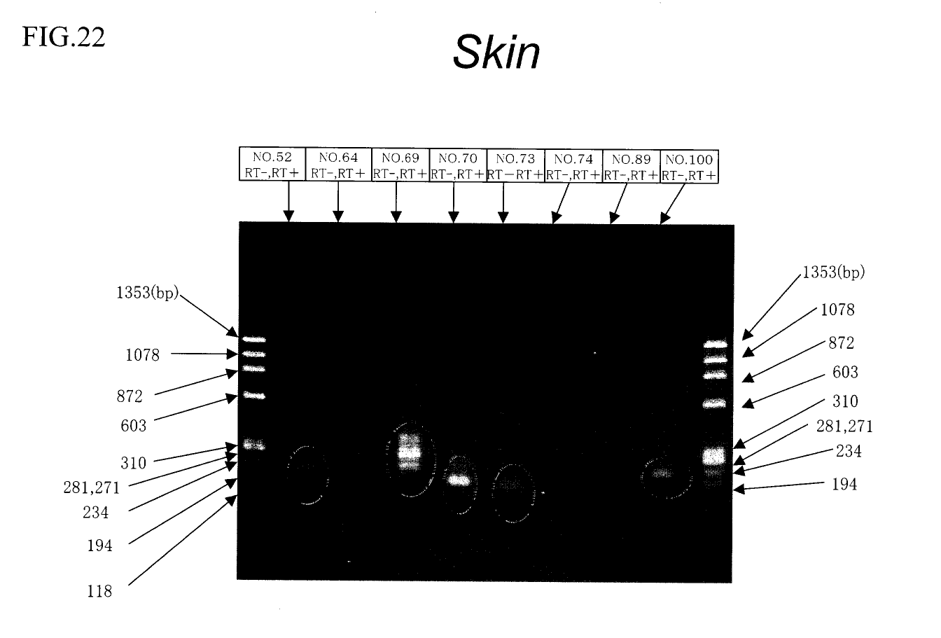

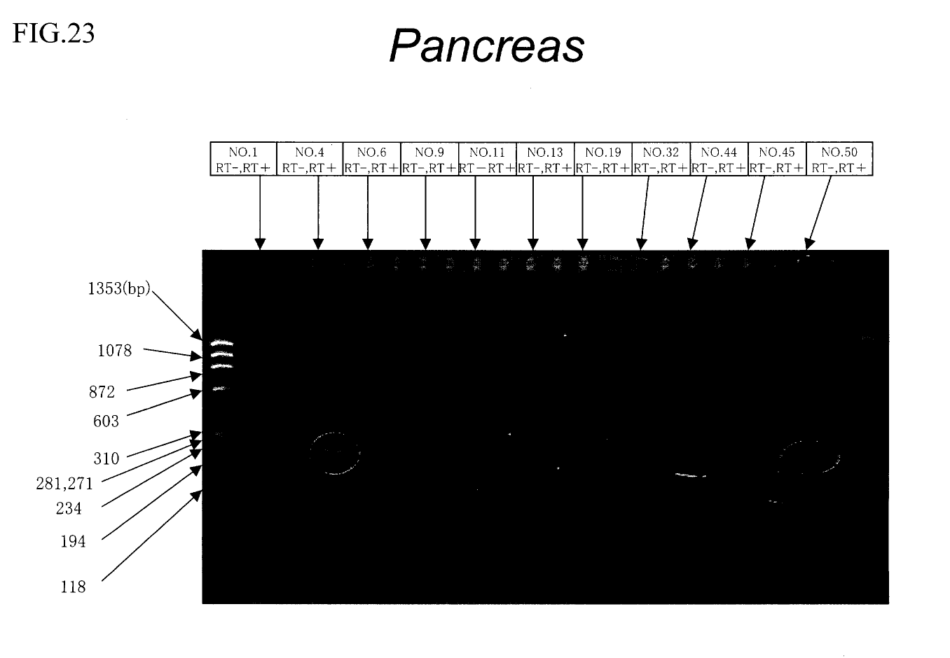

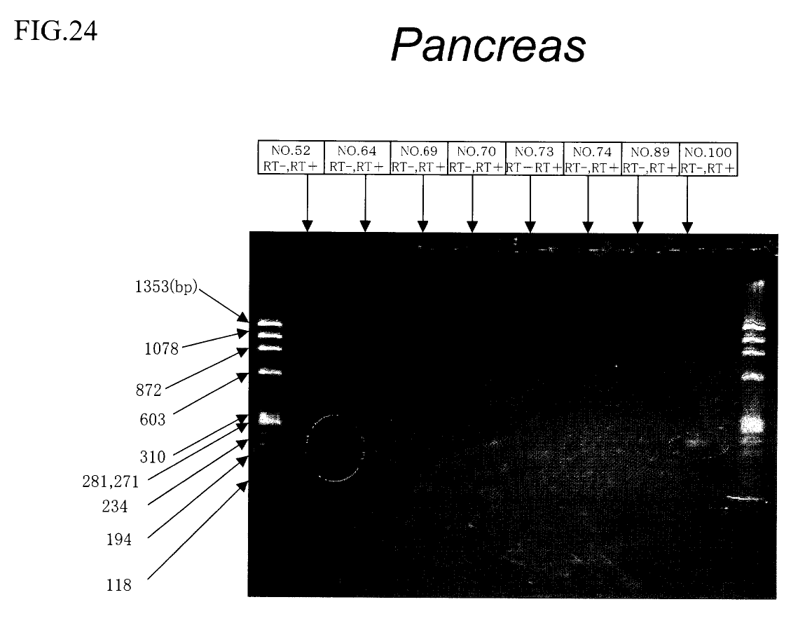

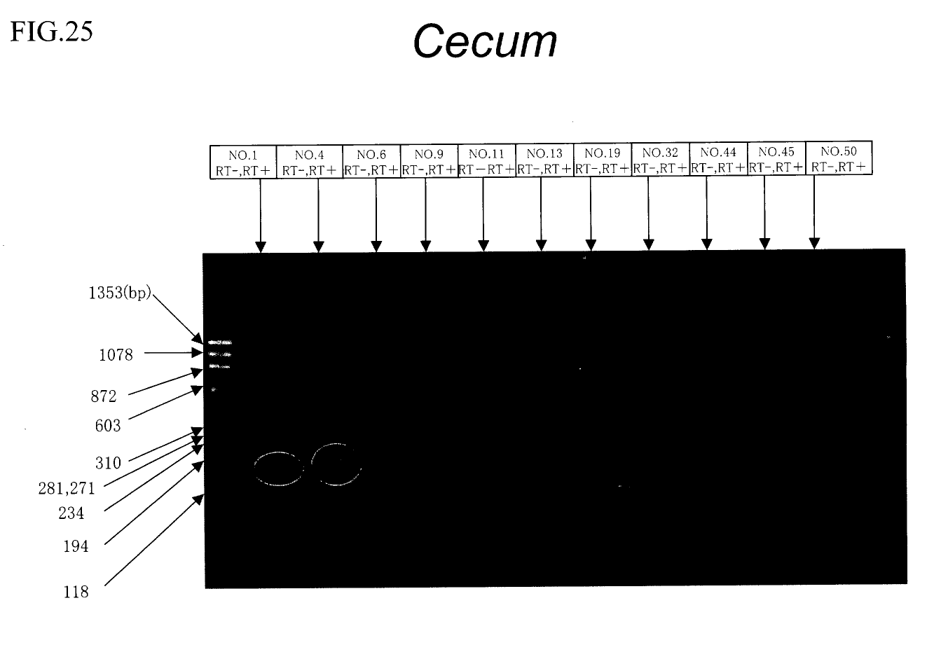

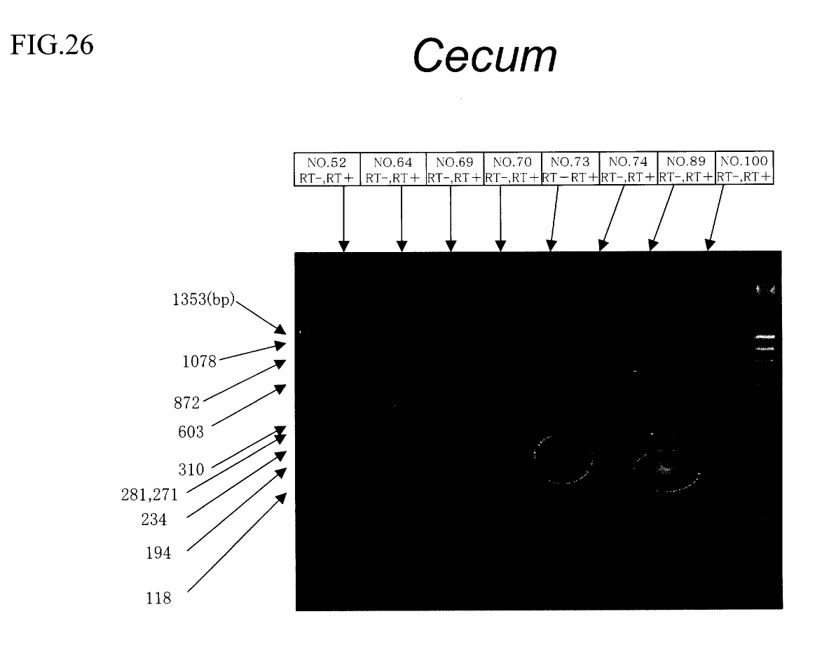

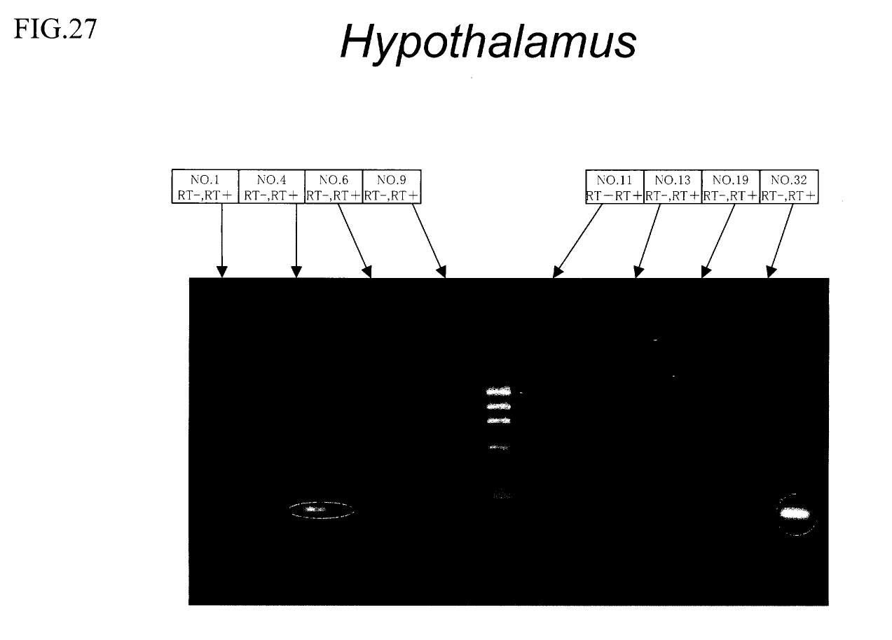

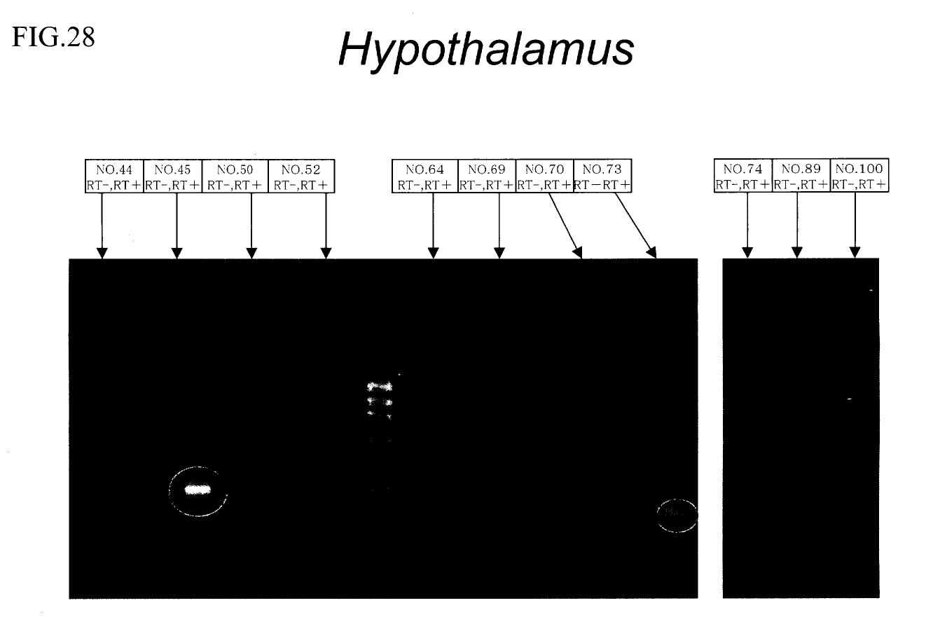

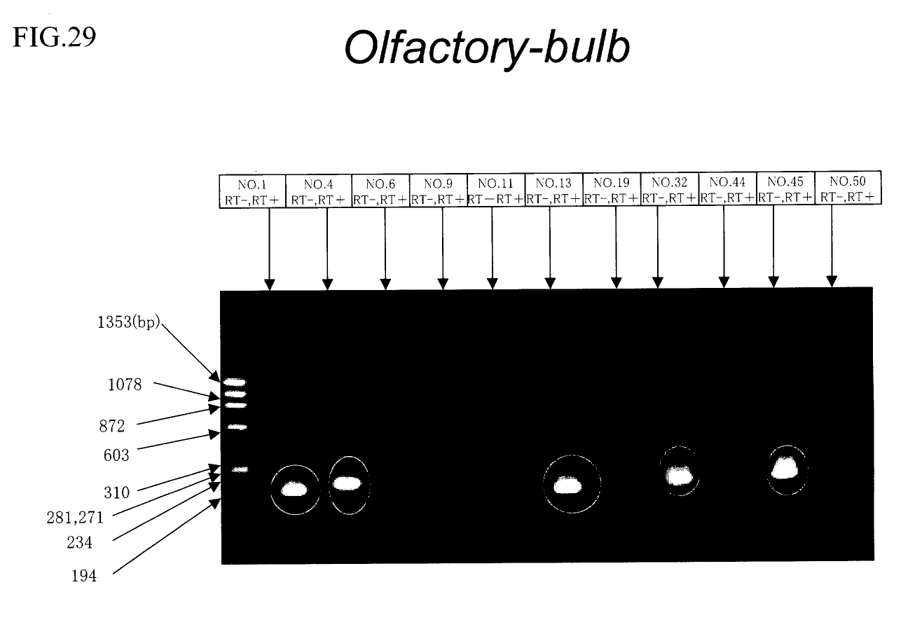

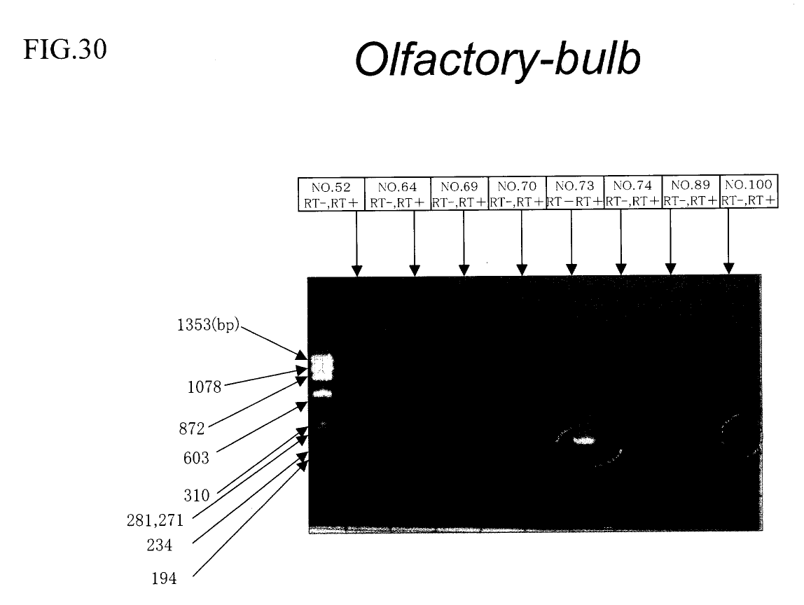

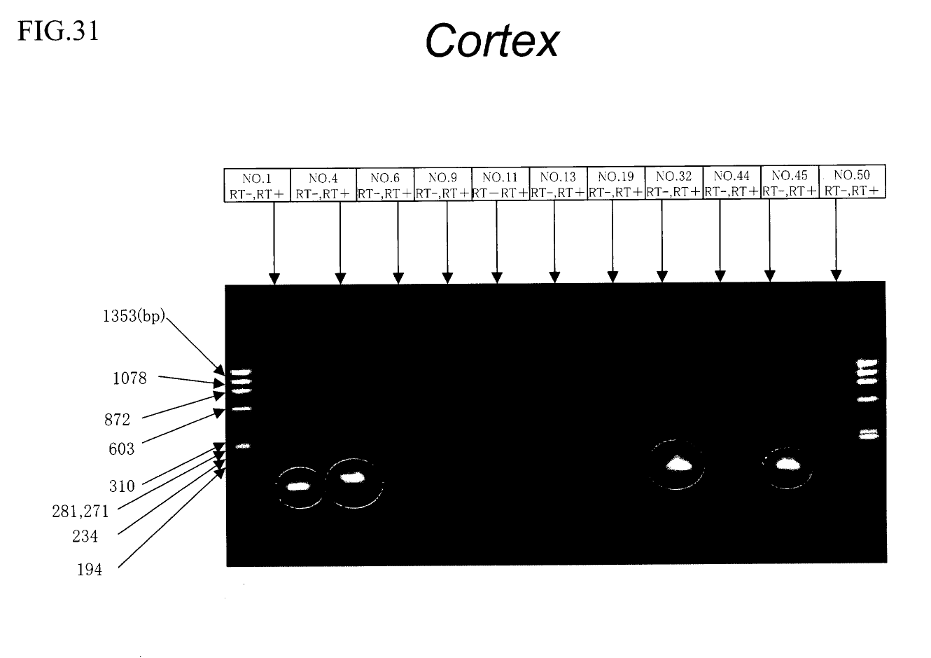

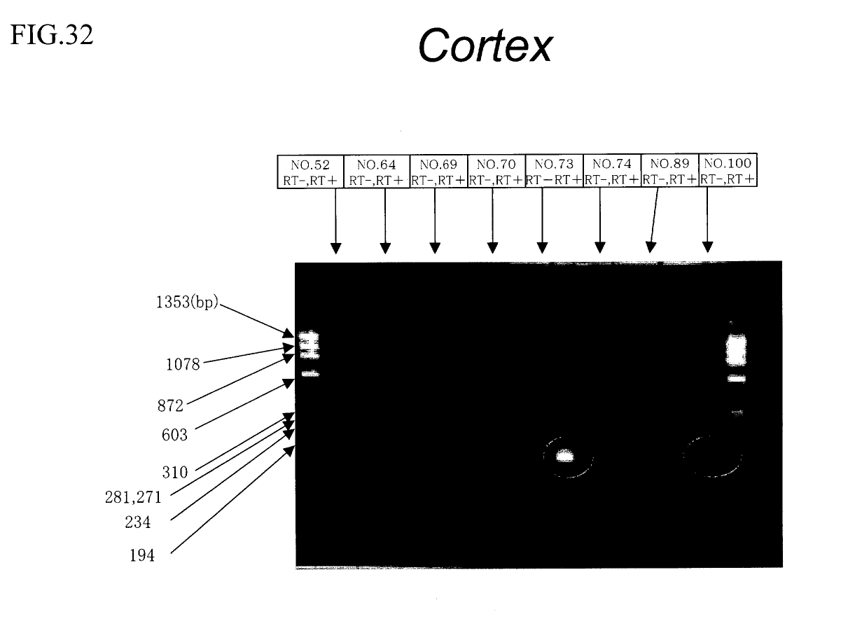

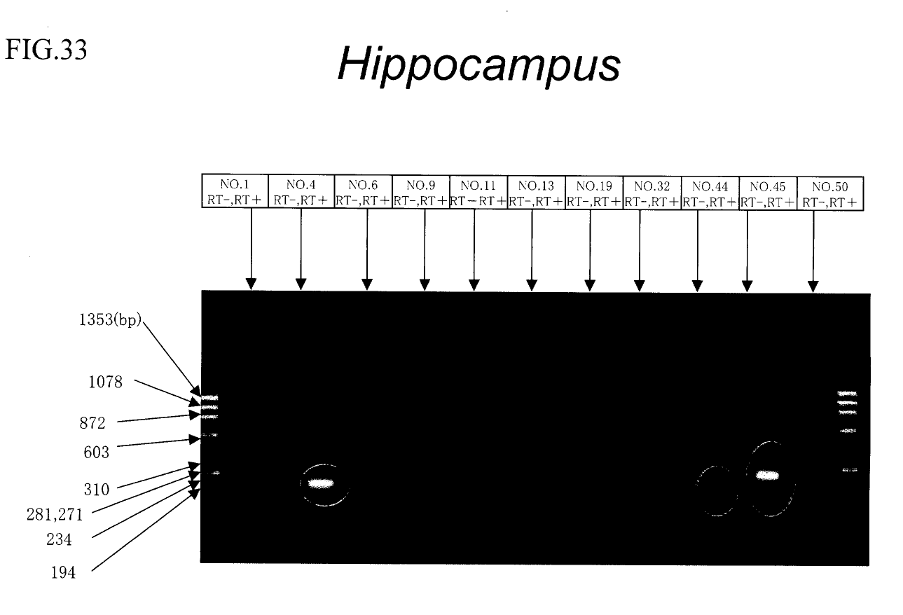

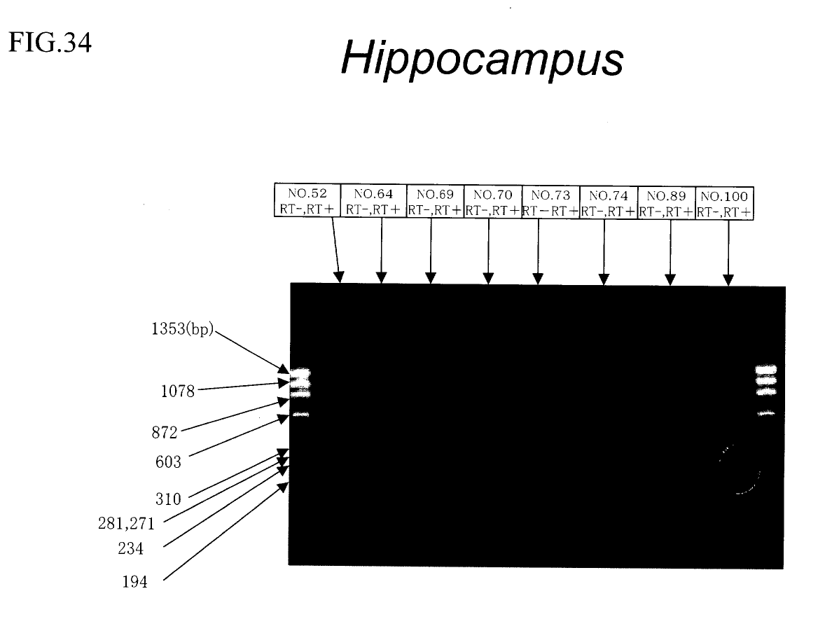

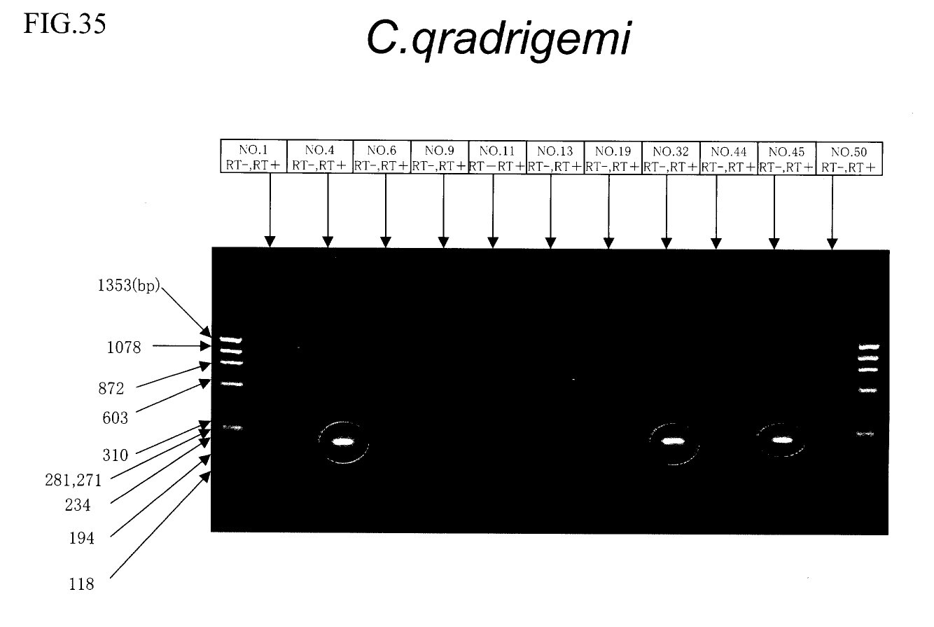

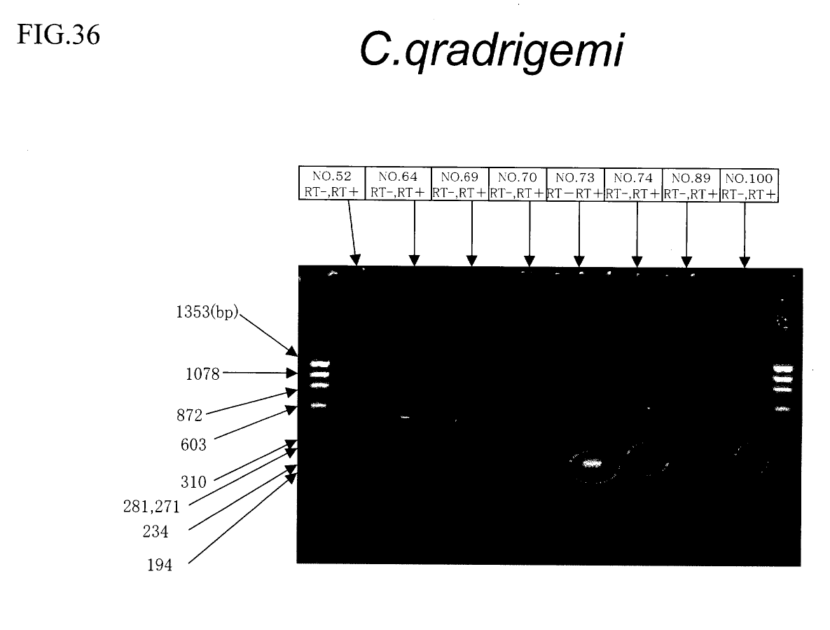

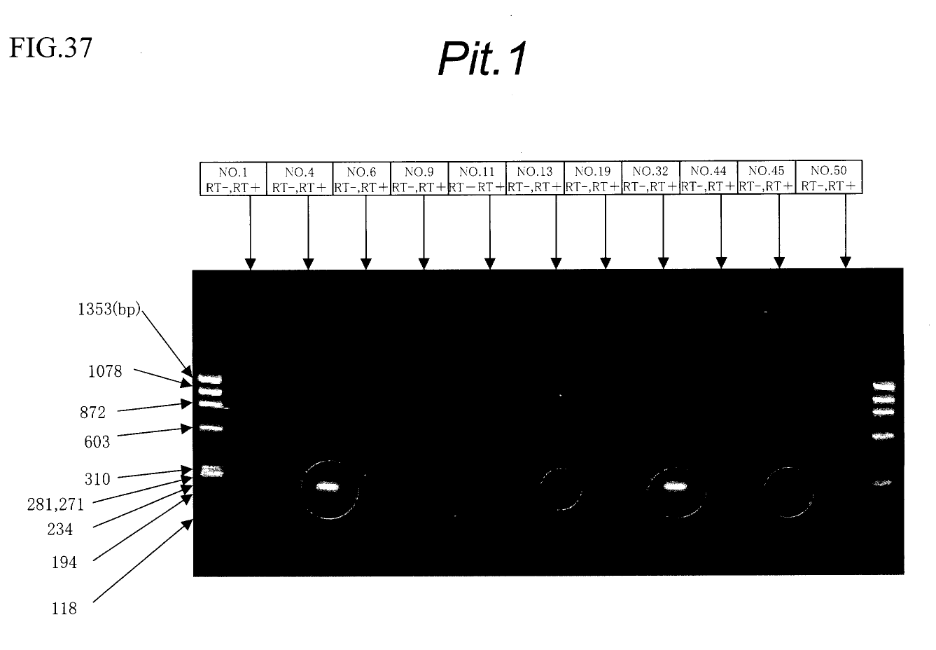

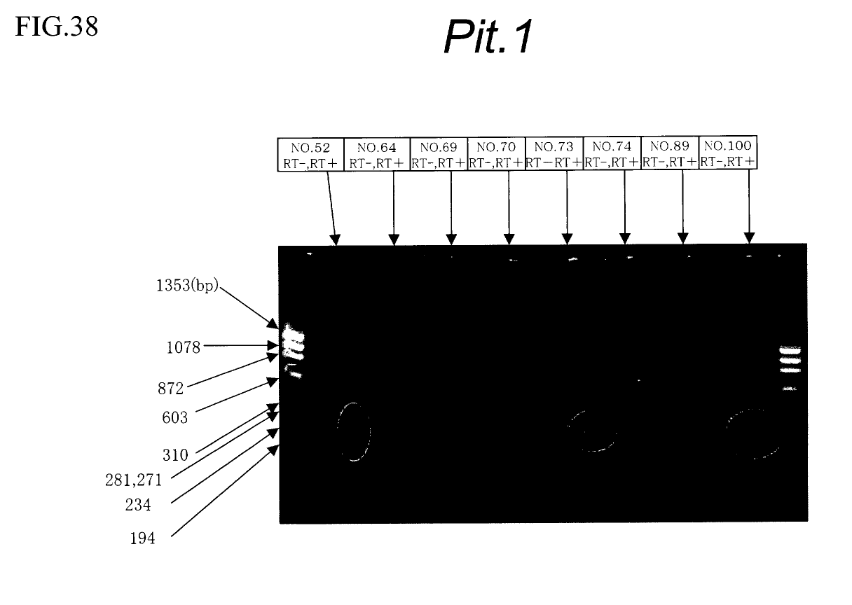

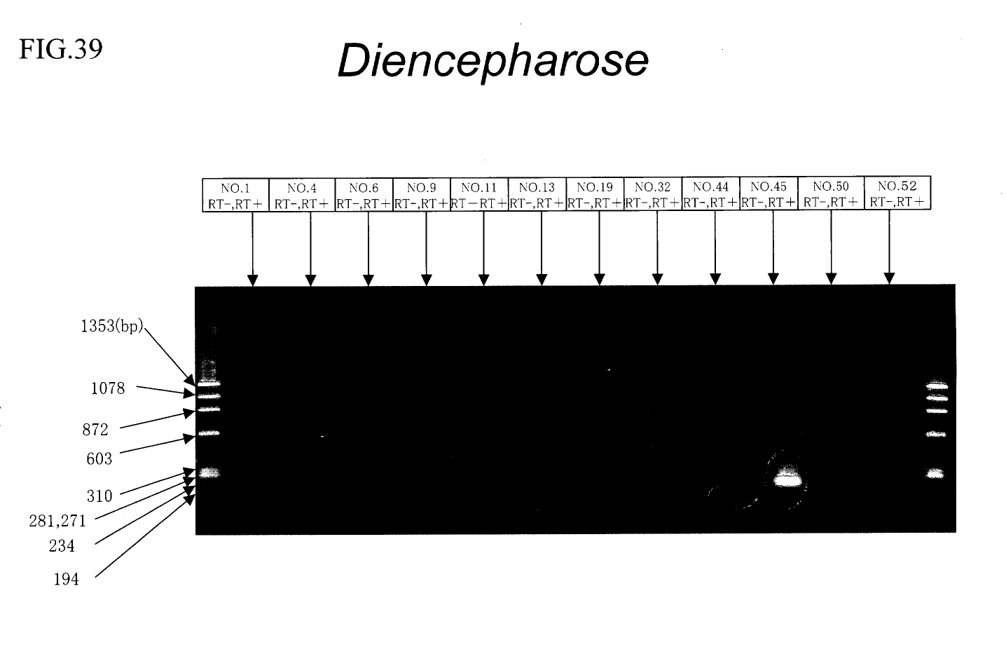

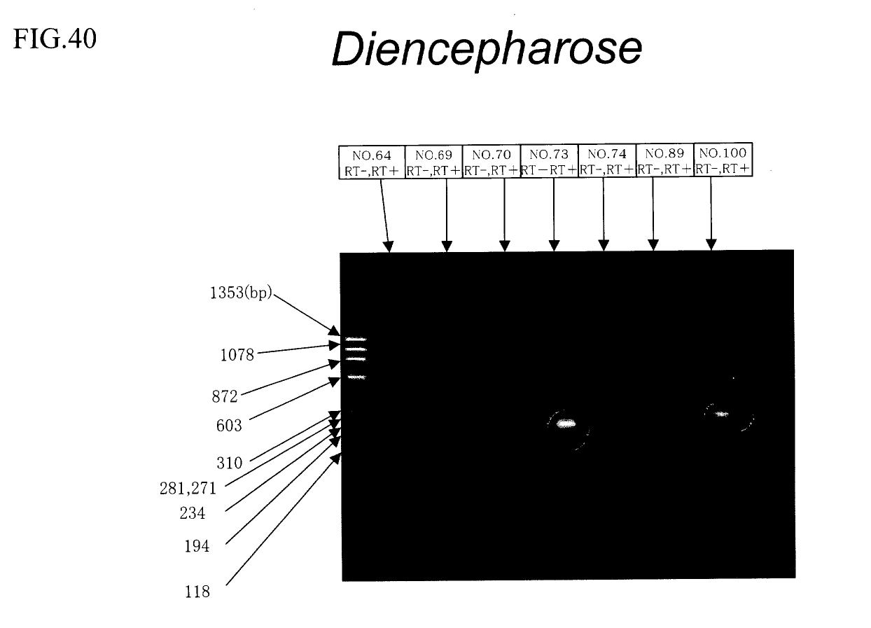

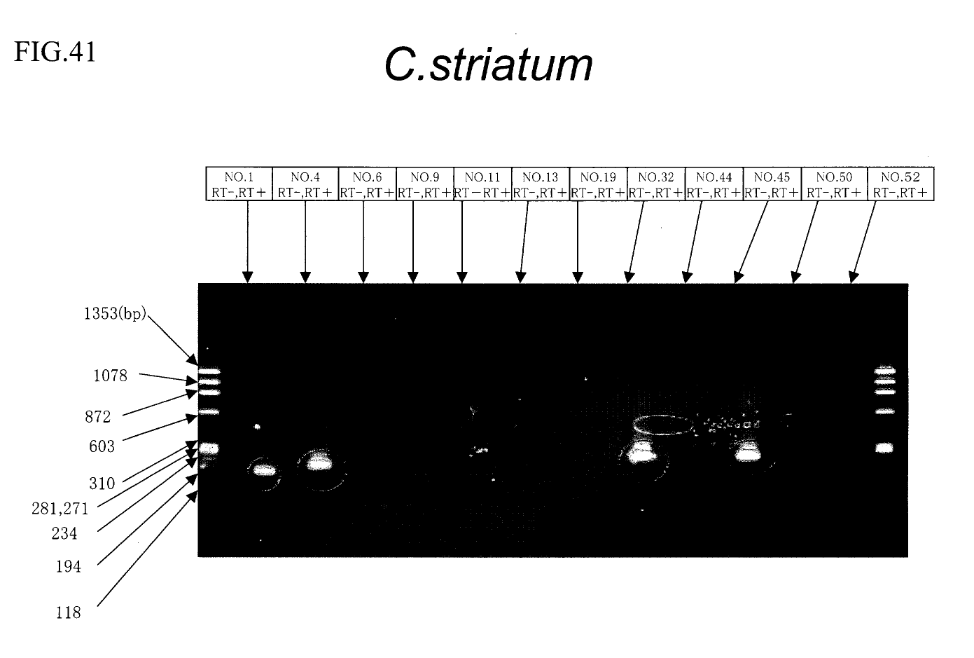

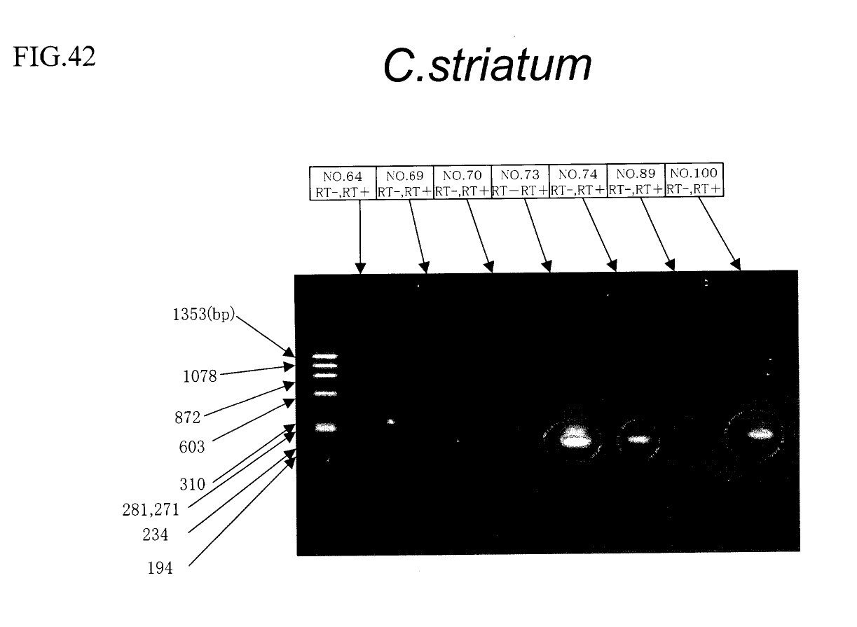

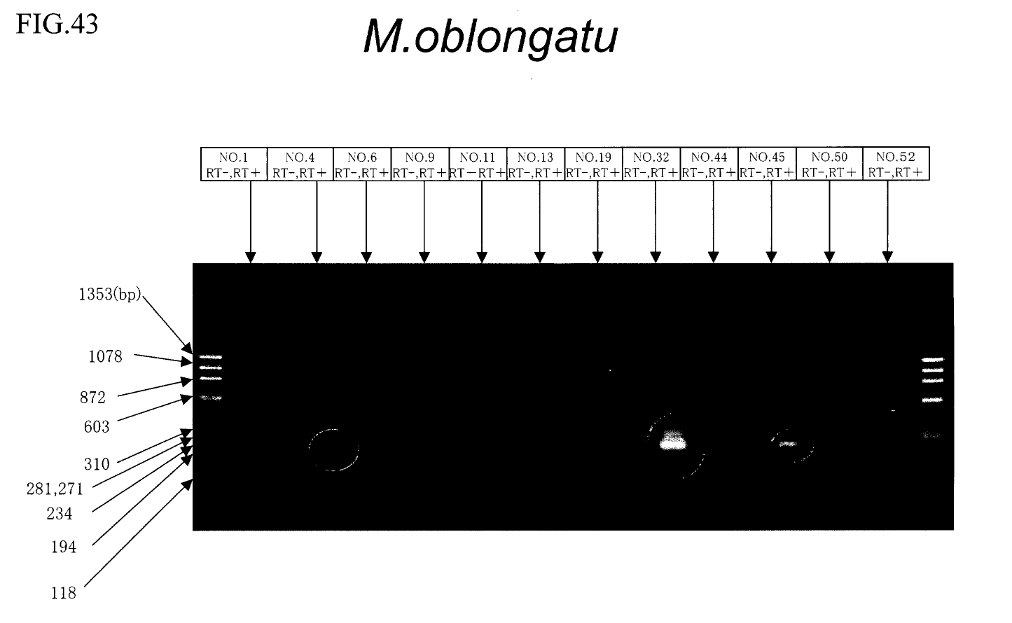

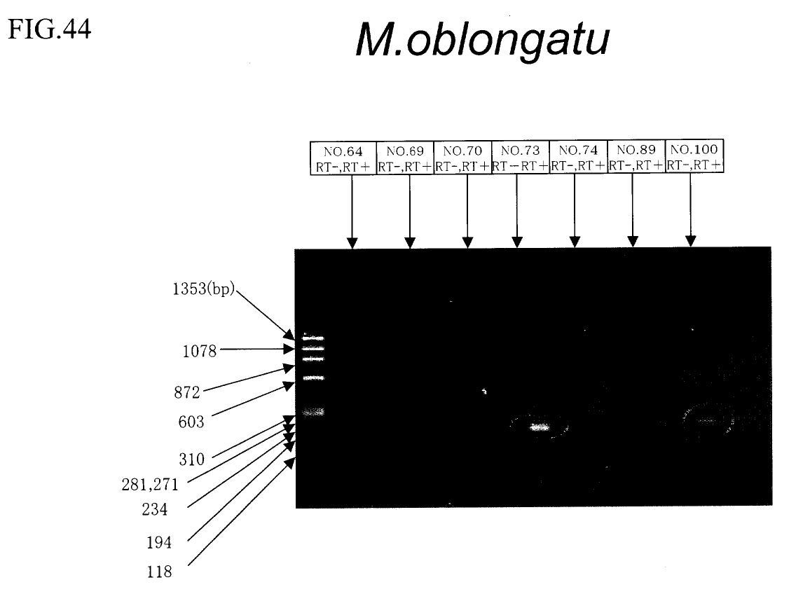

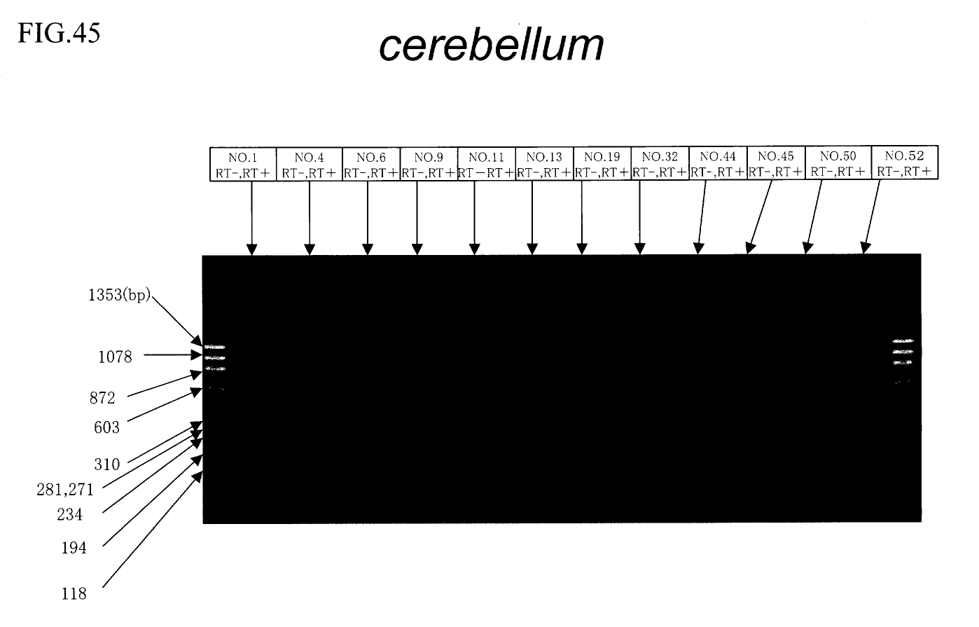

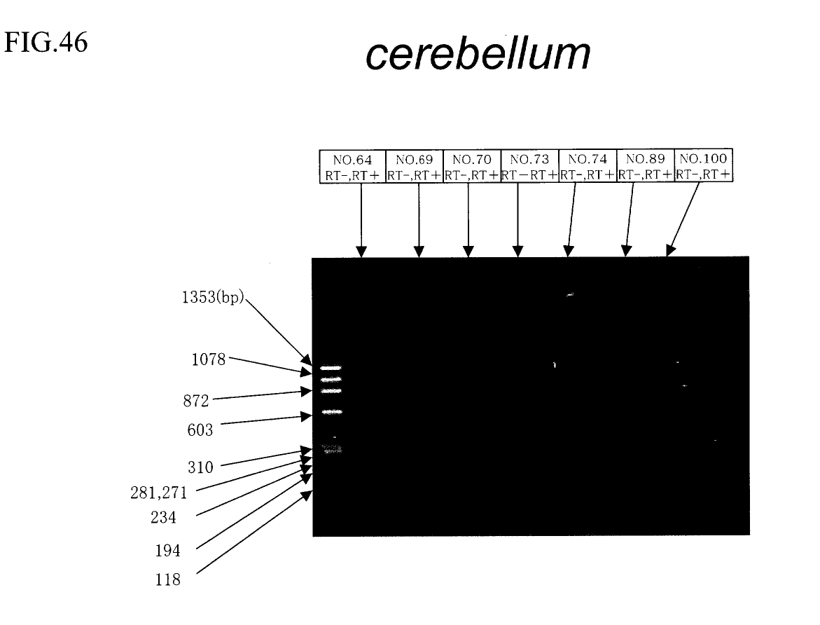

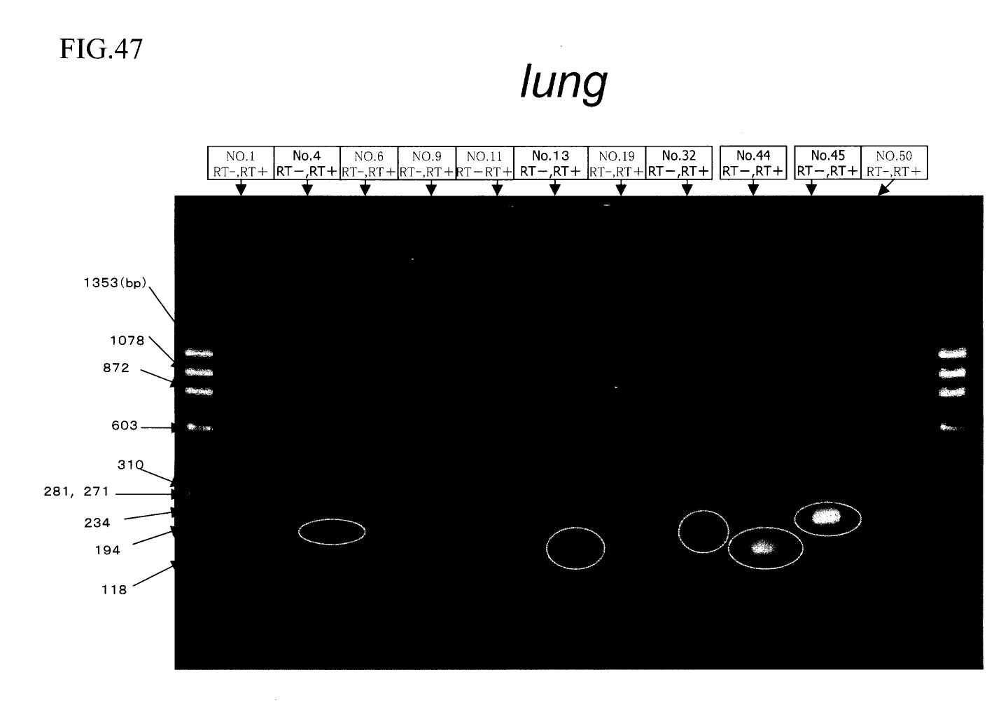

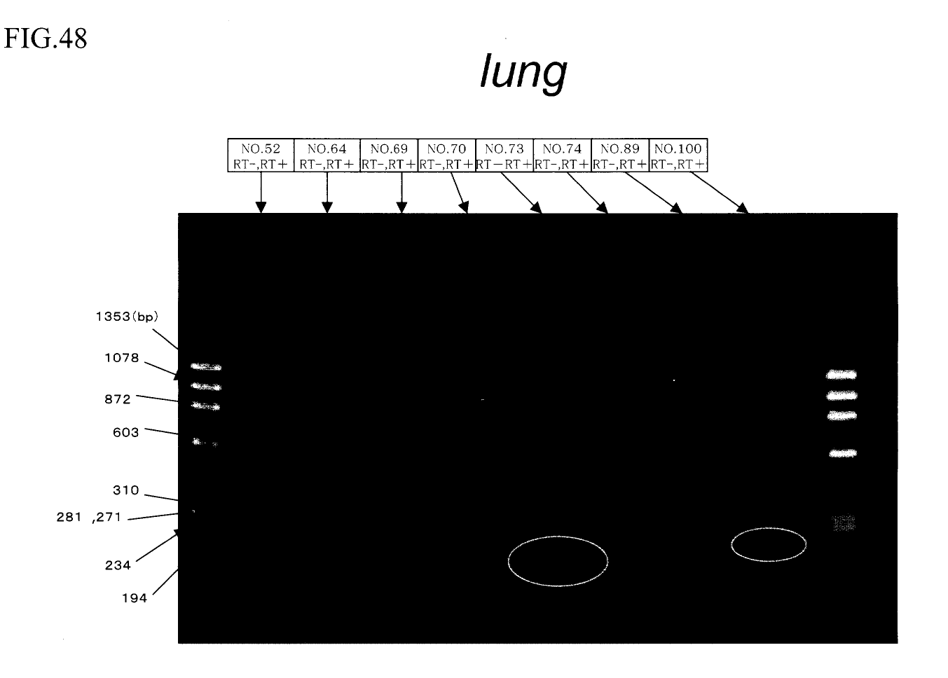

The resulted photographs are shown in FIGS. 1 to 48. Tables 3-1 to 3-3 show the transcription pattern in various organs. In tables 3-1 to 3-3, a circle means that the clone is transcribed in the indicated organ.

TABLE 3-1
Transription pattern for each clone in various organs

| | Primer | | | | | | | |
|---|---|---|---|---|---|---|---|---|
| | 1 | 4 | 6 | 9 | 11 | 13 | 19 | 32 |
| | Clone ID | | | | | | | |
| | A830010B16 | C230071E12 | 9230111O07 | 1700056N09 | 9230110A19 | A430045L05 | 4930563B01 | A030004E11 |
| | | | | | | | | hypothetical |
| | | | | | | | | Prokaryotic |
| | | | | RIKEN cDNA | | | | membrane lipoprotein |
| | unknown | unknown | | 170063l17 | | | | lipid attachment site |
| Curated Gene Name | EST | EST | unclassifiable | gene | NO DATA | NO DATA | NO DATA | containing protein |
| Hypothalamus | | ◯ | | | | | | ◯ |
| Olfactory bulb | ◯ | ◯ | | | | ◯ | | ◯ |
| C. qradrigemi | | ◯ | | | | | | ◯ |
| pit 1 | | ◯ | | | | | ◯ | ◯ |
| Cortex | ◯ | ◯ | | | | | | |
| Diencepharose | | | | | | | | |
| Corpus striatum | ◯ | ◯ | | | | | | ◯ |
| Hippocampus | | ◯ | | | | | | |
| Cerebellum | | | | | | | | |
| m. oblongatu | | ◯ | | | | | | ◯ |
| aorate and vein | | | | | | | | |
| thymus | | | | | | ◯ | | |
| cecum | ◯ | ◯ | | | | | | |
| epididymis | | | ◯ | | | | | |
| testis | | ◯ | | ◯ | | | | |
| tongue | | | | | | | | ◯ |
| kidney | | ◯ | | | | | ◯ | |
| skin | | ◯ | | | | | | ◯ |
| lung | | ◯ | | | | ◯ | | ◯ |
| heart | ◯ | ◯ | | | | | ◯ | |
| liver | | ◯ | | | | | | |
| stomach | | ◯ | | | | | | ◯ |
| pancreas | | ◯ | | | | | | |
| small intestine | ◯ | ◯ | | | | | | |

Remarks:
Blank colums shows that the expression of the gene was not detected.
“NO DATA” shows that the information of the genes is not disclosed in FANTOM.
“Un known EST” and “unclassifiable” are the Curated Gene name disclosed in FANTOM.
In exprements, both of RT− and RT+ were performed. In most expreriments, bands were detected for RT+.
However, in the experiment using Primer 1, bands were detected for RT−.

TABLE 3-2
Transription pattern for each clone in various organs

| | Primer | | | | | |
|---|---|---|---|---|---|---|
| | 44 | 45 | 50 | 52 | 64 | 69 |
| | Clone ID | | | | | |
| | 1700007F22 | B230352O20 | D630020P16 | C630041L24 | 2310031C01 | 1110005I17 |
| | weakly similar to ACROSIN- | | | | | |
| | TRYPSIN INHIBITOR II | SCRAPIE- | ELAFIN-LIKE | hypothetical | | |
| | PRECURSOR (HUSI-II) (SERINE | RESPONSIVE | PROTEIN I | Ovomucoid/PCl-1 like | | |
| | PROTEASE INHIBITOR | PROTEIN 1 | homolog [Mus | inhibitors structure | | |
| RNA | KAZAL-TYPE 2) [Homo sapiens] | PRECURSOR | musculus] | containing protein | unknown EST | NO DATA |
| Hypothalamus | | ◯ | | | | |
| Olfactory bulb | | ◯ | | | | |
| C. qradrigemi | | ◯ | | | | |
| pit1 | | ◯ | | | | |
| Cortex | | ◯ | | | | |
| Diencepharose | ◯ | ◯ | | | | |
| Corpus striatum | | ◯ | | | | |
| Hippocampus | ◯ | ◯ | | | | |
| Cerebellum | | | | | | |
| m. oblongatu | | ◯ | | | | |
| aorate and vein | | | | | | |
| thymus | | | ◯ | | | |
| cecum | | | | | | |
| epididymis | ◯ | | | | | |
| testis | | | | | | |
| tongue | ◯ | | | | | |
| kidney | | | ◯ | 2 bands | | |
| skin | ◯ | ◯ | | 2 bands | | 3 bands |
| lung | ◯ | ◯ | | | ◯ | |
| heart | ◯ | ◯ | | | ◯ | |
| liver | | | | ◯ | ◯ | |
| stomach | | | | | | 3 bands |
| pancreas | | | ◯ | ◯ | | |
| small intestine | | | | | | |

Remarks:
Blank colums shows that the expression of the gene was not detected.
“NO DATA” shows that the information of the genes is not disclosed in FANTOM.
“Un known EST” and “unclassifiable” are the Curated Gene name disclosed in FANTOM.
In exprements, both of RT− and RT+ were performed. In most expreriments, bands were detected for RT+. However, in the experiment using Primer 1, bands were detected for RT−.

TABLE 3-3
Transription pattern for each clone in various organs

| | Primer | | | | |
|---|---|---|---|---|---|
| | 70 | 73 | 74 | 89 | 100 |
| | Clone ID | | | | |
| | 2310014H11 | A530065I17 | 1700011J22 | 9130004I05 | B230114O10 |
| | | hypothetical | | | inferred: RIKEN |
| | | Immunoglobulin structure | | | cDNA 1810014B01 |
| RNA | NO DATA | containing protein | NO DATA | unclassifiable | gene |
| Hypothalamus | | ◯ | | | ◯ |
| Olfactory bulb | | ◯ | | | ◯ |
| C. qradrigemi | | ◯ | ◯ | | ◯ |
| pit1 | | ◯ | | | ◯ |
| Cortex | | ◯ | | | ◯ |
| Diencepharose | | ◯ | | | ◯ |
| Corpus striatum | | ◯ | ◯ | | ◯ |
| Hippocampus | | | | | ◯ |
| Cerebellum | | | | | |
| m. oblongatu | | ◯ | | | ◯ |
| aorate and vein | | | | | |
| thymus | | | | | |
| cecum | | ◯ | | ◯ | |
| epididymis | | | | | |
| testis | | | ◯ | | |
| tongue | ◯ | | | | |
| kidney | | | | ◯ | |
| skin | ◯ | ◯ | | | ◯ |
| lung | | ◯ | | | ◯ |
| heart | | | | | ◯ |
| liver | | | | | ◯ |
| stomach | ◯ | ◯ | | ◯ | ◯ |
| pancreas | | | | | ◯ |
| small intestine | | | | ◯ | ◯ |

Remarks:
Blank colums shows that the expression of the gene was not detected.
“NO DATA” shows that the information of the genes is not disclosed in FANTOM.
“Un known EST” and “unclassifiable” are the Curated Gene name disclosed in FANTOM.
In exprements, both of RT− and RT+ were performed. In most expreriments, bands were detected for RT+. However, in the experiment using Primer 1, bands were detected for RT−.

### Example 2

2) In Vitro Transcription/Translation

<Reagents and Instruments>

Redivue [³⁵S] Methionine (>1000 Ci/mmol) at 10 mCi/ml (Amersham Pharmacia)

TNT Coupled Reticulocyte Lysate System (Promega)

Canine Pancreatic Microsomal Membranes (Promega)

RNasin Ribonuclease Inhibitor (Promega)

Methanol (special grade reagent) (Wako Pure Chemical Industries)

Acetic acid (special grade reagent) (Wako Pure Chemical Industries)

Glycerol (special grade reagent) (Wako Pure Chemical Industries)

Quick CBB (Wako Pure Chemical Industries)

Gel plate for electrophoresis PAG mini “Daiichi,” 10/20 (Daiichi Pure Chemicals)

OneSTEP Marker 4 (fX174/Hae III digest) (Nippon Gene)

Loading Quick λ/EcoR I+Hind III (TOYOBO)

T7 RNA Synthesis Set (Wako Pure Chemical Industries)

MODEL 583 GEL DRYER (BIO-RAD)

VA-810 FREEZE TRAP (TAITEC)

OIL ROTARY VACUUM PUMP GCD-135 XA (ULVAC)

PIC-200 Peltier Thermal Cycler (MJ RESEARCH)

POWER PAC 300 (BIO-RAD)

MODEL BE-222 (BIO CRAFI)

BAS-2500 (FUJIFILM)

Mupid-21 (Mini-Gel Electrophoresis System) (COSMO BIO)

Agarose-1 (Wako Pure Chemical Industries)

Mark12 MW Standard (Invitrogen)

<Method>

The expression of target clones and the presence of signal sequences was confirmed in cell-free protein expression systems.

Even though target proteins are expressed, they have a size of approximately 6 to 10 kD and thus it is rather difficult to confirm the expression thereof by SDS-PAGE. Further, it is also difficult to label target proteins by incorporating RI-labeled amino acids due to its small molecular weight. For this reason, we have increased the molecular weight through the fusion of myoglobin proteins to the C-terminus of the target protein in order to easily observe the experiment results. It is considered that this operation does not affect N-terminal side of the target protein.

Primers were designed by the following method, and forward and reverse primers were designed as target clone-specific primers. A forward primer was designed by adding 20 nucleotides from ATG (methionine) of the ORF of the target clone to the T7Kozak of the consensus sequence “GCCAATTGCCGCCACC” so as to have a Tm of 60° C. On the other hand, a reverse primer was designed by adding approximately 20 nucleotides forward from the Term of the ORF of the clone to the consensus sequence “CATCCTTCGGTGGCGGC” for the fusion to myoglobin so as to have a Tm of 60° C. (Term was not included in the sequence). In addition, a forward primer for myoglobin gene was designed by adding 20 nucleotides from the ORF (ATG) of the myoglobin gene to the sequence (GCCGCCACCGAAGGAATG) having a complement to the sequence (CATCCTTCGGTGGCGGC) attached to the 3′ terminus of the target clone so as to have a Tm of 60° C. As a reverse primer, P8 (AGCGGATAACAATTTCACACAGGAAAC) in the vector was used. Each primer was adjusted to have a concentration of 10 μM. These designed primers are listed in Table 4.

TABLE 4
Primers used for Example 2
Fused with Myoglobin

| No | Clone ID | Name | Sequence |
|---|---|---|---|
| 4 | C230071E12 | MyoR4 | CATTCCTTCGGTGGCGGCGTGGCCGGCGTGAACAGG |
| 6 | 9230111O07 | MyoR6 | CATTCCTTCGGTGGCGGCTTTTCTTTCAAAAAATTTCCCATTT |
| 9 | 1700056N09 | MyoR9 | CATTCCTTCGGTGGCGGCGAGTCCCTCATTTGTGACAC |
| 13 | A430045L05 | MyoR13 | CATTCCTTCGGTGGCGGCCACAGGATGGACAATGTTCC |
| 32 | A030004E11 | MyoR32-1 | CATTCCTTCGGTGGCGGCCTAGGCAGCTCGAAGCAGTG |
| | | MyoR32-2 | CATTCCTTCGGTGGCGGCGCTAGGTACTCCCAGGACTC |
| 45 | B230352020 | MyoR45 | CATTCCTTCGGTGGCGGCGTGGTTGTTGCAGGGGATC |
| 52 | C630041L24 | MyoR52 | CATTCCTTCGGTGGCGGCGTAACTTTTATCAGTGTTCAG |
| 64 | 2310031C01 | MyoR64 | CATTCCTTCGGTGGCGGCCTTGGCTCCTGCTTTGGAG |
| 69 | 1110005I17 | MyoR69 | CATTCCTTCGGTGGCGGCGGTCTTAGGGAAGAAGTCCC |
| 70 | 2310014H11 | MyoR70 | CATTCCTTCGGTGGCGGCGGAGGTCACTTTTGAAGGTTG |
| 73 | A530065I17 | MyoR73 | CATTCCTTCGGTGGCGGCCCCTGCCAGGCACTCGAG |
| 74 | 1700011J22 | MyoR74 | CATTCCTTCGGTGGCGGCGAACATCCACGGCTTTGGC |
| 89 | 9130004105 | MyoR89 | CATTCCTTCGGTGGCGGCCTGGGCAGTAGATATATCAAAG |
| 100 | B230114O10 | MyoR100 | CATTCCTTCGGTGGCGGCCTGCTGTGAAACATGAGGTTC |
| Myoglobin | 9830004A21 | MyoF | GCCGCCACCGAAGGAATGGGGCTCAGTGATGGGG |
| No | Clone ID | Name | Seacence |
| 4 | C230071E12 | InF 4 | GCCAATTGCCGCCACCATGTGGCACAACGTGGGGC |
| 6 | 9230111O07 | InF 6 | GCCAATTGCCGCCACCATGAAGCCCTCGTGGTTCC |
| 9 | 1700056N09 | InF 9 | GCCAATTGCCGCCACCATGTTCGACTTGAGGACGAAG |
| 13 | A430045L05 | InF 13 | GCCAATTGCCGCCACCATGGAGAAGCTGTTTGTCTTG |
| 32 | A030004E11 | In 32-1 | GCCAATTGCCGCCACCATGATGAGGCCCCTCCTGG |
| | | In 32-2 | GCCAATTGCCGCCACCATGGGCCAGCGCCTCTATC |
| 45 | B230352O20 | InF 45 | GCCAATTGCCGCCACCATGATGAAATCTGTGGTACTTG |
| 52 | C630041L24 | InF 52 | GCCAATTGCCGCCACCATGAAGGTGATCTTCTCAGTTG |
| 64 | 2310031C01 | InF 64 | GCCAATTGCCGCCACCATGGGCGCCGTCTGGTCAG |
| 69 | 1110005I17 | InF 69 | GCCAATTGCCGCCACCATGAGGATCCCAATTCTTCCC |
| 70 | 2310014H11 | InF 70 | GCCAATTGCCGCCACCATGGCTGTCTCAGTTCTTCG |
| 73 | A530065I17 | InF 73 | GCCAATTGCCGCCACCATGCAGCTGGCAAGAGGAAC |
| 74 | 1700011J22 | InF 74 | GCCAATTGCCGCCACCATGAAGCTCCTGCTGCTGAC |
| 89 | 9130004I05 | InF 89 | GCCAATTGCCGCCACCATGGCCTATAAATTGCTTCAAG |
| 100 | B230114O10 | InF 100 | GCCAATTGCCGCCACCATGCCCGGGGGCGTACC |

| | |
|---|---|
| T7 Kozak | GAGCGCGCGTAATGCGAGTCACTATAGGGCCAATTGCCGCCACCATG |
| T7 Adaptor | GAGCGCGCGTAATGCGAGTCACTATAGGGC |

F:Forward
R:Reverse

(1) PCR of the Protein-Coding Region of the Myoglobin Gene

The clone ID of the plasmids used herein is “9830004A21.”

10 μl of 10×PCR Buffer, 20 μl of 5× Q-solution, 10 μl of dNTP Mix (2 mM), 0.5 μl HotStarTaq DNA Polymerase, 1 μl of template DNA (plasmid), forward primer (final concentration 100 nM), and reverse primer (final concentration 100 nM) were added to water, thereby providing 100 μl of a resultant mixture for PCR. The reaction conditions for PCR were: heating at 95° C. for 15 minutes; 30 cycles of 1 minute at 94° C., 1 minute at 55° C., and then 1 minute at 72° C.; and thereafter heating at 72° C. for 10 minutes.

(2) PCR for Fusing the Myoglobin Gene to the Target Clone

PCR was performed through three steps.

<1st PCR>

10 μl of 10×PCR Buffer, 20 μl of 5× Q-solution, 10 μl of dNTP Mix (2 mM), 0.5 μl of HotStarTaq DNA Polymerase, x μl of distilled water (final volume 100 μl), 1 μl of template DNAs (plasmids of each clone), forward primers (10 μM, final concentration 100 nM), and reverse primers (10 μM, final concentration 100 nM) were used for PCR. The reaction conditions for PCR were: heating at 95° C. for 15 minutes; 10 cycles of 1 minute at 94° C., 1 minute at 55° C., and 1 minute at 72° C.; and thereafter heating at 72° C. for 10 minutes.

<2nd PCR>(Extension PCR, Addition of T7 Promoter)

10 μl of 10×PCR Buffer, 20 μl of 5× Q-solution, 10 μl of dNTP Mix (2 mM), 0.5 μl of HotStarTaq DNA polymerase, x μl of distilled water (final volume 100 μl), 1 μl of template DNAs (1st PCR products of each clone), forward primers (10 μM, final concentration 100 nM), and reverse primers (10 μM, final concentration 100 nM) were used for PCR. The reaction conditions for PCR were: heating at 95° C. for 15 minutes; 10 cycles of 1 minute at 94° C., 1 minute at 65° C., and 1 minute at 72° C.; 5 cycles of 1 minute at 94° C., 1 minute 63° C., and 1 minute at 72° C.; 5 cycles of 1 minute at 94° C., 1 minute at 60° C., and 1 minute at 72° C.; 5 cycles of 1 minute at 94° C., 1 minute at 58° C., and 1 minute at 72° C.; 5 cycles of 1 minute at 94° C., 1 minute at 56° C., and 1 minute at 72° C.; and thereafter heating at 72° C. for 10 minutes.

The resultant products were subjected to 2.0% agarose gel electrophoresis, stained with EtBr, and photographed under UV-irradiation.

<3rd PCR>(Overlapping PCR)

10 μl of 10×PCR Buffer, 20 μl of 5× Q-solution, 10 μl of dNTP Mix (2 mM), 0.5 μl of HotStarTaq DNA Polymerase, x μl of distilled water (final volume 100 μl), 1 μl of template DNA (2nd PCR products of each clone), 1 μl of PCR products of myoglobin gene, forward primers (10 μM, final concentration 100 nM), and reverse primers (10 μM, final concentration 100 nM) were put into a PCR tube to perform PCR. The reaction conditions for PCR were: heating at 95° C. for 15 minutes; 10 cycles of 1 minute at 94° C., 1 minute at 65° C., and 1 minute at 72° C.; 5 cycles of 1 minute at 94° C., 1 minute at 63° C., and 1 minute at 72° C.; 5 cycles of 1 minute at 94° C., 1 minute at 60° C., and 1 minute at 72° C.; 5 cycles of 1 minute at 94° C., 1 minute at 58° C., and 1 minute at 72° C.; 5 cycles of 1 minute at 94° C., 1 minute at 56° C., and 1 minute at 72° C.; and thereafter heating at 72° C. for 10 minutes.

The resultant products were subjected to 2.0% agarose gel electrophoresis, stained with EtBr, and photographed under UV-irradiation.

(3) In Vitro Transcription/Translation

The expression of target clones and the presence of signal peptide were confirmed. Experiment kits used herein were as follows:

1) TNT Cupled Reticulocyte Lysate System

2) Canine Pancreatic Microsomal Membranes

The following two reaction systems were employed:

i) Reaction system wherein Canine Pancreatic Microsomal Membranes were introduced (Microsomal Membranes +); and

ii) Reaction system wherein Canine Pancreatic Microsomal Membranes were not introduced (Microsomal Membranes −).

i) Reaction System Wherein Canine Pancreatic Microsomal Membranes were Introduced

12.5 μl of TNT Lysate, 0.5 μl of TNT Reaction Buffer, and 0.5 μl of Amino Acid Mixture/Minus Methionine, 0.5 μl of RNasin Ribonuclease Inhibitor, 0.5 μl of TNT RNA Polymerase, 2 μl of [³⁵S] Methionine, 8 μl of PCR products having proteins fused to each clone, and 2 μl of Canine Pancreatic Microsomal Membranes were put into a 0.5 ml Eppendorf tube and incubated at 30° C. for 90 minutes.

ii) Reaction System Wherein Canine Pancreatic Microsomal Membranes were not Introduced

12.5 μl of TNT lysate, 1.0 μl of TNT Reaction Buffer, 0.5 μl of Amino Acid Mixture/Minus Methionine, 0.5 μl of RNasin ribonuclease Inhibitor, 0.5 μl of TNT RNA polymerase, 1 μl of [³⁵S] Methionine, 8 μl of PCR products having proteins fused with each clone, and 9 μl of water were put into a 0.5 ml Eppendorf tube, and incubated at 30° C. for 90 minutes.

The addition of canine pancreatic microsomal membranes to the reaction system is likely to reduce the expression level. Therefore, when SDS-PAGE was performed, a smaller amount of the reaction solution of the reaction system having no microsomal membranes was applied. In contrast, a larger amount of the reaction solution of the reaction system having microsomal membranes was applied. The ratio between the reaction system wherein microsomal membranes were not introduced and the reaction system wherein microsomal membranes were introduced was 1:4. Further, samples to be applied to the gel were as follows.

1) Microsomal Membranes −

2) Microsomal Membranes +

3) Microsomal Membranes −, + (mixed)

Each sample, sample buffer, and water (final volume 10 μl) were put into a 0.5 ml Eppendorf tube and heat-denatured at 100° C. for 3 minutes. The denatured sample was subjected to SDS-PAGE with 10/20 gradient gel. After the electrophoresis, the gel was fixed and dried, and then exposed to BAS-2500 film for 30 minutes or more for imaging.

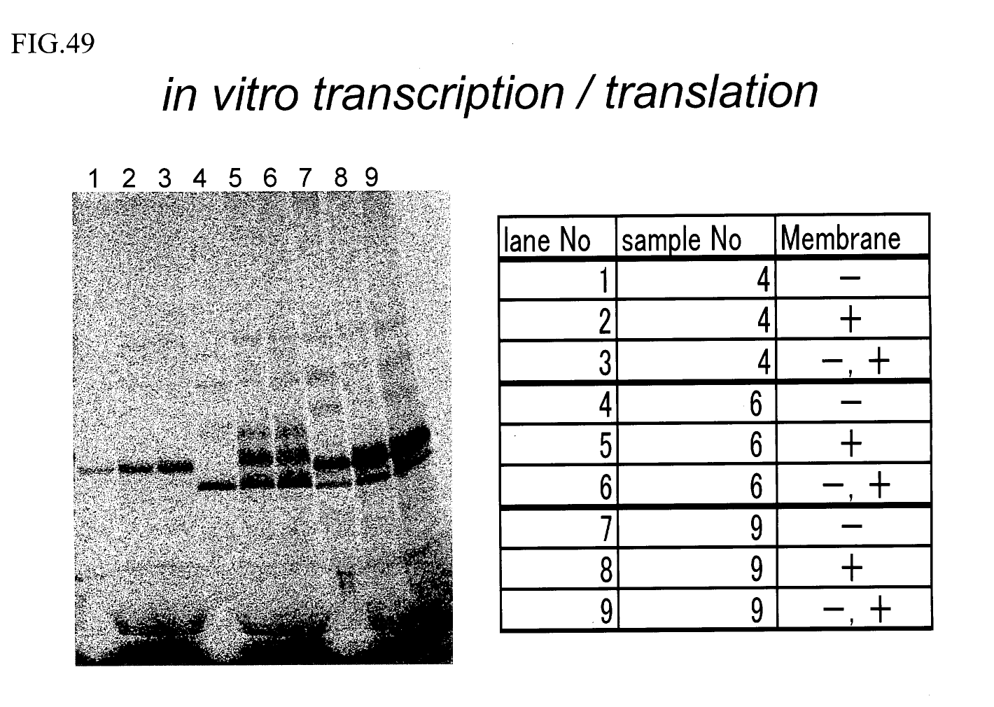

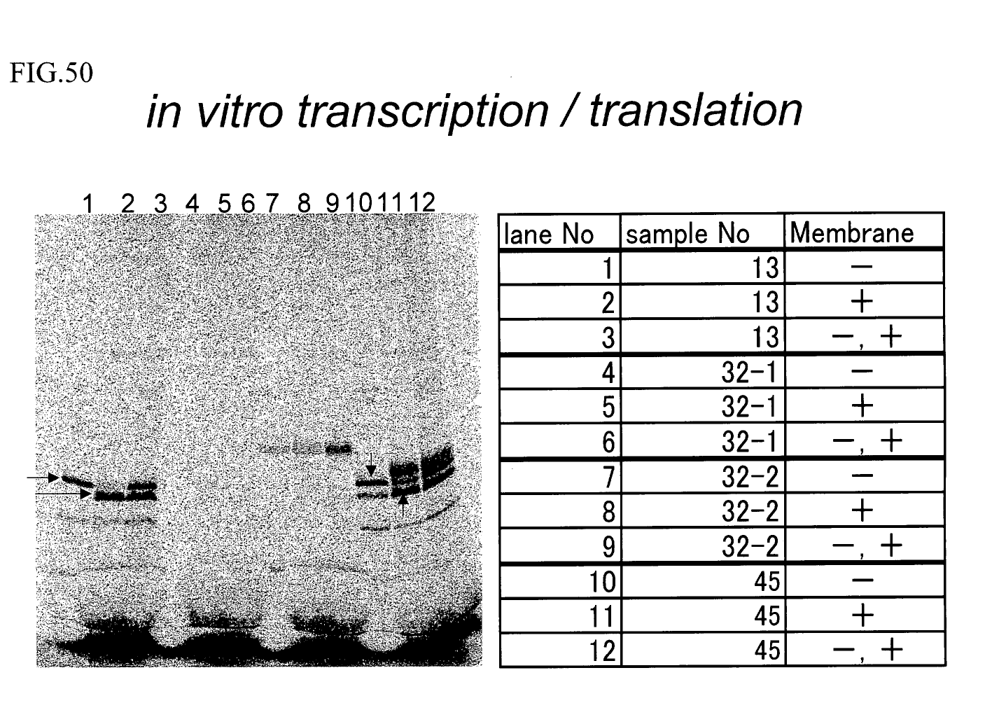

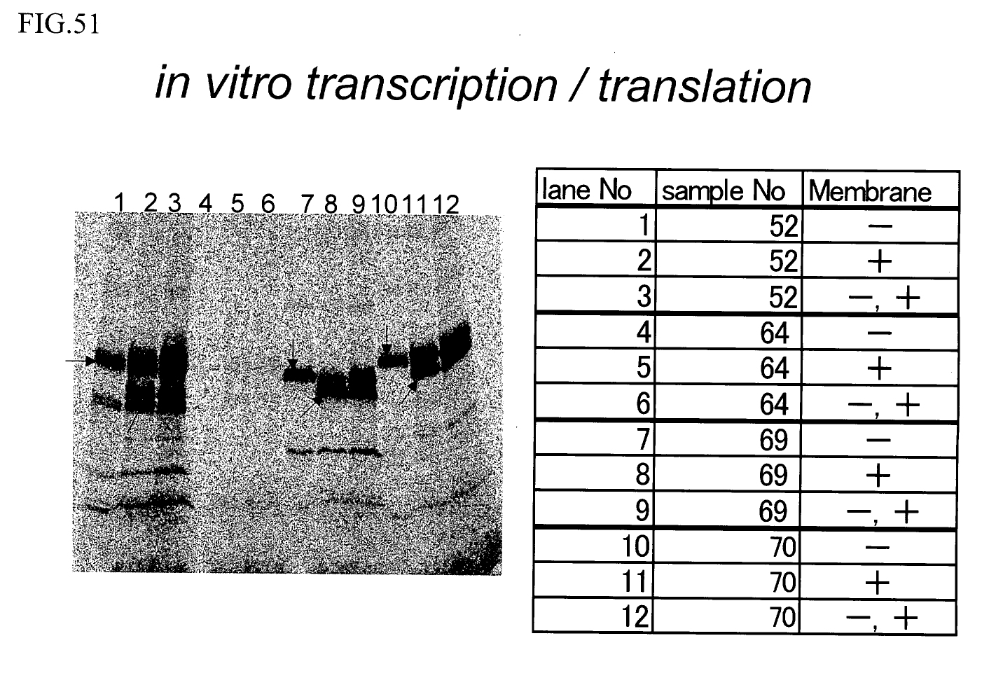

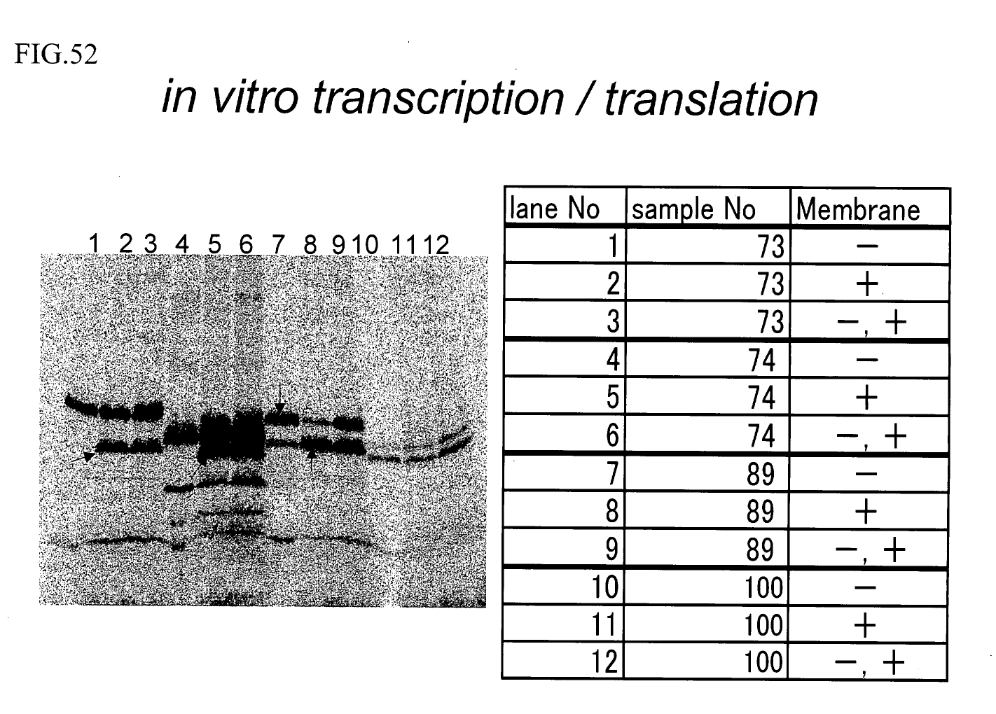

The clones used for Example 2 of which ID Nos. are C230071E12, 9230111O07, 1700056N09, A430045L05, A030004E11, B230352O020, C630041L24, 2310031C01, 1110005I17, 2310014H11, A530065I17, 1700011J22, 9130004I05 and B230114O10 include peptide signal like sequence. FIGS. 49 to 52 demonstrate the in vitro transcription/translation pattern of each clone. The figures shows the SDS-PAGE pattern of the transcribed products. For each clone, the in vitro transcription/translation pattern with and without microsomal membrane is shown. The signal peptide is cleaved by the signal peptidase activity of the microsomal membrane. If the molecular weight of the product with microsomal membrane is smaller than that without Microbial membrane, the larger product includes a signal peptide. The product with a signal peptide and the product without the signal peptide is indicated by an arrow. As these figures indicate, Sample Nos. 13, 45, 52, 69, 70, 73, 74 and 89 of which clone ID Nos. are A430045L05, B230352O020, C630041L24, 1110005I17, 2310014H11, A530065I17 and 1700011J22, respectively, were clearly cleaved by the microsomal memebrane.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### INDUSTRIAL APPLICABILITY

The present invention provides polynucleotides encoding short polypeptides which have various functions. The polynucleotides of the present inventions include a nucleotide encoding signal peptides. These clones would be expressed and secreted outside a cell and function for in a living body.

Among these polynucleotides, some polynucleotides are expressed in various tissues or organs as the example demonstrates. It is suggested that these polynucleotides are relating to the tissues or organs in which the polynucleotides are expressed.

As Table 3 demonstrates, the functions of the clone ID Nos A030004E11, 1700007F22, B230352O20, D630020P16, C630041L24, A530065I17 shows the applicability of the clones.

What is claimed is:

1. The isolated polynucleotide having a nucleotide sequence of a clone selected from the group consisting of 1110005I17 (SEQ ID NO: 1), 1700007F22 (SEQ ID NO: 2), 1700011J22 (SEQ ID NO: 3), 1700056N09 (SEQ ID NO: 4), 2310014H11 (SEQ ID NO: 5), 2310031C01 (SEQ ID NO: 6), 4930563B01 (SEQ ID NO: 7), 9130004I05 (SEQ ID NO: 8), 9230110A19 (SEQ ID NO: 9), 9230111O07 (SEQ ID NO: 10), A030004E11 (SEQ ID NO: 11), A430045L05 (SEQ ID NO: 12), A530065I17 (SEQ ID NO: 13), A830010B16 (SEQ ID NO:14), B230114O10 (SEQ ID NO: 15), B230352O20 (SEQ ID NO: 16), C230071E12 (SEQ ID NO: 17), C630041L24 (SEQ ID NO: 18) and D630020P16 (SEQ ID NO: 19).

2. The isolated polynucleotide according to claim 1 having a nucleotide sequence of a clone selected from the group consisting of 1700007F22 (SEQ ID NO: 2), A030004E11 (SEQ ID NO: 11), A530065I17 (SEQ ID NO: 13), B230352O20 (SEQ ID NO: 16), C630041L24 (SEQ ID NO: 18) and D630020P16 (SEQ ID NO: 19).

3. The isolated polypeptide having an amino acid sequence selected from the group consisting of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37 and SEQ ID NO:38.

4. The isolated polypeptide according to claim 4 having an amino acid sequence selected from the group consisting of SEQ ID NO:21, SEQ ID NO:30, SEQ ID NO:32, SEQ ID NO:35, SEQ ID NO:36 and SEQ ID NO:38.
